SEQUENCE LISTING (109 sequences)

SEQ ID NO: 1 - DNA, 813 bp, Homo sapiens
  source 1..813: mol_type = genomic DNA
atggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 2 - protein, 271 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDESISYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - DNA, 930 bp, Homo sapiens
  source 1..930: mol_type = genomic DNA
atgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 4 - protein, 310 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFLGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 5 - DNA, 801 bp, synthetic construct
  source 1..801: mol_type = other DNA
atggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 6 - protein, 267 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDESISYGKLTFGQGTILTVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 7 - DNA, 918 bp, synthetic construct
  source 1..918: mol_type = other DNA
atgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactca

SEQ ID NO: 8 - protein, 306 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFLGWNEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 9 - DNA, 1800 bp, synthetic construct
  source 1..1800: mol_type = other DNA
atgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactcaagagccaaaagaagcgggagcggtgcgacaaactttagcctgttgaaacaagccggcgacgttgaagagaaccccggacctatggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 10 - protein, 600 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFLGWNEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDESISYGKLTFGQGTILTVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 11 - DNA, 810 bp, synthetic construct
  source 1..810: mol_type = other DNA
atggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 12 - protein, 270 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDESISYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 13 - DNA, 930 bp, synthetic construct
  source 1..930: mol_type = other DNA
atgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttacttcaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggacttt

SEQ ID NO: 14 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFLGWNEKLFFGSGTQLSVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 15 - DNA, 1830 bp, synthetic construct
  source 1..1830: mol_type = other DNA
atgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttacttcaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 16 - protein, 610 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFLGWNEKLFFGSGTQLSVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDESISYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 17 - DNA, 828 bp, Homo sapiens
  source 1..828: mol_type = genomic DNA
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 18 - protein, 276 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - DNA, 936 bp, Homo sapiens
  source 1..936: mol_type = genomic DNA
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 20 - protein, 312 aa, Homo sapiens
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - DNA, 816 bp, synthetic construct
  source 1..816: mol_type = other DNA
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 22 - protein, 272 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 23 - DNA, 924 bp, synthetic construct
  source 1..924: mol_type = other DNA
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactca

SEQ ID NO: 24 - protein, 308 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - DNA, 1821 bp, synthetic construct
  source 1..1821: mol_type = other DNA
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatcttcgaaacgtaacccctccaaaagtgagtctctttgaaccgagtaaggctgagatcgcgaacaaacaaaaggcgaccctcgtctgtcttgcgcgaggattttttcccgaccacgtggagttgtcttggtgggtaaacggtaaggaagtacacagcggtgtttgcaccgaccctcaagcctacaaggaatctaactattcatactgcctttcatcccgacttagggtttctgctaccttttggcacaatccgaggaatcactttaggtgtcaagtacagttccacggattgtcagaggaggataaatggccggagggctccccgaagccggttacgcagaacattagtgcggaagcctggggacgagcagactgcggtatcacgtctgccagctatcagcaaggcgttctgtcagcgacaattctgtacgaaatacttttgggtaaggctacattgtatgcggtattggtgtctacgctggtagtcatggccatggtgaaacgaaaaaactcaagagccaaaagaagcgggagcggtgcgacaaactttagcctgttgaaacaagccggcgacgttgaagagaaccccggacctatggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacattcaaaacccagaacccgccgtctaccagctgaaagacccgaggtctcaagactctacgttgtgcttgttcaccgatttcgacagtcagataaatgtgcctaagaccatggagagtggcactttcatcactgacaaatgtgtgttggacatgaaggctatggacagcaagtcaaacggcgcgattgcttggtccaaccaaacttctttcacgtgccaggacatcttcaaggagacaaacgccacctatccatcctctgatgttccgtgcgatgcgactcttaccgagaaaagcttcgagacggacatgaacttgaacttccaaaacctgcttgtgatggtactgcgaatacttcttcttaaggtggcgggcttcaatttgctcatgacactcagactttggtctagc

SEQ ID NO: 26 - protein, 607 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVMVLRILLLKVAGFNLLMTLRLWSS

SEQ ID NO: 27 - DNA, 828 bp, synthetic construct
  source 1..828: mol_type = other DNA
atggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 28 - protein, 276 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 29 - DNA, 936 bp, synthetic construct
  source 1..936: mol_type = other DNA
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggacttt

SEQ ID NO: 30 - protein, 312 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 31 - DNA, 1854 bp, synthetic construct
  source 1..1854: mol_type = other DNA
atgagcccaattttcacctgcatcacaatcctttgtctgctggctgcaggttctcctggtgaagaagtcgcccagactccaaaacatcttgtcagaggggaaggacagaaagcaaaactttattgtgccccaattaaaggacacagttatgttttctggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctactaagcttgaggattcagcagtgtatttttgtgccagcagccaatcacggagccttaggggcactgaagctttctttggacaaggcaccagactcacagttgttgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaagaagcgggagcggtgcgacaaactttagcctgttgaaacaagccggcgacgttgaagagaaccccggacctatggagaagaatcctttggcagccccacttcttatcctctggtttcatcttgactgcgtgagcagcattctgaacgtggaacaaagtcctcagtcactgcatgttcaggagggagacagcaccaatttcacctgcagcttcccttccagcaatttttatgcccttcactggtacagatgggaaactgcaaaaagccccgaggccttgtttgttatgactcttaatggggatgaaaagaagaaaggacgcattagtgccactcttaataccaaggagggttacagctatttgtatatcaaaggatcccagcctgaggactcagccacatacctctgtgcctccggaagtggtggtgctacaaacaagctcatctttggaactggcactctgcttgctgtccagccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 32 - protein, 618 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQSRSLRGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASGSGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 33 - DNA, 810 bp, synthetic construct
  source 1..810: mol_type = other DNA
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccacagatgtgggcacctacttctgcgctggcatcggtagcagcaacaccggtaagctcatctttgggcaagggacaactttacaagtaaaaccagacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 34 - protein, 270 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGIGSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 35 - DNA, 960 bp, synthetic construct
  source 1..960: mol_type = other DNA
atggatacctggctcgtgtgttgggccatctttagcctgctgaaggccggactgaccgagcctgaagtgacccagactccaagccatcaagtgactcagatggggcaagaagtcattctgcgttgcgtgcccatcagcaaccacctgtacttttattggtatcgccagatcctgggccagaaagtggaattcctggtgtccttctacaacaatgagatctccgagaagtccgagatcttcgacgaccagttctccgtggaaagacccgacggcagcaacttcacactgaagatccggtctaccaaacttgaggactccgctatgtatttttgtgcaatcacaggtcgcgtttcatatgagcaatatttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 36 - protein, 320 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCAITGRVSYEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 37 - DNA, 1823 bp, synthetic construct
  source 1..1823: mol_type = other DNA
atacctggctcgtgtgttgggccatctttagcctgctgaaggccggactgaccgagcctgaagtgacccagactccaagccatcaagtgactcagatggggcaagaagtcattctgcgttgcgtgcccatcagcaaccacctgtacttttattggtatcgccagatcctgggccagaaagtggaattcctggtgtccttctacaacaatgagatctccgagaagtccgagatcttcgacgaccagttctccgtggaaagacccgacggcagcaacttcacactgaagatccggtctaccaaacttgaggactccgctatgtatttttgtgcaatcacaggtcgcgtttcatatgagcaatatttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccacagatgtgggcacctacttctgcgctggcatcggtagcagcaacaccggtaagctcatctttgggcaagggacaactttacaagtaaaaccagacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 38 - protein, 609 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCAITGRVSYEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGIGSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 39 - DNA, 930 bp, Homo sapiens
  source 1..930: mol_type = genomic DNA
atgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctga

SEQ ID NO: 40 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 41 - DNA, 1101 bp, Homo sapiens
  source 1..1101: mol_type = genomic DNA
atgcgcgtcatggctccacgcgccctcctcctgctgctctcagggggcctggccctgaccgagacctgggcctgctcccactccatgcgctatttcgacaccgccgtgtcccgcccaggccgcggtgagccacgcttcatctcagtgggctacgtggacgacactcagttcgtgcgcttcgacagcgacgccgccagtcctcgcggggagccacgcgcaccatgggtggagcaggaggggcctgagtattgggaccgcgagacacagaagtacaagcgccaggcacaggctgaccgcgtgagcctgcgcaacctgcgcggctactacaaccagagcgaggacgggtctcacaccctccagcgcatgtctggctgcgacctggggcctgacgggcgcctcctccgcgggtatgaccagtccgcctacgacggcaaggattacatcgccctgaacgaggacctgcgctcctggaccgccgctgacaccgccgctcagatcacccagcgcaagttggaggctgcccgcgccgctgagcagctgcgcgcctacctggagggcacatgcgtggagtggctccgccgctacctggagaacgggaaggagaccctgcagcgcgcagaaccaccaaagacacacgtgacccaccaccctctctctgaccatgaggccaccctgcgctgctgggccctgggcttctaccctgctgagatcacactgacctggcagcgcgatggggaggaccagacccaggacaccgagcttgtggagacccgcccagcaggtgatggcaccttccagaagtgggcagctgtggtggtgccttctgggcaagagcagcgctacacatgccatatgcagcacgaggggctgcaagagccactcaccctgagctgggagccatcttcccagccaaccatcccaatcatgggcatcgttgctggcctggctgtcctggttgtcctggctgtccttggtgctgtggtcaccgctatgatgtgtcgccgcaagagctcaggtgggaaaggtgggagctgctctcaggctgcatgcagcaacagtgcccagggctctgatgagtctctcatcacttgtaaagcctga

SEQ ID NO: 42 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 43 - DNA, 9683 bp, synthetic construct
  source 1..9683: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggcaccagcctcctctgctggatggccctgtgtctcctgggggcagatcacgcagatactggagtctcccaggaccccagacacaagatcacaaagaggggacagaatgttactttcaggtgtgatccaatttctgaacacaaccgcctttattggtaccgccagaccctggggcagggcccagagtttctgacttacttccagaatgaagctcaacttgaaaaatcaaggctgctcagtgatcggttctctgcagagaggcctaagggatctttctccaccttggagatccagcgcacagagcagggggactctgccatgtatctctgtgccagcagccgcactgctggagatactcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctcttgggcctgcttatcctttggctgcagctgcaatgggtgagcagcaaacaggaggtgactcagattcctgcagctctgagtgtcccagaaggagaaaacttggttctcaactgcagtttcactgatagcgctatttacaacctccagtggtttaggcaggaccctgggaaaggcctcacatctctgttgcttattcagtcaagtcagagagagcaaacaagtggacgccttaatgcctctctggataaatcatcaggacgcagtactctttacattgcagcttctcagcctggtgattcagccacctacctgtgcgctgtgaggggtggtacctcaggaacctacaaatacatctttggaacaggcaccaggctgaaggttcttgcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 44 - DNA, 1122 bp, Homo sapiens
  source 1..1122: mol_type = genomic DNA
atgcctcccgttccaggcgttccattccgcaacgttgacaacgactccccgacctcagttgagttagaagactgggtagatgcacagcatcccacagatgaggaagaggaggaagcctcctccgcctcttccactttgtacttagtattttccccctcttctttctccacatcctcttctctgattcttggtggtcctgaggaggaggaggtgccctctggtgtgataccaaatcttaccgagagcattcccagtagtcctccacagggtcctccacagggtccttcccagagtcctctgagctcctgctgctcctctttttcatggagctcattcagtgaggagtccagcagccagaaaggggaggatacaggcacctgtcagggcctgccagacagtgagtcctctttcacatatacactagatgaaaaggtggccgagttagtggagttcctgctcctcaaatacgaagcagaggagcctgtaacagaggcagagatgctgatgattgtcatcaagtacaaagattactttcctgtgatactcaagagagcccgtgagttcatggagcttctttttggccttgccctgatagaagtgggccctgaccacttctgtgtgtttgcaaacacagtaggcctcaccgatgagggtagtgatgatgagggcatgcccgagaacagcctcctgattattattctgagtgtgatcttcataaagggcaactgtgcctctgaggaggtcatctgggaagtgctgaatgcagtaggggtatatgctgggagggagcacttcgtctatggggagcctagggagctcctcactaaagtttgggtgcagggacattacctggagtatcgggaggtgccccacagttctcctccatattatgaattcctgtggggtccgagagcccattcagaaagcatcaagaagaaagtactagagtttttagccaagctgaacaacactgttcctagttcctttccatcctggtacaaggatgctttgaaagatgtggaagagagagtccaggccacaattgataccgcagatgatgccactgtcatggccagtgaaagcctcagtgtcatgtccagcaacgtctccttttctgagtga

SEQ ID NO: 45 - protein, 373 aa, Homo sapiens
MPPVPGVPFRNVDNDSPTSVELEDWVDAQHPTDEEEEEASSASSTLYLVFSPSSFSTSSSLILGGPEEEEVPSGVIPNLTESIPSSPPQGPPQGPSQSPLSSCCSSFSWSSFSEESSSQKGEDTGTCQGLPDSESSFTYTLDEKVAELVEFLLLKYEAEEPVTEAEMLMIVIKYKDYFPVILKRAREFMELLFGLALIEVGPDHFCVFANTVGLTDEGSDDEGMPENSLLIIILSVIFIKGNCASEEVIWEVLNAVGVYAGREHFVYGEPRELLTKVWVQGHYLEYREVPHSSPPYYEFLWGPRAHSESIKKKVLEFLAKLNNTVPSSFPSWYKDALKDVEERVQATIDTADDATVMASESLSVMSSNVSFSE

SEQ ID NO: 46 - DNA, 1089 bp, Homo sapiens
  source 1..1089: mol_type = genomic DNA
atgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttga

SEQ ID NO: 47 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 48 - protein, 98 aa, Alphapapillomavirus sp.
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 49 - DNA, 1098 bp, Homo sapiens
  source 1..1098: mol_type = genomic DNA
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 50 - protein, 365 aa, Homo sapiens [sequence omitted]

SEQ ID NO: 51 - DNA, 8786 bp, synthetic construct
  source 1..8786: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatggatacctggctcgtgtgttgggccatctttagcctgctgaaggccggactgaccgagcctgaagtgacccagactccaagccatcaagtgactcagatggggcaagaagtcattctgcgttgcgtgcccatcagcaaccacctgtacttttattggtatcgccagatcctgggccagaaagtggaattcctggtgtccttctacaacaatgagatctccgagaagtccgagatcttcgacgaccagttctccgtggaaagacccgacggcagcaacttcacactgaagatccggtctaccaaacttgaggactccgctatgtatttttgtgcaatcacaggtcgcgtttcatatgagcaatatttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccacagatgtgggcacctacttctgcgctggcatcggtagcagcaacaccggtaagctcatctttgggcaagggacaactttacaagtaaaaccagacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagtagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 52 - protein, 8 aa, synthetic construct
VRFFFPSL

SEQ ID NO: 53 - protein, 9 aa, synthetic construct
FFFPSLREA

SEQ ID NO: 54 - protein, 10 aa, synthetic construct
ARVRFFFPSL

SEQ ID NO: 55 - protein, 9 aa, synthetic construct
VRFFFPSLR

SEQ ID NO: 56 - protein, 9 aa, synthetic construct
RVRFFFPSL

SEQ ID NO: 57 - protein, 8 aa, synthetic construct
FFPSLREA

SEQ ID NO: 58 - protein, 11 aa, synthetic construct
ARVRFFFPSLR

SEQ ID NO: 59 - protein, 8 aa, synthetic construct
SARVRFFF

SEQ ID NO: 60 - protein, 11 aa, synthetic construct
VRFFFPSLREA

SEQ ID NO: 61 - protein, 10 aa, synthetic construct
RFFFPSLREA

SEQ ID NO: 62 - protein, 8 aa, synthetic construct
RAREFMEL

SEQ ID NO: 63 - protein, 9 aa, synthetic construct
RAREFMELL

SEQ ID NO: 64 - protein, 10 aa, synthetic construct
RAREFMELLF

SEQ ID NO: 65 - protein, 10 aa, synthetic construct
LKRAREFMEL

SEQ ID NO: 66 - protein, 9 aa, synthetic construct
VILKRAREF

SEQ ID NO: 67 - protein, 9 aa, synthetic construct
FPVILKRAR

SEQ ID NO: 68 - protein, 9 aa, synthetic construct
KRAREFMEL

SEQ ID NO: 69 - protein, 10 aa, synthetic construct
KRAREFMELL

SEQ ID NO: 70 - protein, 11 aa, synthetic construct
LKRAREFMELL

SEQ ID NO: 71 - protein, 11 aa, synthetic construct
RAREFMELLFG

SEQ ID NO: 72 - protein, 9 aa, synthetic construct
YMLDLQPET

SEQ ID NO: 73 - DNA, 810 bp, Homo sapiens
  source 1..810: mol_type = genomic DNA
atggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 74 - protein, 270 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 75 - DNA, 927 bp, Homo sapiens
  source 1..927: mol_type = genomic DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 76 - protein, 309 aa, Homo sapiens
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 77 - DNA, 807 bp, synthetic construct
  source 1..807: mol_type = other DNA
atggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 78 - protein, 269 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 79 - DNA, 960 bp, synthetic construct
  source 1..960: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 80 - protein, 320 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 81 - DNA, 1824 bp, synthetic construct
  source 1..1824: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 82 - protein, 608 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 83 - protein, 9 aa, synthetic construct
KVLEYVIKV

SEQ ID NO: 84 - DNA, 825 bp, Homo sapiens
  source 1..825: mol_type = genomic DNA
atggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 85 - protein, 275 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - DNA, 966 bp, Homo sapiens
  source 1..966: mol_type = genomic DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 87 - protein, 322 aa, Homo sapiens
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 88 - DNA, 822 bp, synthetic construct
  source 1..822: mol_type = other DNA
atggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 89 - protein, 274 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 90 - DNA, 999 bp, synthetic construct
  source 1..999: mol_type = other DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 91 - protein, 333 aa, synthetic construct
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 92 - DNA, 1878 bp, synthetic construct
  source 1..1878: mol_type = other DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 93 - protein, 626 aa, synthetic construct
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 94 - DNA, 816 bp, Homo sapiens
  source 1..816: mol_type = genomic DNA
atggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 95 - protein, 272 aa, Homo sapiens
METLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - DNA, 927 bp, Homo sapiens
  source 1..927: mol_type = genomic DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 97 - protein, 309 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 98 - DNA, 813 bp, synthetic construct
  source 1..813: mol_type = other DNA
atggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 99 - protein, 271 aa, synthetic construct
METLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 100 - DNA, 960 bp, synthetic construct
  source 1..960: mol_type = other DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 101 - protein, 320 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 102 - DNA, 1830 bp, synthetic construct
  source 1..1830: mol_type = other DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 103 - protein, 610 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMETLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 104 - protein, 9 aa, synthetic construct
SLLQHLIGL

SEQ ID NO: 105 - DNA, 8055 bp, synthetic construct
  source 1..8055: mol_type = other DNA
gaattcgtcgacgctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccaattacattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaataggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatggatacctggctcgtgtgttgggccatctttagcctgctgaaggccggactgaccgagcctgaagtgacccagactccaagccatcaagtgactcagatggggcaagaagtcattctgcgttgcgtgcccatcagcaaccacctgtacttttattggtatcgccagatcctgggccagaaagtggaattcctggtgtccttctacaacaatgagatctccgagaagtccgagatcttcgacgaccagttctccgtggaaagacccgacggcagcaacttcacactgaagatccggtctaccaaacttgaggactccgctatgtatttttgtgcaatcacaggtcgcgtttcatatgagcaatatttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccacagatgtgggcacctacttctgcgctggcatcggtagcagcaacaccggtaagctcatctttgggcaagggacaactttacaagtaaaaccagacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatggaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattactagtggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtccagtgtggtggaattctgcagatatcacggctagcgccaccatgggtcgggggctgctcaggggcctgtggccgctgcacatcgtcctgtggacgcgtatcgccagcacgatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtgaaccggcagcagaaggctagtggttcaggcgcaacgaatttctctttgctgaagcaggctggggatgtcgaagaaaatccgggtccaatggtgggctcgctcaactgcatcgtagcagtctcccagaatatgggcatcgggaagaacggtgatttcccgtggcccccacttcgcaacgagagccgttatttccaaagaatgactacaacctcctccgtggagggtaagcagaacctggtcatcatggggaagaagacctggttctctatccctgaaaaaaaccgccccctgaagggccgcatcaacctggtgctgagcagggaactcaaggagcctcctcagggcgcgcattttctgagccgctcattggatgacgctctcaaactgaccgaacagccggagctagccaacaaggtggacatggtgtggatcgtcggaggctcctccgtgtacaaggaggccatgaatcaccccggccacttgaagctgttcgtcacccggatcatgcaggacttcgagtcggacacgttctttccagagattgacctggagaagtacaagctgctgcccgagtacccgggagttcttagtgatgtgcaggaggagaaaggcatcaagtacaaatttgaggtgtacgagaagaacgactaacggtccgtcctgaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgacaagctt

SEQ ID NO: 106 - protein, 10 aa, synthetic construct
YMLDLQPETT

SEQ ID NO: 107 - protein, 8 aa, synthetic construct
VLEYVIKV

SEQ ID NO: 108 - protein, 8 aa, synthetic construct
KVLEYVIK

SEQ ID NO: 109 - DNA, 8055 bp, synthetic construct
  source 1..8055: mol_type = other DNA
gaattcgtcgacgctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccaattacattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaataggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatggatacctggctcgtgtgttgggccatctttagcctgctgaaggccggactgaccgagcctgaagtgacccagactccaagccatcaagtgactcagatggggcaagaagtcattctgcgttgcgtgcccatcagcaaccacctgtacttttattggtatcgccagatcctgggccagaaagtggaattcctggtgtccttctacaacaatgagatctccgagaagtccgagatcttcgacgaccagttctccgtggaaagacccgacggcagcaacttcacactgaagatccggtctaccaaacttgaggactccgctatgtatttttgtgcaatcacaggtcgcgtttcatatgagcaatatttcgggccgggcaccaggctcacggtcacagaagatctcaataaagtgttcccccctgaggttgcggtgtttgagccgtccaaagcggagattgcccacacacagaaagcgactttggtttgtttggcgacaggctttttccctgaccacgtagagctgtcttggtgggtcaacggcaaggaggttcacagcggtgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccacagatgtgggcacctacttctgcgctggcatcggtagcagcaacaccggtaagctcatctttgggcaagggacaactttacaagtaaaaccagacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatggaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattactagtggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtccagtgtggtggaattctgcagatatcacggctagcgccaccatgggtcgggggctgctcaggggcctgtggccgctgcacatcgtcctgtggacgcgtatcgccagcacgatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtgaaccggcagcagaaggctagtggttcaggcgcaacgaatttctctttgctgaagcaggctggggatgtcgaagaaaatccgggtccaatggtgggctcgctcaactgcatcgtagcagtctcccagaatatgggcatcgggaagaacggtgatttcccgtggcccccacttcgcaacgagagccgttatttccaaagaatgactacaacctcctccgtggagggtaagcagaacctggtcatcatggggaagaagacctggttctctatccctgaaaaaaaccgccccctgaagggccgcatcaacctggtgctgagcagggaactcaaggagcctcctcagggcgcgcattttctgagccgctcattggatgacgctctcaaactgaccgaacagccggagctagccaacaaggtggacatggtgtggatcgtcggaggctcctccgtgtacaaggaggccatgaatcaccccggccacttgaagctgttcgtcacccggatcatgcaggacttcgagtcggacacgttctttccagagattgacctggagaagtacaagctgctgcccgagtacccgggagttcttagtgatgtgcaggaggagaaaggcatcaagtacaaatttgaggtgtacgagaagaacgactaacggtccgtcctgaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgacaagctt

(19) United States
(12) Patent Application Publication
(10) Pub. No.: US 2025/0288675 A1
(43) Pub. Date: Sep. 18, 2025

(54) MULTIPLEXED T CELL RECEPTOR COMPOSITIONS, COMBINATION THERAPIES, AND USES THEREOF

(71) Applicant: TScan Therapeutics, Inc., Waltham, MA (US)

(72) Inventors: Yifan Wang, Arlington, MA (US); Cagan Gurer, Chappaqua, NY (US); Gavin MacBeath, Wakefield, MA (US)

(21) Appl. No.: 18/860,341

(22) PCT Filed: Apr. 28, 2023
(86) PCT No.: PCT/US23/20338

§ 371 Date: Oct. 25, 2024

Related U.S. Application Data
(60) Provisional application No. 63/337,522, filed on May 2, 2022.
(60) Provisional application No. 63/342,451, filed on May 16, 2022.
(60) Provisional application No. 63/413,553, filed on Oct. 5, 2022.
(60) Provisional application No. 63/423,269, filed on Nov. 7, 2022.

(51) Int. Cl. A61K 40/42; A61K 40/11; A61K 40/32; A61K 40/50; A61P 35/00; C07K 14/725; C07K 16/30

(52) U.S. Cl. CPC A61K 40/4271; A61K 40/11; A61K 40/32; A61K 40/50; A61P 35/00; C07K 14/7051; C07K 16/30; A61K 2239/29

(57) ABSTRACT

Provided herein are multiplexed T cell receptor compositions, combination therapies, and uses thereof.

### CROSS-REFERENCE TO RELATED APPLICATIONS

This application claims the benefit of priority to U.S. Provisional Application Ser. No. 63/337,522, filed on 2 May 2022; U.S. Provisional Application Ser. No. 63/342,451, filed on 16 May 2022; U.S. Provisional Application Ser. No. 63/413,553, filed on 5 Oct. 2022; and U.S. Provisional Application Ser. No. 63/423,269, filed on 7 Nov. 2022; the entire contents of each of said applications are incorporated herein in their entirety by this reference.

### SUMMARY OF THE INVENTION

The present invention is based, at least in part, on the discovery of certain binding proteins, including T cell receptors (TCRs), that in combination recognize more than one antigen (e.g., more than one antigen on the same target and/or more than one antigen on different targets), and engineered cells comprising same, can overcome antigen heterogeneity and/or human leukocyte antigen (HLA) loss-of-heterozygosity to treat cancers, including solid tumors. For example, adoptive cell transfer (ACT) with genetically engineered T cells holds great promise for treating cancers, such as solid tumors, but by targeting only one antigen at a time, complete responses have been rare and are often short-lived due to heterogeneous expression of cancer associated antigens and HLA loss-of-heterozygosity. Multiplexed TCR-T cell (TCR-T) therapy across multiple target antigens and/or HLA molecules mimics the natural oligoclonal T cell response to cancer and provides a way to address some of the major challenges associated with resistance to adoptive cell therapy. As a non-limiting, representative example, synergistic cytotoxicity was achieved using two TCRs to target mixed tumor cell cultures with heterogenous antigen expression. The presence of one TCR-T/target cell pair enhanced the activity of the other TCR-T against its target; this effect was mediated via secreted soluble factors. The results demonstrate that the use of multiplexed T cell receptors and related compositions (e.g., multiplexed TCR-T) can overcome antigen heterogeneity not only through independent targeting of different target cells in the same tumor, but also by cytokine-mediated enhancement of each T cell's response. Surprisingly, these results further demonstrate unexpectedly synergistic effect.

### BRIEF DESCRIPTION OF THE DRAWINGS

FIG. 1A-FIG. 1C show that inter- and intra-tumoral heterogeneity of target expression is a clinical challenge for T cells expressing a therapeutic TCR (TCR-T) therapy. Examples of variable inter- and intra-tumoral antigen expression in human melanoma tumor samples are provided. Immunohistochemistry was performed on human melanoma tumor microarrays using PRAME-specific antibodies (pink) and MAGEC2-specific antibodies (blue). Heterogenous antigen expression within the tumor was observed in multiple sections as represented in FIG. 1A as well as sections dominated by the presence of a single antigen (FIGS. 1B and 1C) with variable degrees of expression.

FIG. 2A and FIG. 2B show that HLA loss of heterozygosity (LOH) is common and highlights a need for multiplexed TCR-T therapy. FIG. 2A shows results of representative, non-limiting HLA LOH analysis of non-small cell lung cancer samples demonstrating the wide prevalence of clonal and partial LOH of the HLA-A*02:01 allele. FIG. 2B shows that monotherapy TCR-T frequently leads to partial responses and rapid relapse, partly due to target antigen or HLA heterogeneity. A multiplexing approach has been developed and described herein to address this issue, thereby improving long-term remission.

FIG. 3A and FIG. 3B provide a representative, non-limiting multiplexing approach to overcome target heterogeneity and show that multiplexing TCR-Ts has synergistic anti-tumor activity. FIG. 3A shows results of Incucyte® NucLightRed-labeled HPV+ (CaSki) and MAGEA1+ (A101D) cell lines were grown in the presence of HPV16 E7-TCR-T, MAGEA1-TCR-T, or a combination of both TCR-Ts. Cell growth was assessed using Incucyte® analysis over a period of three days. FIG. 3B shows results of synergistic cytotoxicity was observed between the two TCRs as calculated by % cell survival at 72 hours.

FIG. 4A-FIG. 4C further shows that multiplexing TCR-Ts has synergistic anti-tumor activity due to cytokine-mediated enhancement. FIG. 4A shows a schematic of modeling intra-tumor target expression variability using two different cell lines. FIG. 4B shows results of a co-culture of T-cells expressing a MAGEA1-specific TCR and a target cell line with high MAGEA1 expression (A2058) enhances the cytotoxicity of T cells expressing a MAGEC2 TCR against a cell line with moderate MAGEC2 expression (SKMEL5). FIG. 4C shows that increase in cytotoxicity is driven by soluble factors secreted by the T cells targeting MAGEA1, which leads to increased activation of the T cells targeting MAGEC2, as shown in the described illustrative Transwell experiment.

FIG. 5A and FIG. 5B provide a representative, non-limiting screening strategy to select patients and TCR-Ts and further illustrates and an ImmunoBank strategy enabling customized multiplexing of TCR-Ts. FIG. 5A shows an illustrative screening strategy to select patients and TCR-Ts for multiplexed TCR-T therapy. Following germline HLA genotyping, patient tumors are assessed for target expression using any of a number of well-known methods, such as immunohistochemistry (IHC) or RNA in situ hybridization (ISH). Tumor samples also may be assessed for HLA LOH by genomic sequencing. If LOH is observed, TCR-Ts are chosen that target 2 different HLAs on the intact chromosome arm. If LOH is not observed, TCR-Ts are chosen that target HLAs on opposite chromosomes. FIG. 5B shows that customized TCR-T therapies for individual cancer patients would benefit from the building of an ImmunoBank of therapeutic TCRs recognizing different targets (in rows) presented on different HLA alleles (in columns). By multiplexing across both targets and HLAs, this strategy is designed to prevent resistance arising from either target loss or HLA LOH.

FIG. 6 provides summary data of a representative example of multiplex TCR-T therapeutic addressing intratumor heterogeneity with two TCR-T therapies targeting different antigens on a single HLA. The heterogeneous expression of the cancer-associated proteins MAGE-A1 and PRAME was assessed by immunohistochemistry using antibodies specific to MAGE-A1 and PRAME. A lead TCR specific to a MAGE-A1-derived epitope presented on HLA-A*02:01 and a lead TCR specific to a PRAME-derived epitope presented on HLA-A*02:01 were used. The TCRs demonstrated high potency in vitro and in vivo and appeared highly selective for their respective peptide/MHC targets. The figure further demonstrates the value of combining the MAGE-A1-specific TCR and the PRAME-specific TCR to address a tumor model made of a mixture of MAGE-A1- or PRAME-expressing HEK293T cells positive for HLA-A*02:01, both in vitro and in vivo. The two target cell subsets were labeled with fluorescent dyes to enable flow cytometry analysis. The HEK293T cells used also contain granzyme B-activated infrared fluorescent protein (IFP) reporter to allow cells targeted by a TCR to become fluorescent. While using a single TCR is sub-optimal as it only targets a fraction of the tumor cells, sparing the subset of cells that cannot be recognized by the TCR, combining the two TCRs results in the killing of both cancer cell subsets simultaneously. By accumulating potent and selective therapeutic TCRs that recognize different epitopes from multiple cancer-associated proteins and address different HLA alleles in an ImmunoBank of therapeutic TCRs, treatment decisions based on the biology of the patient's tumor to create customized combinations of TCRs are enabled.

FIG. 7 shows a representative depiction of multiplex TCR-T therapeutic mechanism of action.

FIG. 8A-FIG. 8E show a representative flow cytometry gating strategy to determine TCR-T cell-mediated killing. Three representative batches of singleplex TSC-204-A0201 or TSC-204-C0702 and multiplex T-Plex-204-A0201/C0702 TCR-T cells were co-cultured with a target cell population consisting of a balanced mix of U266B1 knocked out for HLA-A*02:01 (“C7 Targets”, labeled with CFSE) and U266B1 knocked out for HLA-C*07:02 (“A2 Targets”, labeled with CellTrace™ Violet) for ˜20 hours. The residual cells from the different subset (effectors, A2 Targets and C7 Targets) in the co-culture were analyzed for cell viability by flow cytometry (shown are representative data obtained with T-Plex-204-A0201/C0702 TCR-T cells from a representative batch). Cells were gated from the FSC-A versus SSC-A dot plot (FIG. 8A) and subpopulations were distinguished using CellTrace™ CFSE versus CellTrace™ Violet plot (FIG. 8B). Viable cells for each subpopulation were identified in the histograms of LIVE/DEAD™ versus events (FIGS. 8C-8E).

FIG. 9 shows a representative depiction of viability of target cells in a heterogeneous tumor model (U266B1 HLA-A*02:01 KO combined with U266B1 HLA-C*07:02 KO) co-cultured with singleplex (TSC-204-A0201 or TSC-204-C0702) or multiplex (T-Plex-204-A0201/204-C0702) TCR-T cells. U266B1 HLA-C*07:02 KO and U266B1 HLA-A*02:01 KO target cells were fluorescently barcoded, an aliquot of each was designated for the control, and the remaining were then combined to create a balanced heterogeneous target. Three batches of single TCR-T cell agent (“singleplex”), TSC-204-A0201 or TSC-204-C0702, and multiplex, T-Plex-204-A0201/C0702 TCR-T cells were co-cultured with the heterogeneous target cells or the single target cells only (control). Barcoded target cell number and their viability were determined by flow cytometric analysis. The viable cell count of each target was normalized to absolute counting beads then graphed. Similarly, the percent viability of each target was graphed to determine T-Plex mediated killing. The left box of each pair of boxed data represents data from U266B1 HLA-C7 KO cells and the right box of each pair of boxed data represents data from U266B1 HLA-A2 KO cells.

### DETAILED DESCRIPTION OF THE INVENTION

The present invention is based, at least in part, on the discovery of certain binding proteins, including T cell receptors (TCRs), that in combination recognize more than one antigen (e.g., more than one antigen on the same target and/or more than one antigen on different targets), and engineered cells comprising same, can overcome antigen heterogeneity and/or human leukocyte antigen (HLA) loss-of-heterozygosity to treat cancers, including solid tumors.

Accordingly, the present invention relates, in part, to the identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to cells expressing antigens and/or targets of interest, and methods for preventing and/or treating a non-malignant disorder, a hyperproliferative disorder, or a relapse of a hyperproliferative disorder characterized by expression of the antigens and/or targets of interest by administering two or more binding proteins directly or compositions providing same, such as a single composition comprising two or more binding proteins, a single composition comprising nucleic acids and/or vectors encoding two or more binding proteins (e.g., TCRs), a single composition comprising a host cell type expressing two or more binding proteins (e.g., TCRs), a combination of two or more compositions each of which comprising at least one binding protein (e.g., TCRs), a combination of two or more compositions each of which comprising a nucleic acid and/or vector encoding at least one binding protein (e.g., TCR), a combination of two or more compositions each of which comprising a host cell expressing at least one binding protein (e.g., TCR), and the like. Administration may be of a single composition or a combination of compositions, either concurrently or sequentially. The two or more binding proteins may be 2, 3, 4, 5, 6, 7, 8, 9, 10, 11, 12, 13, 14, 15, 16, 17, 18, 19, 20, or more binding proteins, or any range in between, inclusive, such as 2-5 binding proteins, 2-4 binding proteins, 2-3 binding proteins, etc. In some embodiments, suitable subjects are selected using an active step of target gene expression analysis, HLA loss-of-heterozygosity (LOH), and/or HLA typing to determine compatibility with TCR binding to desired MHC:peptide (pMHC) complexes and expected therapeutic efficacy. Numerous representative, non-limiting combinations are exemplified herein and any combination of any agents described herein are contemplated compostions and uses thereof and are encompassed by the present invention.

Moreover, as described further below and in the working examples, a host ell encompassed by the present invention may encode and/or express useful accessory proteins in addition to a binding protein as described herein, either on the same polynucleotide or a different polynucleotide as the binding protein or components thereof. For example, the host cell may encode and/or express TCRα, TCRβ, CD8α, CD8β, a DN-TGFβR (e.g., a DN-TGFβRII), and/or a selectable protein marker, optionally wherein the selectable protein marker is DHFR.

The term “dominant negative TGFβ receptor” or “DN-TGFβR” refers to a transforming growth factor (TGF) beta receptor variant or mutant that provides resistance to TGFβ signaling. There are five type II receptors (activation receptors) and seven type I receptors (signaling propagation receptors). The active TGFβ receptor is a heterotetramer consisting of two TGF β receptors I (TGFβRI) and two TGF β receptors II (TGFβRII). In some embodiments, the DN-TGFβR is a DN-TGFβRII (i.e., a TGF beta receptor II variant or mutant). In some embodiments, resistance is to the suppressive effect of TGFβ signaling on an immune cell, such as a T cell, which TGFβ may be produced by cancer cells or by other immune cells within a cellular environment, such as by stromal cells, macrophages, myeloid cells, epithelial cells, natural killer cells, and the like. TGFβ signaling inhibitors are well-known in the art and include, without limitation, mutant TGFβ that sequesters receptors and thereby inhibits signaling, antibodies that bind to TGFβ and/or TGFβ receptors (e.g., lerdelimumab, metlimumab, fressolimumab, and the like), soluble TGFβ-binding proteins such as portions of TGFβ receptors that sequester TGFβ (e.g., TGFβRII-Fc fusion proteins) or other binders, such as beta-glycans. Any and all known TGFβ signaling inhibitors may be used instead of or in addition to DN-TGFβR (e.g., DN-TGFβRII) described herein. In some embodiments, a DN-TGFβR lacks an intracellular portion required for TGFβ-mediated signaling, such as the entire intracellular domain, a kinase signaling domain, etc. DN-TGFβR constructs are well-known in the art (see representative, non-limiting embodiments at Brand et al. (1993) J. Biol. Chem. 268:11500-11503; Weiser et al. (1993) Mol. Cell Biol. 13:7239-7247; Bollard et al. (2002) Blood 99: 3179-3187; PCT Publ. WO 2009/152610; PCT Publ. WO 2017/156484; Kloss et al. (2018) Mol. Ther. 26:1855-1866; PCT Publ. WO. 2019/089884; PCT Publ. WO 2020/042647; and PCT Publ. WO 2020/042648.

### EXAMPLES

### Example 1: Materials and Methods for Example 2

### A. Multiplexing HPV and MAGE-A1 TCRs

### (i) Engineering T Cells to Express HPV16-E711-19-Specific or MAGE-A1290-297 TCRs

Primary CD3+ T cells were isolated from Leukopaks using the StraightFrom® Leukopak® CD3 Microbead Kit (Miltenyi Biotec) according to manufacturer's protocol. Isolated cells were frozen in CryoStor® CS10 (Stem Cell Technologies) and stored in liquid nitrogen until use. On day −1, CD3+ T cells were thawed, washed with complete T cell medium (RPMI 1640 supplemented with 10% heat-inactivated fetal bovine serum (FBS), 100 IU/mL penicillin, 100 μg/mL streptomycin, recombinant human IL-2 [50 U/mL, PeproTech, Cranbury, NJ], recombinant human IL-15 [5 ng/mL, R&D Systems], and recombinant human IL-7 [5 ng/ml, (R&D Systems]). On day 0, CD3+ T cells were washed and resuspended in fresh T cell medium and activated using ImmunoCult™ human CD3/CD28/CD2 T cell activator (5 μL/1×10⁶ CD3+ T cells, Stem Cell Technologies). On day 1, cells were washed and resuspended in fresh complete T cell medium, and plated at 1×10⁶ cells per well. Triplicate wells were transduced with lentiviral particles to express either HPV or MAGE-A1 TCRs. On day 2, cells were washed, triplicates were resuspended and combined in fresh complete T cell medium, and expanded until day 5 in 1 well of a G-Rex® 6-well plate (Wilson Wolf). On day 5, cells were harvested, and cell concentrations were adjusted to 100×10⁶ CD3+ T cells/mL in EasySep buffer (StemCell Inc) and FcBlock solution with CD34 magnetic beads (Miltenyi) for 30 minutes at 4 C, washed with EasySep buffer, and separated using a QuadroMACS® Separator and LS columns (Miltenyi). Isolated cells were washed and resuspended in fresh complete T cell medium and expanded in G-Rex®10 flasks (Wilson Wolf) until day 12, at which point cells were frozen in CryoStor® CS10 and stored at liquid nitrogen until use.

b. Multiplexing MAGE-C2 and MAGE-A1 TCRs

### (i) Engineering T Cells to Express MAGE-C2184-192 or MAGE-A1290-297 TCRs

Primary CD8+ T cells were isolated using the StraightFrom® Leukopak® CD8 Microbead Kit (Miltenyi Biotec) according to manufacturer's protocol. Isolated cells were frozen in CryoStor® CS10 (Stem Cell Technologies) and stored in liquid nitrogen until use. On day-1, CD8+ T cells were thawed, washed with complete T cell medium (RPMI 1640 supplemented with 10% heat-inactivated fetal bovine serum (FBS), 100 IU/mL penicillin, 100 μg/mL streptomycin, recombinant human IL-2 [50 U/mL, PeproTech, Cranbury, NJ], recombinant human IL-15 [5 ng/mL, R&D Systems], and recombinant human IL-7 [5 ng/mL, (R&D Systems]). On day 0, CD8+ T cells were washed and resuspended in fresh T cell medium and activated using ImmunoCult™ human CD3/CD28/CD2 T cell activator (5 μL/1×10⁶ CD8+ T cells, Stem Cell Technologies). On day 1, cells were washed and resuspended in fresh complete T cell medium, and plated at 1×10⁶ cells per well in 9 wells. Each well was transduced with lentiviral particles to express MAGE-C2 or MAGE-A1 in triplicate or maintained as a non-transduced donor control. On day 2, cells were washed, triplicates combined, and resuspended in fresh complete T cell medium and expanded until day 5 in G-Rex®6 well plates (Wilson Wolf). On day 5, cells were harvested, and cell concentrations were adjusted to 100×10⁶ CD8+ T cells/mL in MACS® running buffer (Miltenyi) and anti-mTCR biotin antibody (BioLegend) was added a 1:50 dilution for 10 minutes at room temperature then washed with MACS® running buffer. Anti-biotin microbeads (Miltenyi) were added at 1:5 dilution and incubated for 10 minutes at room temperature. Cells were washed with MACS® running buffer and resuspended in MACS® running buffer for manual magnetic separation using a QuadroMACS® Separator and LS columns (Miltenyi). Isolated cells were washed and resuspended in fresh complete T cell medium and expanded in G-Rex®10 flasks (Wilson Wolf) until day 12, at which point cells were frozen in CryoStor® CS10 and stored at liquid nitrogen until used.

### (ii) Cell Lines

Epidermoid carcinoma cell line CaSki (ATCC CRL-1550) and melanoma cell lines A101D (ATCC CRL-7898), SK-MEL-5 (ATCC HTB-70), and A2058 (ATCC CRL-11147) were purchased from the American Type Culture Collection (ATCC, Manassas, VA). CaSki cells were cultured in RPMI 1640 containing 10% heat-inactivated FBS and 1% penicillin-streptomycin [Thermo Fisher Scientific]. A101D and A2058 cells were maintained in DMEM containing 10% heat-inactivated FBS and 1% penicillin-streptomycin [Thermo Fisher Scientific] and SK-MEL-5 cells were cultured in EMEM containing 10% heat-inactivated FBS and 1% penicillin-streptomycin [Thermo Fisher Scientific].

(iii) Generation of Stable Cell Lines Expressing Incucyte® Nuclight Red

CaSki, A101D, and SK-MEL-5 cells were transduced with Incucyte® NucLight Red Lentivirus Reagent (EF-1α promoter, puromycin selection) (Sartorius). 24 hours post-transduction, cells were washed and resuspended in their respective cell line media and cultured at 37° C., 5% CO₂. 2-3 days post-transduction, puromycin (Gibco, Waltham, MA) was added to the cultures at a pre-determined concentration (ranging from 0.5 μg/mL to 1 μg/mL) to select for transduced cells. Cultures were expanded under puromycin selection until they were at least 90% Incucyte® Nuclight Red-positive as determined by flow cytometric analysis.

### (iv) In Vitro Cytotoxicity Assay

In vitro cytotoxicity assays were performed in 96-well flat-bottom tissue culture plates without coating with poly-L-ornithine; here the adherent cells were plated and allowed to attach the day before T cells were added. Where indicated, T cells were co-cultured with Incucyte® Nuclight Red-expressing CaSki, A101D, or SK-MEL-5 cells at indicated E:T ratios. Data were acquired on an Incucyte® S3 instrument (Sartorius), and target cell growth was quantified on the Incucyte® S3 as a readout of T cell cytotoxicity.

### (v) Transwell T-Cell Activation Assay

Corning® HTS Transwell®-96 Permeable Support with 1.0-μm pore polycarbonate membrane inserts (Sigma-Aldrich #CLS3392) were used according to manufacturer's instructions. A101D melanoma cells were seeded in the upper chamber, while SK-MEL-5 melanoma cells were seeded in the lower chamber and both lines were allowed to adhere overnight. The next day, CD8+ T-cells engineered with the MAGEA1 TCR were co-cultured with A101D cells in the upper chamber, while CD8+ T-cells engineered with the MAGEC2 TCR were co-cultured with SK-MEL-5 cells in the lower chamber at a 1:2 E:T ratio and incubated for 48 hours at 5% CO₂ at 37° C. Following incubation, cells were collected for evaluation by staining with antibodies against T-cell activation markers. In brief, T cells were stained with PE-labeled anti-CD137 and AF647-labeled anti-CD69 (BioLegend), washed, and then analyzed for CD137 and CD69 double-positive cells on a CytoFLEX flow cytometer (Beckman Coulter).

### Example 2: Representative, Non-Limiting Combination Therapy Example

Adoptive cell transfer with genetically engineered T cells holds great promise for treating solid tumors. To date, clinical investigations of TCR-engineered T cell therapies have targeted one antigen at a time and have produced encouraging response rates ranging from 30-50%. Unfortunately, complete responses have been rare, and responses are often short-lived. It is believed that there are two main challenges associated with single-antigen targeted TCR-T cell therapy.

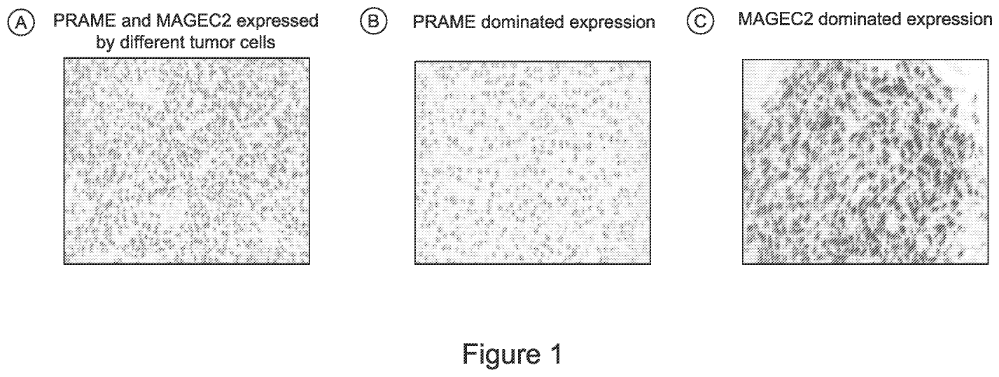

First, expression of most cancer associated antigens is heterogeneous. In one representative, non-limiting example, multiplexed immunohistochemistry was performed with MAGE-C2 and PRAME, two cancer germline antigens, and considerable heterogeneity across samples from different solid tumor types was observed (FIGS. 1A-1C). Additionally, heterogenous antigen expression was observed at the single cell level—not every cancer cell within a given tumor expressed each antigen (FIGS. 1A-1C). This indicates that a single TCR may not be sufficient to eliminate all cancer cells within a given tumor, thereby allowing the tumor cells lacking the treated antigen to escape and drive relapse.

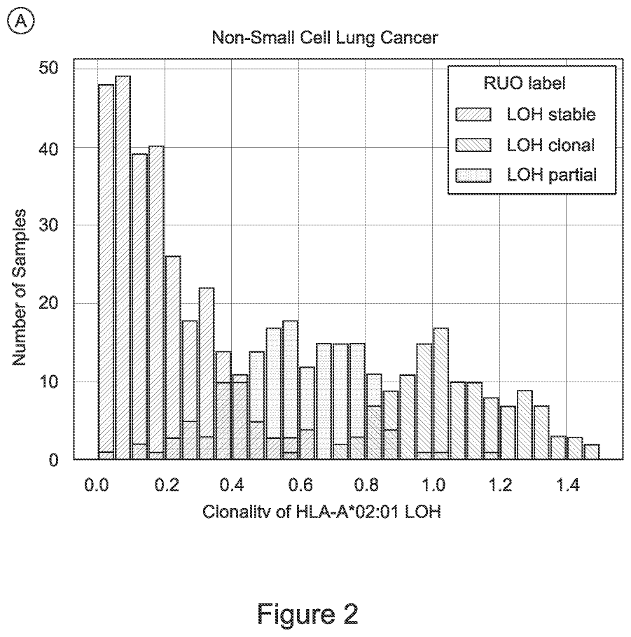

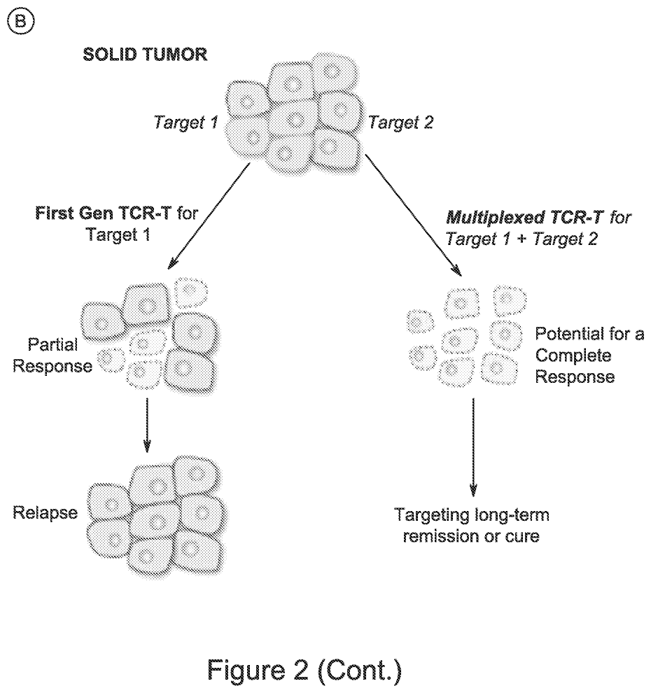

Second, single agent TCR-T cell therapy targets only a single HLA allele, which is subject to loss through commonly observed HLA loss-of-heterozygosity (LOH) mechanisms (FIGS. 2A and 2B). Clonal HLA Class I LOH has been observed in 17% of all solid tumors (Montesion et al., Cancer Discovery, 2021) and sub-clonal HLA LOH occurs in an even larger percentage of tumors.

Multiplexed TCR-T cell therapy mimics the natural oligoclonal T cell response to cancer and provides a way to address both challenges associated with treating solid tumors.

Using TScan's proprietary ReceptorScan and TargetScan platforms, a variety of TCRs, such as HPV16 E7-specific, MAGEA1-specific, and MAGEC2-specific TCRs were discovered for TCR-T cell therapy.

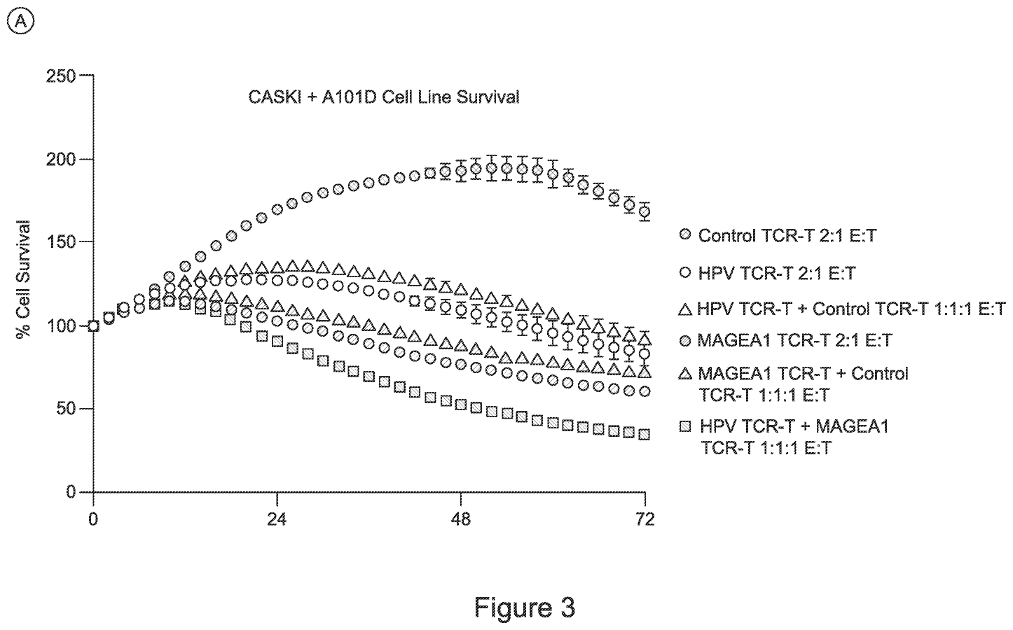

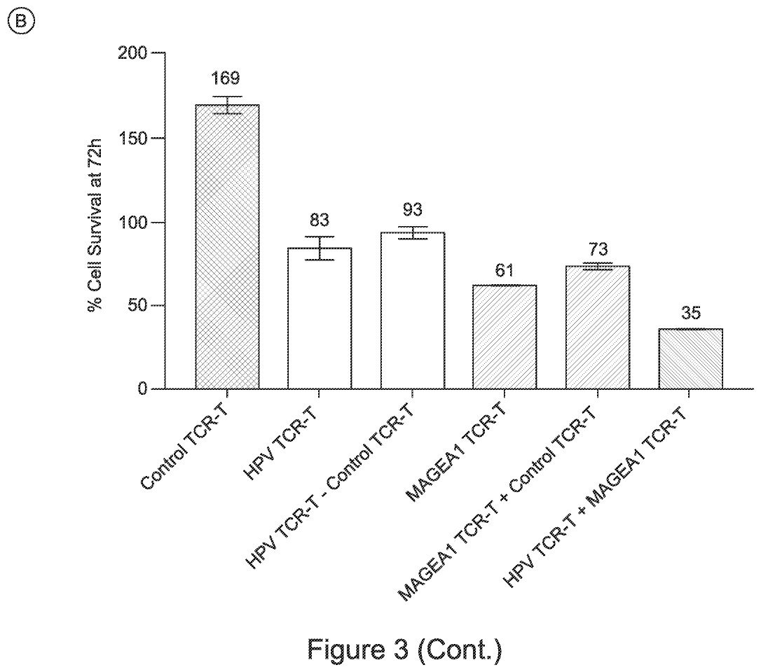

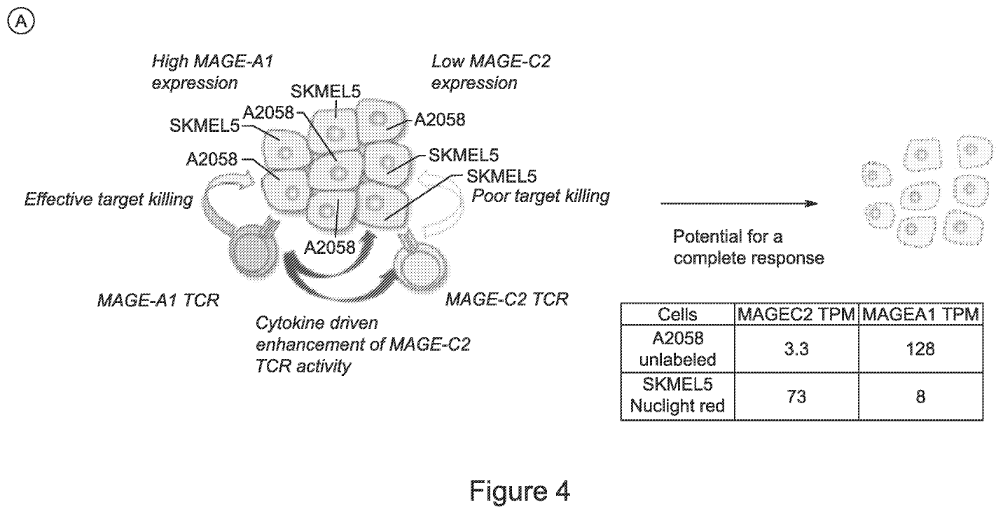

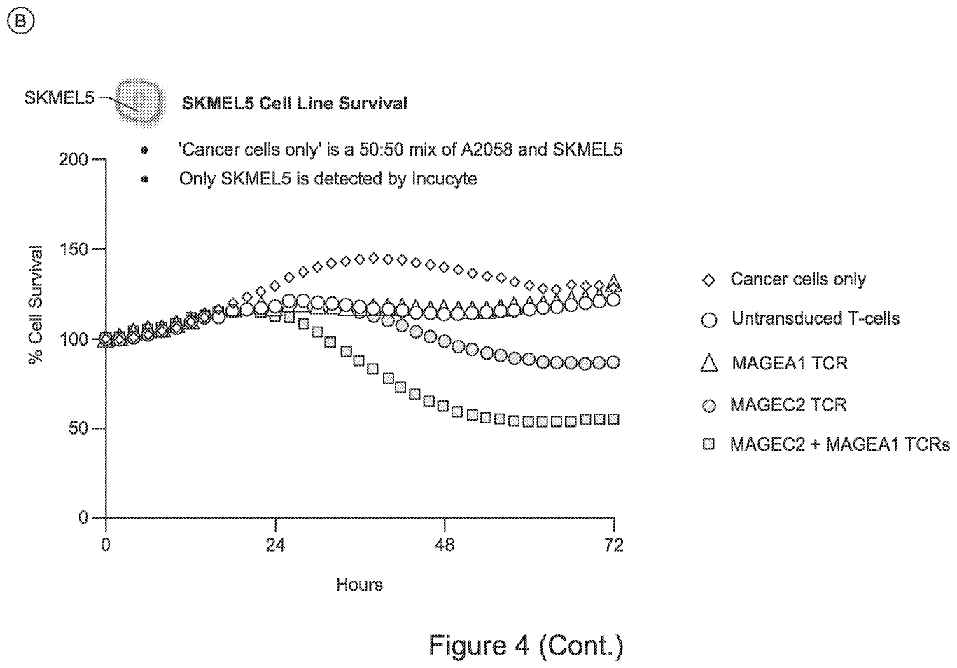

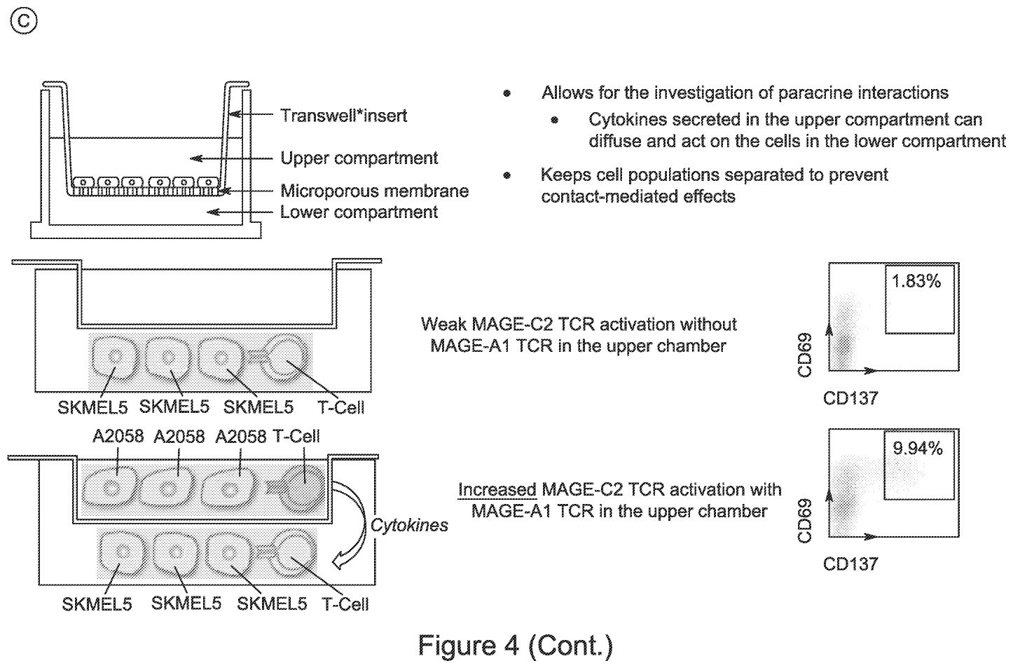

In a representative, non-limiting example, two lead TCRs (MAGE-A1 and HPV), a lower-affinity TCR (MAGE-C2), and target cell lines expressing their cognate antigens were multiplexed using direct and indirect co-culture experiments to evaluate the potential synergy of using more than one TCR to target tumors as well as understanding the biological mechanisms behind such synergy. Materials and methods, as well as results, are shown in FIGS. 1A-5B. Briefly, two distinct TCR:antigen pairs were used to model multiplexed T cell-mediated cancer killing and heterogeneity in vitro. In addition, the vector used to generate the data shown in FIG. 3 expresses a CD8 co-receptor, while the vector used to generate the data shown in FIG. 4 did not express a CD8 co-receptor, although it has the mouse TCR constant region.

In one representative case, multiplexing of two high-affinity TCR-Ts (i.e., one named TCR E7-11-28 (also known as TCR28 or 28; see Table 1) targeting an HLA-A*02:01-restricted epitope of HPV16-E7 and the second named TCR-204-C07 (also known as TCR 32-41, TCR-204-C7, and TCR-204-C0702; see Table 1) targeting an HLA-C*07:02-restricted epitope of MAGE-A1 were tested. Pan-T cells were transduced and selected to express the relevant TCRs (HPV or MAGE-A1) and, in some cases, a CD8 co-receptor. Target cells were a mixture of two cell lines, each expressing only one of the two antigens. CaSki cervical cancer cells are A*02:01+ and HPV+. A101D melanoma cells are C*07: 02+ and MAGE-A1+. Both cell lines were engineered to express Incucyte®NucLight Red and mixed together to mimic tumor heterogeneity. Engineered T cells or non-engineered donor control T-cells (Control TCR-T) were co-cultured with Incucyte® NucLight Red-labeled target cell lines at indicated effector cell to target cell (E:T) ratios, and their survival was quantified on an IncuCyte® as a readout of cytotoxicity of the T cells. Whereas individual TCR-Ts caused ˜50-60% cell killing at 72 h, a 1:1 mix of the two TCR-Ts resulted in ˜80% cell killing at the same overall effector to target (E:T) ratio, indicating a synergistic effect (FIGS. 3A and 3B).

In another representative case, multiplexing a high affinity TCR-T for MAGE-A1 with a low-affinity TCR-T for MAGE-C2 was tested. TCR-204-C07 (also known as TCR 32-41, TCR-204-C7, and TCR-204-C0702; see Table 1) is a naturally occurring, high affinity TCR that recognizes an HLA-C*07:02-restricted epitope of MAGEA1 and exhibits robust killing of cell lines expressing MAGEA1. TCR-LD8-3 is a low affinity TCR that recognizes an HLA-B*07:02-restricted epitope of MAGEC2 (also known as TCR 8-3; see Table 1) (FIG. 4A). Lentiviral vectors encoding HM codon-optimized TCRs for better expression were used to transduce CD8+ T-cells. Transduced cells were selected based on the expression of relevant TCRs (MAGE-A1 or MAGE-C2). The target cells were a mixture of MAGE-A1- or MAGE-C2-expressing cells. A2058 and SK-MEL-5 cells are both melanoma cell lines that are both C*07: 02+ and B*07: 02+, however A2058 cells only highly express MAGE-A1 and SK-MEL-5 cells only moderately express MAGE-C2. It was previously found that, although the MAGE-C2 TCR-T cells effectively kill A101D cells that express MAGEC2 at high levels, it is ineffective at killing SK-MEL-5 cells, which express MAGEC2 at lower levels. To determine if the cytotoxic activity of the MAGE-C2 TCR-T cells could be enhanced when multiplexed with a more cytotoxic TCR, a co-culture experiment was performed. SK-MEL-5 cells engineered to express Incucyte® NucLight Red were mixed together with unlabeled A2058 cells such that the activity quantified on an IncuCyte® would reflect only the cytotoxicity towards SK-MEL-5 cells. Engineered T cells or non-engineered donor control T-cells (Untransduced T-cells) were co-cultured with target cell lines at indicated effector cell to target cell (E:T) ratios, and their survival was quantified on an IncuCyte® as a readout of cytotoxicity of the T cells. While a low-level killing of SK-MEL-5 was observed by the single MAGE-C2 T-cells, when MAGE-C2 and MAGE-A1 T-cells were combined, the cytotoxic activity of the MAGE-C2 TCR was synergistically enhanced. Thus, although the MAGE-C2 TCR-T alone displayed partial killing of MAGE-C2-positive cells, addition of the MAGE-A1 TCR-T synergistically enhanced the activity of the MAGE-C2 TCR-T (FIG. 4B).

To explore the mechanism of this synergistic activity, a Corning® HTS Transwell® system was used. HTS Transwell-96 Permeable Support with 1.0 μm pore polyester membrane transwell system was selected to allow for diffusion of soluble factors, but not cells, between the two cellular compartments; an upper chamber and a lower chamber. A101D melanoma cells were seeded in the upper chamber while SK-MEL-5 melanoma cells were seeded in the lower chamber and both lines were allowed to adhere overnight. The next day, CD8+ T-cells engineered with the MAGEA1 TCR were co-cultured with A101D cells in the upper chamber while CD8+ T-cells engineered with the MAGEC2 TCR were co-cultured with SK-MEL-5 cells in the lower chamber at a 1:2 E:T ratio. After 48 hours, the cells from either chamber were collected for evaluation by staining with antibodies against T-cell activation markers. In brief, T cells were stained with PE-labeled anti-CD137 and AF647-labeled anti-CD69 (BioLegend), washed, and then analyzed for CD137 and CD69 double-positive cells on a CytoFLEX flow cytometer (Beckman Coulter). Using the transwell culturing system, it was found that cytokines secreted by MAGE-A1 TCR-Ts strongly enhanced T cell activation of MAGE-C2 TCR-T cells upon antigen engagement (FIG. 4C). These findings indicate that multiplexed TCR-T may overcome antigen heterogeneity not only through independent targeting of different cancer cell populations, but also by cytokine-mediated T cell enhancement. Surprisingly, these results demonstrate unexpectedly synergistic effect.

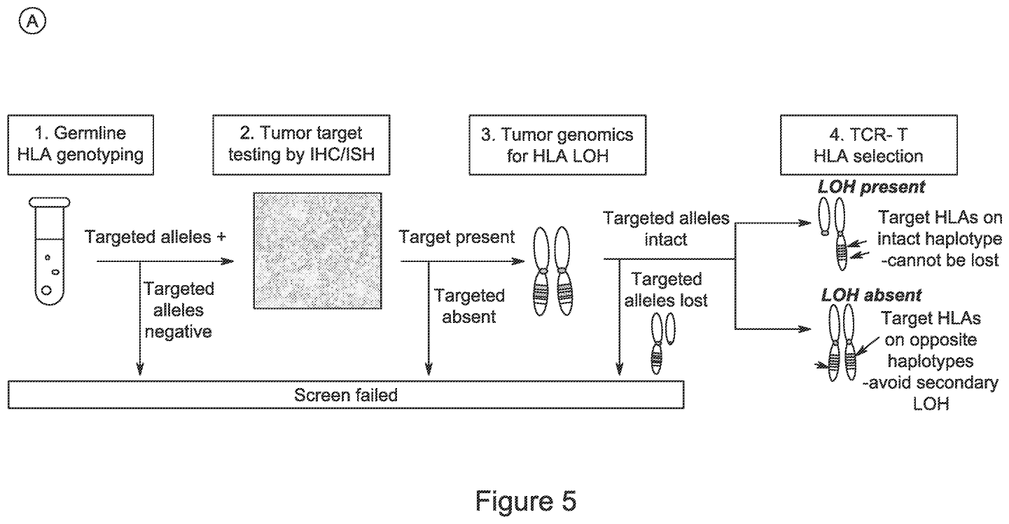

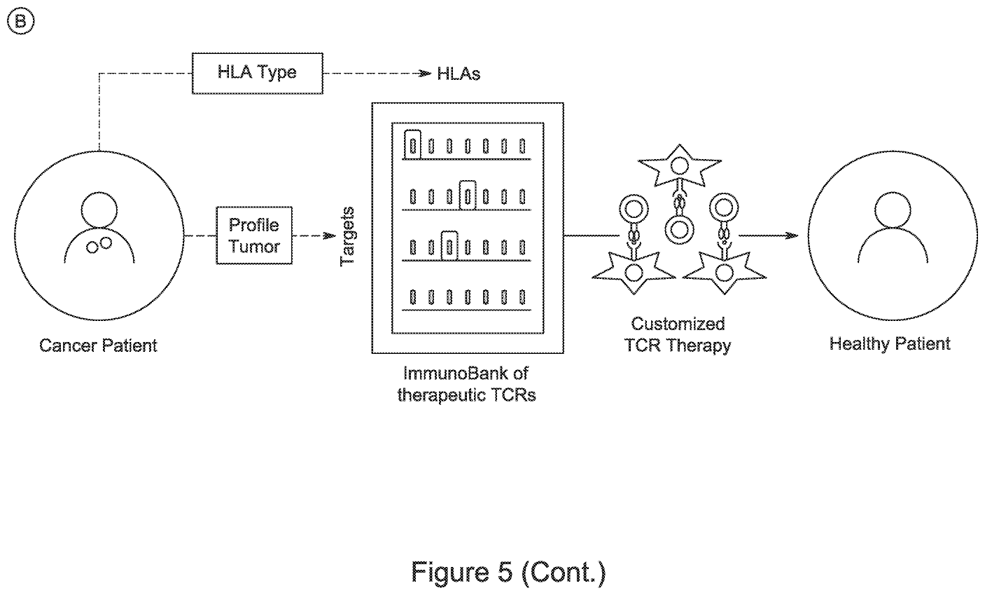

These results have been adapted for clinical applications. For example, in order to address solid tumor heterogeneity in the clinic, an illustrative screening strategy was designed to test patient tumors for antigen positivity and HLA LOH (FIG. 5A). In addition, an ImmunoBank of therapeutic TCRs that recognize different targets presented on different HLA alleles. Selecting multiplexed TCR-Ts that target intact antigens and HLA alleles in patient tumors is believed to synergistically overcome solid tumor heterogeneity. Additional in vivo studies are being conducted to further confirm the synergistic effect of multiplexed TCR-T cell therapy and clinical trials are being designed to further confirm the syntergistic effects clinically.

Thus, the present Example provides compositions and methods useful for multiplexed TCR-T cell therapy, including a combination of anti-MAGE-A1 and anti-HPV TCRs or a combination of an anti-MAGE-A1 and anti-MAGE-C2 TCRs, and engineered cells expressing same. Without wishing to be bound by any particular scientific theory, the present Example further includes that multiplexed TCR-T cell therapy mimics a natural oligoclonal T cell response to cancer. Multiplexed TCR-T cell therapy, such as a combination described above, provides for methods and compositions that address certain challenges associated with treating solid tumors.

Various assays can be used to confirm the utility of a multiplexed TCR-T cell therapy, such as a combination described above. In a representative, non-limiting example, a combination of anti-MAGE-A1 and anti-HPV TCRs or a combination of an anti-MAGE-A1 and anti-MAGE-C2 TCRs, and one or more target cell lines expressing their cognate antigens are multiplexed using direct and indirect co-culture experiments to evaluate the potential synergy of using more than one TCR to target tumors, as well as understanding the biological mechanisms behind such synergy. This assay can be used to model multiplexed T cell-mediated cancer killing by a multiplexed TCR-T cell therapy that includes the TCR combinations of interest, and heterogeneity, in vitro.

In one representative case, multiplexing of (i) an anti-MAGE-A1 TCR targeting an HLA-C*07 serotype-restricted epitope of MAGE-A1 (Table 3A) and (ii) an anti-HPV16 E7 TCR targeting an HLA-A*02 serotype-restricted epitope of HPV16 E7 (Table 3C), such as by using engineered cells expressing such TCRs, can be used and/or tested.

In another representative case, multiplexing of (i) an anti-MAGE-A1 TCR targeting an HLA-C*07 serotype-restricted epitope of MAGE-A1 (Table 3A) and (ii) an anti-MAGE-C3 TCR targeting an HLA-B*07 serotype-restricted epitope of MAGE-C3 (Table 3B), such as by using engineered cells expressing such TCRs, can be used and/or tested.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in a Table provided herein; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of): a) a TCR alpha chain sequence selected from the group consisting of the TCR alpha chain sequences listed in a Table provided herein; and/or b) a TCR beta chain sequence selected from the group consisting of the TCR beta chain sequences listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in a Table provided herein; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR VB domain sequences listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of): a) a TCR alpha chain variable (V_{α}) domain sequence selected from the group consisting of the TCR V_{α} domain sequences listed in a Table provided herein; and/or b) a TCR beta chain variable (V_{β}) domain sequence selected from the group consisting of the TCR VB domain sequences listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of): at least one (e.g., one, two or three, such as CDR3 alone or in combination with a CDR1 and CDR2) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain CDR sequence selected from the group consisting of the TCR alpha chain CDR sequences listed in Table provided herein. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in a Table provided herein, each CDR3 having a sequence homology as recited in this present Example, are provided.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) at least one (e.g., one, two or three, such as CDR3 alone or in combination with a CDR1 and CDR2) TCR beta chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain CDR sequence selected from the group consisting of the TCR beta chain CDR sequences listed in a Table provided herein. As described above, CDR3 is believed to be the main CDR responsible for recognizing processed antigen and CDR1 and CDR2 mainly interact with the MHC, so, in some embodiments, a binding protein comprising a CDR3 alone from a TCR beta chain and/or a CDR3 alone from a TCR alpha chain listed in a Table provided herein, each CDR3 having a sequence homology as recited in this Example, are provided.

An anti-MAGE-A1 TCR, anti-HPV TCR, anti-MAGE-C2 TCR, and/or anti-PRAME TCR, either alone or in any combination thereof, TCR encompassed by the present invention can be a TCR that includes (e.g., comprises, consist essentially of, or consists of) at least one (e.g., one, two or three)) TCR alpha chain complementarity determining region (CDR) listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) at least one (e.g., one, two or three)) TCR beta chain complementarity determining region (CDR) listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) a TCR alpha chain constant region (Ca) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR Ca sequence listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) a TCR beta chain constant region (CB) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) a TCR alpha chain constant region (C_{α}) sequence selected from the group consisting of the TCR C_{α} sequences listed in a Table provided herein.

An anti-MAGE-A1 TCR, anti-HPV TCR, anti-MAGE-C2 TCR, and/or anti-PRAME TCR, either alone or in any combination thereof, encompassed by the present invention can be a TCR that includes (e.g., comprises, consist essentially of, or consists of) a TCR beta chain constant region (C_{β}) sequence selected from the group consisting of the TCR C_{β} sequences listed in a Table provided herein.

TABLE 1
Representative TCR sequences

| | |
|---|---|
| MAGEA1 | Alpha chain DNA sequence |
| TCR 32-41 | ATGGTCCTGAAATTCTCCGTGTCCATTCTTTGGATTCAGTTGGCA |
| wild type | TGGGTGAGCACCCAGCTGCTGGAGCAGAGCCCTCAGTTTCTTAG |
| sequence | CATCCAAGAGGGAGAAAATCTCACTGTGTACTGCAACTCCTCAA |
| Alpha chain: | GTGTTTTCTCCAGCCTTCAATGGTACAGACAGGAGCCTGGGGA |
| TRAV27/ | AGGTCCTGTCCTCCTGGTGACAGTTGTTACTGGTGGAGAAGTG |
| TRAJ52/TRAC | AAGAAGCTGAAGAGACTTACCTTTCAGTTTGGTGATGCAAGAAA |
| | GGACAGTTCTCTCCACATCACTGCAGCCCAGCCTGGTGATACAG |
| | GCCTCTACCTCTGTGCAGGAGATGAAAGTATTAGCTATGGAA |
| | AGCTGACATTTGGACAAGGGACCATCTTGACTGTCCATCCAAata |
| | tccagaaccctgaccctgccgtgtaccagctgagagactctaaatccagtgacaagtctgtctgcctattca |
| | ccgattttgattctcaaacaaatgtgtcacaaagtaaggattctgatgtgtatatcacagacaaaactgtgcta |
| | gacatgaggtctatggacttcaagagcaacagtgctgtggcctggagcaacaaatctgactttgcatgtgca |
| | aacgccttcaacaacagcattattccagaagacaccttcttccccagcccagaaagttcctgtgatgtcaag |
| | ctggtcgagaaaagctttgaaacagatacgaacctaaactttcaaaacctgtcagtgattgggttccgaatcc |
| | tcctcctgaaagtggccgggtttaatctgctcatgacgctgcggctgtggtccagc (SEQ ID NO: 1) |
| | Alpha chain protein sequence |
| | MVLKFSVSILWIQLAWVSTQLLEQSPQFLSIQEGENLTVYCNSSSVF |
| | SSLQWYRQEPGEGPVLLVTVVTGGEVKKLKRLTFQFGDARKDSSL |
| | HITAAQPGDTGLYLCAGDESISYGKLTFGQGTILTVHPNiqnpdpavyq |
| | lrdskssdksvclftdfdsqtnvsqskdsdvyitdktvldmrsmdfksn |
| | savawsnksdfacanafnnsiipedtffpspesscdvklveksfetdtn |
| | lnfqnlsvigfrilllkvagfnllmtlrlwss (SEQ ID NO: 2) |
| MAGEA1 | Beta chain DNA sequence |
| TCR 32-41 | ATGGGCTCCTGGACCCTCTGCTGTGTGTCCCTTTGCATCCTGGTT |
| wild type | GCAAAGCACACAGATGCTGGAGTTATCCAGTCACCCCGGCACGA |
| sequence | GGTGACAGAGATGGGACAAGAAGTGACTCTGAGATGTAAACCA |
| Beta chain: | ATTTCAGGACATGACTACCTTTTCTGGTACAGACAGACCATGAT |
| TRBV12-4/ | GCGGGGACTGGAGTTGCTCATTTACTTTAACAACAACGTTCCT |
| TRBJ1-4/ | ATTGATGATTCAGGGATGCCCGAGGATCGCTTCTCAGCTAAGAT |
| TRBC1 | GCCTAATGCATCATTCTCCACTCTGAAGATCCAGCCCTCAGAAC |
| | CCAGGGACTCAGCTGTGTACTTCTGTGCCAGCAGTTTTCTCGG |
| | CTGGAATGAAAAACTGTTCTTTGGCAGTGGAACCCAGCTCTCT |
| | GTCTTGGaggacctgaacaaggtgttcccacccgaggtcgctgtgtttgagccatcagaagcagag |
| | atctcccacacccaaaaggccacactggtgtgcctggccacaggcttcttccctgaccacgtggagctgag |
| | ctggtgggtgaatgggaaggaggtgcacagtggggtcagcacggacccgcagcccctcaaggagcag |
| | cccgccctcaatgactccagatactgcctgagcagccgcctgagggtctcggccaccttctggcagaacc |
| | cccgcaaccacttccgctgtcaagtccagttctacgggctctcggagaatgacgagtggacccaggatag |
| | ggccaaacccgtcacccagatcgtcagcgccgaggcctggggtagagcagactgtggctttacctcggt |
| | gtcctaccagcaaggggtcctgtctgccaccatcctctatgagatcctgctagggaaggccaccctgtatgc |
| | tgtgctggtcagcgcccttgtgttgatggccatggtcaagagaaaggatttc (SEQ ID NO: 3) |
| | Beta chain protein sequence |
| | MGSWTLCCVSLCILVAKHTDAGVIQSPRHEVTEMGQEVTLRCKPIS |
| | GHDYLFWYRQTMMRGLELLIYFNNNVPIDDSGMPEDRFSAKMPN |
| | ASFSTLKIQPSEPRDSAVYFCASSFLGWNEKLFFGSGTQLSVLEdlnk |
| | vfppevavfepseaeishtqkatlvclatgffpdhvelswwvngkevh |
| | sgvstdpqplkeqpalndsryclssrlrvsatfwqnprnhfrcqvqfy |
| | glsendewtqdrakpvtqivsaeawgradcgftsvsyqqgvlsatily |
| | eillgkatlyavlvsalvlmamvkrkdf (SEQ ID NO: 4) |
| MAGEA1 | Alpha chain DNA sequence |
| TCR 32-41 | ATGGTCCTGAAATTCTCCGTGTCCATTCTTTGGATTCAGTTGGCA |
| HM codon | TGGGTGAGCACCCAGCTGCTGGAGCAGAGCCCTCAGTTTCTTAG |
| optimized | CATCCAAGAGGGAGAAAATCTCACTGTGTACTGCAACTCCTCAA |
| sequence | GTGTTTTCTCCAGCCTTCAATGGTACAGACAGGAGCCTGGGGA |
| Note: This | AGGTCCTGTCCTCCTGGTGACAGTTGTTACTGGTGGAGAAGTG |
| clone was used | AAGAAGCTGAAGAGACTTACCTTTCAGTTTGGTGATGCAAGAAA |
| in FIG. 4 as | GGACAGTTCTCTCCACATCACTGCAGCCCAGCCTGGTGATACAG |
| MAGEA1 | GCCTCTACCTCTGTGCAGGAGATGAAAGTATTAGCTATGGAA |
| TCR in | AGCTGACATTTGGACAAGGGACCATCTTGACTGTCCATCCAAac |
| multiplex with | attcaaaacccagaacccgccgtctaccagctgaaagacccgaggtctcaagactctacgitgtgcttgttc |
| the MAGEC2 | accgatttcgacagtcagataaatgtgcctaagaccatggagagtggcactttcatcactgacaaatgtgtgt |
| TCR. Also, a | tggacatgaaggctatggacagcaagtcaaacggcgcgattgcttggtccaaccaaacttctttcacgtgcc |
| TCR-alone | aggacatcttcaaggagacaaacgccacctatccatcctctgatgttccgtgcgatgcgactcttaccgaga |
| vector was | aaagcttcgagacggacatgaacttgaacttccaaaacctgcttgtgatggtactgcgaatacttcttcttaag |
| used for this | gtggcgggcttcaatttgctcatgacactcagactttggtctagc (SEQ ID NO: 5) |
| experiment | Alpha chain protein sequence |
| such that no | MVLKFSVSILWIQLAWVSTQLLEQSPQFLSIQEGENLTVYCNSSSVF |
| additional | SSLQWYRQEPGEGPVLLVTVVTGGEVKKLKRLTFQFGDARKDSSL |
| elements (e.g., | HITAAQPGDTGLYLCAGDESISYGKLTFGQGTILTVHPNiqnpepavyq |
| CD8alpha/beta | lkdprsqdstlclftdfdsqinvpktmesgtfitdkcvldmkamdsksn |
| co-receptor, | gaiawsnqtsftcqdifketnatypssdvpcdatlteksfetdmnlnfq |
| DN-TGFBRII, | nllvmvlrilllkvagfnllmtlrlwss (SEQ ID NO: 6) |
| and the like | |
| were not | |
| expressed). | |
| Data shown in | |
| FIG. 3 were | |
| generated | |
| using the HM | |
| version of the | |
| sequence. | |
| Alpha chain: | |
| TRAV27/ | |
| TRAJ52/codon | |
| optimized | |
| mouse TRAC | |
| MAGEA1 | Beta chain DNA sequence |
| TCR 32-41 | ATGGGCTCCTGGACCCTCTGCTGTGTGTCCCTTTGCATCCTGGTT |
| HM codon | GCAAAGCACACAGATGCTGGAGTTATCCAGTCACCCCGGCACGA |
| optimized | GGTGACAGAGATGGGACAAGAAGTGACTCTGAGATGTAAACCA |
| sequence | ATTTCAGGACATGACTACCTTTTCTGGTACAGACAGACCATGAT |
| Note: This | GCGGGGACTGGAGTTGCTCATTTACTTTAACAACAACGTTCCT |
| clone was used | ATTGATGATTCAGGGATGCCCGAGGATCGCTTCTCAGCTAAGAT |
| in FIG. 4 as | GCCTAATGCATCATTCTCCACTCTGAAGATCCAGCCCTCAGAAC |
| MAGEA1 | CCAGGGACTCAGCTGTGTACTTCTGTGCCAGCAGTTTTCTCGG |
| TCR in | CTGGAATGAAAAACTGTTCTTTGGCAGTGGAACCCAGCTCTCT |
| multiplex with | GTCTTGGaagatcttcgaaacgtaacccctccaaaagtgagtctctttgaaccgagtaaggctgagat |
| the MAGEC2 | cgcgaacaaacaaaaggcgaccctcgtctgtcttgcgcgaggattttttcccgaccacgtggagttgtcttg |
| TCR. Also, a | gtgggtaaacggtaaggaagtacacagcggtgtttgcaccgaccctcaagcctacaaggaatctaactatt |
| TCR-alone | catactgcctttcatcccgacttagggtttctgctaccttttggcacaatccgaggaatcactttaggtgtcaag |
| vector was | tacagttccacggattgtcagaggaggataaatggccggagggctccccgaagccggttacgcagaacat |
| used for this | tagtgcggaagcctggggacgagcagactgcggtatcacgtctgccagctatcagcaaggcgttctgtca |
| experiment | gcgacaattctgtacgaaatacttttgggtaaggctacattgtatgcggtattggtgtctacgctggtagtcatg |
| such that no | gccatggtgaaacgaaaaaactca (SEQ ID NO: 7) |
| additional | Beta chain protein sequence |
| elements (e.g., | MGSWTLCCVSLCILVAKHTDAGVIQSPRHEVTEMGQEVTLRCKPIS |
| CD8alpha/beta | GHDYLFWYRQTMMRGLELLIYFNNNVPIDDSGMPEDRFSAKMPN |
| co-receptor, | ASFSTLKIQPSEPRDSAVYFCASSFLGWNEKLFFGSGTQLSVLEdlrn |
| DN-TGFBRII, | vtppkvslfepskaeiankqkatlvclargffpdhvelswwvngkevh |
| and the like | sgvctdpqaykesnysyclssrlrvsatfwhnprnhfrcqvqfhglse |
| were not | edkwpegspkpvtqnisaeawgradcgitsasyqqgvlsatilyeill |
| expressed). | gkatlyavlvstlvvmamvkrkns (SEQ ID NO: 8) |
| Data shown in | |
| FIG. 3 were | |
| generated | |
| using the HM | |
| version of the | |
| sequence. | |
| Beta chain: | |
| TRBV12-4/ | |
| TRBJ1-4/ | |
| codon | |
| optimized | |
| mouse TRBC | |
| Complete Beta | ATGGGCTCCTGGACCCTCTGCTGTGTGTCCCTTTGCATCCTGGTT |
| and Alpha | GCAAAGCACACAGATGCTGGAGTTATCCAGTCACCCCGGCACGA |
| ORF DNA | GGTGACAGAGATGGGACAAGAAGTGACTCTGAGATGTAAACCA |
| Sequence (The | ATTTCAGGACATGACTACCTTTTCTGGTACAGACAGACCATGAT |
| underlined | GCGGGGACTGGAGTTGCTCATTTACTTTAACAACAACGTTCCT |
| italic region in | ATTGATGATTCAGGGATGCCCGAGGATCGCTTCTCAGCTAAGAT |
| the “Furin- | GCCTAATGCATCATTCTCCACTCTGAAGATCCAGCCCTCAGAAC |
| P2A” site | CCAGGGACTCAGCTGTGTACTTCTGTGCCAGCAGTTTTCTCGG |
| encodes a | CTGGAATGAAAAACTGTTCTTTGGCAGTGGAACCCAGCTCTCT |
| sequence | GTCTTGGaagatcttcgaaacgtaacccctccaaaagtgagtctctttgaaccgagtaaggctgagat |
| allowing for | cgcgaacaaacaaaaggcgaccctcgtctgtcttgcgcgaggattttttcccgaccacgtggagttgtcttg |
| expression of | gtgggtaaacggtaaggaagtacacagcggtgtttgcaccgaccctcaagcctacaaggaatctaactatt |
| two | catactgcctttcatcccgacttagggtttctgctaccttttggcacaatccgaggaatcactttaggtgtcaag |
| polypeptide | tacagttccacggattgtcagaggaggataaatggccggagggctccccgaagccggttacgcagaacat |
| chains in a | tagtgcggaagcctggggacgagcagactgcggtatcacgtctgccagctatcagcaaggcgttctgtca |
| single | gcgacaattctgtacgaaatacttttgggtaaggctacattgtatgcggtattggtgtctacgctggtagtcatg |
| cassette”) | gccatggtgaaacgaaaaaactcaAGAGCCAAAAGAAGCGGGAGCGGTGCGAC |
| | AAACTTTAGCCTGTTGAAACAAGCCGGCGACGTTGAAGAGAACCCCG |
| | GACCTATGGTCCTGAAATTCTCCGTGTCCATTCTTTGGATTCAGT |
| | TGGCATGGGTGAGCACCCAGCTGCTGGAGCAGAGCCCTCAGTTT |
| | CTTAGCATCCAAGAGGGAGAAAATCTCACTGTGTACTGCAACTC |
| | CTCAAGTGTTTTCTCCAGCCTTCAATGGTACAGACAGGAGCCT |
| | GGGGAAGGTCCTGTCCTCCTGGTGACAGTTGTTACTGGTGGAG |
| | AAGTGAAGAAGCTGAAGAGACTTACCTTTCAGTTTGGTGATGCA |
| | AGAAAGGACAGTTCTCTCCACATCACTGCAGCCCAGCCTGGTGA |
| | TACAGGCCTCTACCTCTGTGCAGGAGATGAAAGTATTAGCTAT |
| | GGAAAGCTGACATTTGGACAAGGGACCATCTTGACTGTCCATC |
| | CAAacattcaaaacccagaacccgccgtctaccagctgaaagacccgaggtctcaagactctacgttgt |
| | gcttgttcaccgatttcgacagtcagataaatgtgcctaagaccatggagagtggcactttcatcactgacaa |
| | atgtgtgttggacatgaaggctatggacagcaagtcaaacggcgcgattgcttggtccaaccaaacttctttc |
| | acgtgccaggacatcttcaaggagacaaacgccacctatccatcctctgatgttccgtgcgatgcgactctt |
| | accgagaaaagcttcgagacggacatgaacttgaacttccaaaacctgcttgtgatggtactgcgaatactt |
| | cttcttaaggtggcgggcttcaatttgctcatgacactcagactttggtctagc (SEQ ID NO: 9) |
| Complete Beta | MGSWTLCCVSLCILVAKHTDAGVIQSPRHEVTEMGQEVTLRCKPIS |
| and Alpha | GHDYLFWYRQTMMRGLELLIYFNNNVPIDDSGMPEDRFSAKMPN |
| ORF Protein | ASFSTLKIQPSEPRDSAVYFCASSFLGWNEKLFFGSGTQLSVLEdlrn |
| Sequence (The | vtppkvslfepskaeiankqkatlvclargffpdhvelswwvngkevhsgvctdpqaykesnysycls |
| underlined | srlrvsatfwhnprnhfrcqvqfhglseedkwpegspkpvtqnisaeawgradcgitsasyqqgvlsat |
| italic region in | ilyeillgkatlyavlvstlvvmamvkrknsRAKRSGSGATNFSLLKQAGDVEENPGP |
| the “Furin- | MVLKFSVSILWIQLAWVSTQLLEQSPQFLSIQEGENLTVYCNSSSVF |
| P2A” site | SSLQWYRQEPGEGPVLLVTVVTGGEVKKLKRLTFQFGDARKDSSL |
| allows | HITAAQPGDTGLYLCAGDESISYGKLTFGQGTILTVHPNiqnpepavyq |
| expression of | lkdprsqdstlclftdfdsqinvpktmesgtfitdkcvldmkamdsksngaiawsnqtsftcqdifketn |
| two | atypssdvpcdatlteksfetdmnlnfqnllvmvlrilllkvagfnllmtlrlwss (SEQ ID NO: |
| polypeptide | 10) |
| chains in a | |
| single | |
| cassette”)”) | |

Note: CDR1 and CDR2 sequences for each clone below are redundant for the corresponding
base clone name listed above

| | |
|---|---|
| MAGEA1 | Alpha chain DNA sequence |
| TCR 32-41 | ATGGTCCTGAAATTCTCCGTGTCCATTCTTTGGATTCAGTTGGCA |
| (also known | TGGGTGAGCACCCAGCTGCTGGAGCAGAGCCCTCAGTTTCTTAG |
| as clone | CATCCAAGAGGGAGAAAATCTCACTGTGTACTGCAACTCCTCAA |
| “TSC-204- | GTGTTTTCTCCAGCCTTCAATGGTACAGACAGGAGCCTGGGGA |
| C07”, “TSC- | AGGTCCTGTCCTCCTGGTGACAGTTGTTACTGGTGGAGAAGTG |
| 204-C7”, and | AAGAAGCTGAAGAGACTTACCTTTCAGTTTGGTGATGCAAGAAA |
| “TSC-204- | GGACAGTTCTCTCCACATCACTGCAGCCCAGCCTGGTGATACAG |
| C0702”) | GCCTCTACCTCTGTGCAGGAGATGAAAGTATTAGCTATGGAA |
| MGTM codon | AGCTGACATTTGGACAAGGGACCATCTTGACTGTCCATCCAaaca |
| optimized | tccagaaccccgaccccgccgtgtaccagctgagggactccaagtccagcgacaagagcgtgtgtctgtt |
| sequence | tacggacttcgacagccagaccaacgtgagtcaaagcaaggacagcgacgtctacataacggataagac |
| Note: This | cgtgctggacatgcggagcatggacttcaagagcaacagcgccgtggcctggtccaacaagagcgactt |
| clone was used | cgcctgcgccaacgccttcaacaacagcatcatccccgaggacaccttcttccccagcagcgacgtgccc |
| in FIG. 3 as | tgcgacgtgaaactggtggagaagtccttcgagacagacaccaatctgaactttcagaacctgctggtgat |
| MAGEA1 | cgtgctgcggattctgctgctgaaagtggccggcttcaatctgctgatgaccctgcggctgtggagc |
| TCR in | (SEQ ID NO: 11) |
| multiplex with | Alpha chain protein sequence |
| the HPV TCR | MVLKFSVSILWIQLAWVSTQLLEQSPQFLSIQEGENLTVYCNSSSVF |
| and in FIGS. | SSLQWYRQEPGEGPVLLVTVVTGGEVKKLKRLTFQFGDARKDSSL |
| 6 and 7-9 in | HITAAQPGDTGLYLCAGDESISYGKLTFGQGTILTVHPNiqnpdpavyq |
| multiplex with | lrdskssdksvclftdfdsqtnvsqskdsdvyitdktvldmrsmdfksn |
| other TCRs | savawsnksdfacanafnnsiipedtffpssdvpcdvklveksfetdtn |
| Alpha chain: | lnfqnllvivlrilllkvagfnllmtlrlws (SEQ ID NO: 12) |
| TRAV27/ | |
| TRAJ52/MGTM | |
| modified | |
| TRAC | |
| MAGEA1 | Beta chain DNA sequence |
| TCR 32-41 | ATGGGCTCCTGGACCCTCTGCTGTGTGTCCCTTTGCATCCTGGTT |
| (also known | GCAAAGCACACAGATGCTGGAGTTATCCAGTCACCCCGGCACGA |
| as clone | GGTGACAGAGATGGGACAAGAAGTGACTCTGAGATGTAAACCA |
| “TSC-204- | ATTTCAGGACATGACTACCTTTTCTGGTACAGACAGACCATGAT |
| C07”, “TSC- | GCGGGGACTGGAGTTGCTCATTTACTTCAACAACAACGTTCCT |
| 204-C7”, and | ATTGATGATTCAGGGATGCCCGAGGATCGCTTCTCAGCTAAGAT |
| “TSC-204- | GCCTAATGCATCATTCTCCACTCTGAAGATCCAGCCCTCAGAAC |
| C0702”) | CCAGGGACTCAGCTGTGTACTTCTGTGCCAGCAGTTTTCTCGG |
| MGTM codon | CTGGAATGAAAAACTGTTCTTTGGCAGTGGAACCCAGCTCTCT |
| optimized | GTCTTGgaagatctgaacaaggtgttccctccagaggtggccgtgttcgagccttctaaggccgagat |
| sequence | cgcccacacacaaaaagccaccctcgtgtgcctggccaccggctttttccccgaccacgtggaactgtctt |
| Note: This | ggtgggtcaacggcaaagaggtgcactccggcgtgtcaacggatccccagcctctgaaagaacagcctg |
| clone was used | ccctgaacgacagccggtactgcctgagctccagactgagagtgtccgccaccttctggcagaacccccg |
| in FIG. 3 as | gaaccacttcagatgccaggtgcagttttacggcctgagcgagaacgacgagtggacccaggacagagc |
| MAGEA1 | caagcccgtgacacaaatcgtgtctgccgaagcctggggaagagccgattgcggcatcaccagcgcctc |
| TCR in | ctatcaccagggcgtgctgagcgccacaatcctgtacgaaatcctgctgggcaaggccaccctgtacgcc |
| multiplex with | gtgctggtgtctgctctggtgctgatggccatggtcaagcggaaggacttt (SEQ ID NO: 13) |
| the HPV TCR | Beta chain protein sequence |
| and in FIGS. | MGSWTLCCVSLCILVAKHTDAGVIQSPRHEVTEMGQEVTLRCKPIS |
| 6 and 7-9 in | GHDYLFWYRQTMMRGLELLIYFNNNVPIDDSGMPEDRFSAKMPN |
| multiplex with | ASFSTLKIQPSEPRDSAVYFCASSFLGWNEKLFFGSGTQLSVLEdlnk |
| other TCRs | vfppevavfepskaeiahtqkatlvclatgffpdhvelswwvngkevh |
| Beta chain: | sgvstdpqplkeqpalndsryclssrlrvsatfwqnprnhfrcqvqfy |
| TRBV12-4/ | glsendewtqdrakpvtqivsaeawgradcgitsasyhqgvlsatily |
| TRBJ1-4/ | eillgkatlyavlvsalvlmamvkrkdf (SEQ ID NO: 14) |
| MGTM | |
| modified | |
| TRBC1 | |
| Complete Beta | ATGGGCTCCTGGACCCTCTGCTGTGTGTCCCTTTGCATCCTGGTT |
| and Alpha | GCAAAGCACACAGATGCTGGAGTTATCCAGTCACCCCGGCACGA |
| ORF DNA | GGTGACAGAGATGGGACAAGAAGTGACTCTGAGATGTAAACCA |
| Sequence | ATTTCAGGACATGACTACCTTTTCTGGTACAGACAGACCATGAT |
| | GCGGGGACTGGAGTTGCTCATTTACTTCAACAACAACGTTCCT |
| | ATTGATGATTCAGGGATGCCCGAGGATCGCTTCTCAGCTAAGAT |
| | GCCTAATGCATCATTCTCCACTCTGAAGATCCAGCCCTCAGAAC |
| | CCAGGGACTCAGCTGTGTACTTCTGTGCCAGCAGTTTTCTCGG |
| | CTGGAATGAAAAACTGTTCTTTGGCAGTGGAACCCAGCTCTCT |
| | GTCTTGgaagatctgaacaaggtgttccctccagaggtggccgtgttcgagccttctaaggccgagat |
| | cgcccacacacaaaaagccaccctcgtgtgcctggccaccggctttttccccgaccacgtggaactgtctt |
| | ggtgggtcaacggcaaagaggtgcactccggcgtgtcaacggatccccagcctctgaaagaacagcctg |
| | ccctgaacgacagccggtactgcctgagctccagactgagagtgtccgccaccttctggcagaacccccg |
| | gaaccacttcagatgccaggtgcagttttacggcctgagcgagaacgacgagtggacccaggacagagc |
| | caagcccgtgacacaaatcgtgtctgccgaagcctggggaagagccgattgcggcatcaccagcgcctc |
| | ctatcaccagggcgtgctgagcgccacaatcctgtacgaaatcctgctgggcaaggccaccctgtacgcc |
| | gtgctggtgtctgctctggtgctgatggccatggtcaagcggaaggactttGGCAGCGGCAGAG |
| | CCAAAAGGTCCGGGAGCGGTGCGACAAACTTTAGCCTGTTGAAACAA |
| | GCCGGCGACGTTGAAGAGAACCCCGGACCTATGGTCCTGAAATTC |
| | TCCGTGTCCATTCTTTGGATTCAGTTGGCATGGGTGAGCACCCAG |
| | CTGCTGGAGCAGAGCCCTCAGTTTCTTAGCATCCAAGAGGGAGA |
| | AAATCTCACTGTGTACTGCAACTCCTCAAGTGTTTTCTCCAGCC |
| | TTCAATGGTACAGACAGGAGCCTGGGGAAGGTCCTGTCCTCCTG |
| | GTGACAGTTGTTACTGGTGGAGAAGTGAAGAAGCTGAAGAGA |
| | CTTACCTTTCAGTTTGGTGATGCAAGAAAGGACAGTTCTCTCCAC |
| | ATCACTGCAGCCCAGCCTGGTGATACAGGCCTCTACCTCTGTGC |
| | AGGAGATGAAAGTATTAGCTATGGAAAGCTGACATTTGGACA |
| | AGGGACCATCTTGACTGTCCATCCAaacatccagaaccccgaccccgccgtgtac |
| | cagctgagggactccaagtccagcgacaagagcgtgtgtctgtttacggacttcgacagccagaccaacg |
| | tgagtcaaagcaaggacagcgacgtctacataacggataagaccgtgctggacatgcggagcatggactt |
| | caagagcaacagcgccgtggcctggtccaacaagagcgacttcgcctgcgccaacgccttcaacaacag |
| | catcatccccgaggacaccttcttccccagcagcgacgtgccctgcgacgtgaaactggtggagaagtcc |
| | ttcgagacagacaccaatctgaactttcagaacctgctggtgatcgtgctgcggattctgctgctgaaagtg |
| | gccggcttcaatctgctgatgaccctgcggctgtggagc (SEQ ID NO: 15) |
| Complete Beta | MGSWTLCCVSLCILVAKHTDAGVIQSPRHEVTEMGQEVTLRCKPIS |
| and Alpha | GHDYLFWYRQTMMRGLELLIYFNNNVPIDDSGMPEDRFSAKMPN |
| ORF Protein | ASFSTLKIQPSEPRDSAVYFCASSFLGWNEKLFFGSGTQLSVLEdlnk |
| Sequence | vfppevavfepskaeiahtqkatlvclatgffpdhvelswwvngkevhsgvstdpqplkeqpalndsr |
| | yclssrlrvsatfwqnprnhfrcqvqfyglsendewtqdrakpvtqivsaeawgradcgitsasyhqgv |
| | lsatilyeillgkatlyavlvsalvlmamvkrkdfGSGRAKRSGSGATNFSLLKQAGDVE |
| | ENPGPMVLKFSVSILWIQLAWVSTQLLEQSPQFLSIQEGENLTVYCN |
| | SSSVFSSLQWYRQEPGEGPVLLVTVVTGGEVKKLKRLTFQFGDAR |
| | KDSSLHITAAQPGDTGLYLCAGDESISYGKLTFGQGTILTVHPNiqn |
| | pdpavyqlrdskssdksvclftdfdsqtnvsqskdsdvyitdktvldmrsmdfksnsavawsnksdfa |
| | canafnnsiipedtffpssdvpcdvklveksfetdtnlnfqnllvivlrilllkvagfnllmtlrlws (SEQ |
| | ID NO: 16) |
| MAGEC2 | Alpha chain DNA sequence |
| TCR 8-3 wild | ATGGAGAAGAATCCTTTGGCAGCCCCACTTCTTATCCTCTGGTTT |
| type sequence | CATCTTGACTGCGTGAGCAGCATTCTGAACGTGGAACAAAGTCC |
| Alpha chain: | TCAGTCACTGCATGTTCAGGAGGGAGACAGCACCAATTTCACCT |
| TRAV24*01F/ | GCAGCTTCCCTTCCAGCAATTTTTATGCCCTTCACTGGTACAGA |
| TRAJ32*02/ | TGGGAAACTGCAAAAAGCCCCGAGGCCTTGTTTGTTATGACTCT |
| TRAC | TAATGGGGATGAAAAGAAGAAAGGACGCATTAGTGCCACTCTT |
| | AATACCAAGGAGGGTTACAGCTATTTGTATATCAAAGGATCCCA |
| | GCCTGAGGACTCAGCCACATACCTCTGTGCCTCCGGAAGTGGT |
| | GGTGCTACAAACAAGCTCATCTTTGGAACTGGCACTCTGCTTG |
| | CTGTCCAGCCAAatatccagaaccctgaccctgccgtgtaccagctgagagactctaaatccag |
| | tgacaagtctgtctgcctattcaccgattttgattctcaaacaaatgtgtcacaaagtaaggattctgatgtgtat |
| | atcacagacaaaactgtgctagacatgaggtctatggacttcaagagcaacagtgctgtggcctggagcaa |
| | caaatctgactttgcatgtgcaaacgccttcaacaacagcattattccagaagacaccttcttccccagccca |
| | gaaagttcctgtgatgtcaagctggtcgagaaaagctttgaaacagatacgaacctaaactttcaaaacctgt |
| | cagtgattgggttccgaatcctcctcctgaaagtggccgggtttaatctgctcatgacgctgcggctgtggtc |
| | cagc (SEQ ID NO: 17) |
| | alpha chain protein sequence |
| | MEKNPLAAPLLILWFHLDCVSSILNVEQSPQSLHVQEGDSTNFTCSF |
| | PSSNFYALHWYRWETAKSPEALFVMTLNGDEKKKGRISATLNTKE |
| | GYSYLYIKGSQPEDSATYLCASGSGGATNKLIFGTGTLLAVQPniqn |
| | pdpavyqlrdskssdksvclftdfdsqtnvsqskdsdvyitdktvldmrsmdfksnsavawsnksdfa |
| | canafnnsiipedtffpspesscdvklveksfetdtnlnfqnlsvigfrilllkvagfnllmtlrlwss |
| | (SEQ ID NO: 18) |
| MAGEC2 | Beta chain DNA sequence |
| TCR 8-3 wild | ATGAGCCCAATTTTCACCTGCATCACAATCCTTTGTCTGCTGGCT |
| type sequence | GCAGGTTCTCCTGGTGAAGAAGTCGCCCAGACTCCAAAACATCT |
| Beta chain: | TGTCAGAGGGGAAGGACAGAAAGCAAAACTTTATTGTGCCCCA |
| TRBV16*01/ | ATTAAAGGACACAGTTATGTTTTCTGGTACCAACAGGTCCTGAA |
| TRBJ1-1*01/ | AAACGAGTTCAAGTTCTTGATTTCCTTCCAGAATGAAAATGTCT |
| TRBC1 | TTGATGAAACAGGTATGCCCAAGGAAAGATTTTCAGCTAAGTGC |
| | CTCCCAAATTCACCCTGTAGCCTTGAGATCCAGGCTACTAAGCTT |
| | GAGGATTCAGCAGTGTATTTTTGTGCCAGCAGCCAATCACGGA |
| | GCCTTAGGGGCACTGAAGCTTTCTTTGGACAAGGCACCAGAC |
| | TCACAGTTGTTGaggacctgaacaaggtgttcccacccgaggtcgctgtgtttgagccatcaga |
| | agcagagatctcccacacccaaaaggccacactggtgtgcctggccacaggcttcttccctgaccacgtg |
| | gagctgagctggtgggtgaatgggaaggaggtgcacagtggggtcagcacggacccgcagcccctcaa |
| | ggagcagcccgccctcaatgactccagatactgcctgagcagccgcctgagggtctcggccaccttctgg |
| | cagaacccccgcaaccacttccgctgtcaagtccagttctacgggctctcggagaatgacgagtggaccc |
| | aggatagggccaaacccgtcacccagatcgtcagcgccgaggcctggggtagagcagactgtggcttta |
| | cctcggtgtcctaccagcaaggggtcctgtctgccaccatcctctatgagatcctgctagggaaggccacc |
| | ctgtatgctgtgctggtcagcgcccttgtgttgatggccatggtcaagagaaaggatttc (SEQ ID |
| | NO: 19) |
| | Beta chain protein sequence |
| | MSPIFTCITILCLLAAGSPGEEVAQTPKHLVRGEGQKAKLYCAPIKG |
| | HSYVFWYQQVLKNEFKFLISFQNENVFDETGMPKERFSAKCLPNSP |
| | CSLEIQATKLEDSAVYFCASSQSRSLRGTEAFFGQGTRLTVVEdlnkv |
| | fppevavfepseaeishtqkatlvclatgffpdhvelswwvngkevhsgvstdpqplkeqpalndsry |
| | clssrlrvsatfwqnprnhfrcqvqfyglsendewtqdrakpvtqivsaeawgradcgftsvsyqqgvl |
| | satilyeillgkatlyavlvsalvlmamvkrkdf (SEQ ID NO: 20) |
| MAGEC2 | Alpha chain DNA sequence |
| TCR 8-3 HM | ATGGAGAAGAATCCTTTGGCAGCCCCACTTCTTATCCTCTGGTTT |
| codon | CATCTTGACTGCGTGAGCAGCATTCTGAACGTGGAACAAAGTCC |
| optimized | TCAGTCACTGCATGTTCAGGAGGGAGACAGCACCAATTTCACCT |
| sequence | GCAGCTTCCCTTCCAGCAATTTTTATGCCCTTCACTGGTACAGA |
| Note: This | TGGGAAACTGCAAAAAGCCCCGAGGCCTTGTTTGTTATGACTCT |
| clone was used | TAATGGGGATGAAAAGAAGAAAGGACGCATTAGTGCCACTCTT |
| in FIG. 4 as | AATACCAAGGAGGGTTACAGCTATTTGTATATCAAAGGATCCCA |
| the MAGEC2 | GCCTGAGGACTCAGCCACATACCTCTGTGCCTCCGGAAGTGGT |
| TCR in | GGTGCTACAAACAAGCTCATCTTTGGAACTGGCACTCTGCTTG |
| multiplex with | CTGTCCAGCCAAacattcaaaacccagaacccgccgtctaccagctgaaagacccgaggtctc |
| the MAGEA1 | aagactctacgttgtgcttgttcaccgatttcgacagtcagataaatgtgcctaagaccatggagagtggcac |
| TCR. Also, a | tttcatcactgacaaatgtgtgttggacatgaaggctatggacagcaagtcaaacggcgcgattgcttggtc |
| TCR-alone | caaccaaacttctttcacgtgccaggacatcttcaaggagacaaacgccacctatccatcctctgatgttccg |
| vector was | tgcgatgcgactcttaccgagaaaagcttcgagacggacatgaacttgaacttccaaaacctgcttgtgatg |
| used for this | gtactgcgaatacttcttcttaaggtggcgggcttcaatttgctcatgacactcagactttggtctagc (SEQ |
| experiment | ID NO: 21) |
| such that no | Alpha chain protein sequence |
| additional | MEKNPLAAPLLILWFHLDCVSSILNVEQSPQSLHVQEGDSTNFTCSF |
| elements (e.g., | PSSNFYALHWYRWETAKSPEALFVMTLNGDEKKKGRISATLNTKE |
| CD8alpha/beta | GYSYLYIKGSQPEDSATYLCASGSGGATNKLIFGTGTLLAVQPNiqn |
| co-receptor, | pepavyqlkdprsqdstlclftdfdsqinvpktmesgtfitdkcvldmkamdsksngaiawsnqtsftc |
| DN-TGFBRII, | qdifketnatypssdvpcdatlteksfetdmnlnfqnllvmvlrilllkvagfnllmtlrlwss |
| and the like | (SEQ ID NO: 22) |
| were not | |
| expressed). | |
| Alpha chain: | |
| TRAV24*01F/ | |
| TRAJ32*02/ | |
| codon- | |
| optimized | |
| mouse TRAC | |
| MAGEC2 | Beta chain DNA sequence |
| TCR 8-3 HM | ATGAGCCCAATTTTCACCTGCATCACAATCCTTTGTCTGCTGGCT |
| codon | GCAGGTTCTCCTGGTGAAGAAGTCGCCCAGACTCCAAAACATCT |
| optimized | TGTCAGAGGGGAAGGACAGAAAGCAAAACTTTATTGTGCCCCA |
| sequence | ATTAAAGGACACAGTTATGTTTTCTGGTACCAACAGGTCCTGAA |
| Note: This | AAACGAGTTCAAGTTCTTGATTTCCTTCCAGAATGAAAATGTCT |
| clone was used | TTGATGAAACAGGTATGCCCAAGGAAAGATTTTCAGCTAAGTGC |
| in FIG. 4 as | CTCCCAAATTCACCCTGTAGCCTTGAGATCCAGGCTACTAAGCTT |
| the MAGEC2 | GAGGATTCAGCAGTGTATTTTTGTGCCAGCAGCCAATCACGGA |
| TCR in | GCCTTAGGGGCACTGAAGCTTTCTTTGGACAAGGCACCAGAC |
| multiplex with | TCACAGTTGTTGaagatcttcgaaacgtaacccctccaaaagtgagtctctttgaaccgagtaag |
| the MAGEA1 | gctgagatcgcgaacaaacaaaaggcgaccctcgtctgtcttgcgcgaggattttttcccgaccacgtgga |
| TCR. Also, a | gttgtcttggtgggtaaacggtaaggaagtacacagcggtgtttgcaccgaccctcaagcctacaaggaat |
| TCR-alone | ctaactattcatactgcctttcatcccgacttagggtttctgctaccttttggcacaatccgaggaatcactttag |
| vector was | gtgtcaagtacagttccacggattgtcagaggaggataaatggccggagggctccccgaagccggttacg |
| used for this | cagaacattagtgcggaagcctggggacgagcagactgcggtatcacgtctgccagctatcagcaaggc |
| experiment | gttctgtcagcgacaattctgtacgaaatacttttgggtaaggctacattgtatgcggtattggtgtctacgctg |
| such that no | gtagtcatggccatggtgaaacgaaaaaactca (SEQ ID NO: 23) |
| additional | Beta chain protein sequence |
| elements (e.g., | MSPIFTCITILCLLAAGSPGEEVAQTPKHLVRGEGQKAKLYCAPIKG |
| CD8alpha/beta | HSYVFWYQQVLKNEFKFLISFQNENVFDETGMPKERFSAKCLPNSP |
| co-receptor, | CSLEIQATKLEDSAVYFCASSQSRSLRGTEAFFGQGTRLTVVEdlrnv |
| DN-TGFBRII, | tppkvslfepskaeiankqkatlvclargffpdhvelswwvngkevhsgvctdpqaykesnysyclss |
| and the like | rlrvsatfwhnprnhfrcqvqfhglseedkwpegspkpvtqnisaeawgradcgitsasyqqgvlsati |
| were not | lyeillgkatlyavlvstlvvmamvkrkns (SEQ ID NO: 24) |
| expressed). | |
| Beta chain: | |
| TRBV16*01/ | |
| TRBJ1-1*01/ | |
| codon- | |
| optimized | |
| mouse TRBC | |
| Complete Beta | ATGAGCCCAATTTTCACCTGCATCACAATCCTTTGTCTGCTGGCT |
| and Alpha | GCAGGTTCTCCTGGTGAAGAAGTCGCCCAGACTCCAAAACATCT |
| ORF DNA | TGTCAGAGGGGAAGGACAGAAAGCAAAACTTTATTGTGCCCCA |
| Sequence (The | ATTAAAGGACACAGTTATGTTTTCTGGTACCAACAGGTCCTGAA |
| underlined | AAACGAGTTCAAGTTCTTGATTTCCTTCCAGAATGAAAATGTCT |
| italic region in | TTGATGAAACAGGTATGCCCAAGGAAAGATTTTCAGCTAAGTGC |
| the “Furin- | CTCCCAAATTCACCCTGTAGCCTTGAGATCCAGGCTACTAAGCTT |
| P2A” site | GAGGATTCAGCAGTGTATTTTTGTGCCAGCAGCCAATCACGGA |
| encodes a | GCCTTAGGGGCACTGAAGCTTTCTTTGGACAAGGCACCAGAC |
| sequence | TCACAGTTGTTGaagatcttcgaaacgtaacccctccaaaagtgagtctctttgaaccgagtaag |
| allowing for | gctgagatcgcgaacaaacaaaaggcgaccctcgtctgtcttgcgcgaggattttttcccgaccacgtgga |
| expression of | gttgtcttggtgggtaaacggtaaggaagtacacagcggtgtttgcaccgaccctcaagcctacaaggaat |
| two | ctaactattcatactgcctttcatcccgacttagggtttctgctaccttttggcacaatccgaggaatcactttag |
| polypeptide | gtgtcaagtacagttccacggattgtcagaggaggataaatggccggagggctccccgaagccggttacg |
| chains in a | cagaacattagtgcggaagcctggggacgagcagactgcggtatcacgtctgccagctatcagcaaggc |
| single | gttctgtcagcgacaattctgtacgaaatacttttgggtaaggctacattgtatgcggtattggtgtctacgctg |
| cassette”) | gtagtcatggccatggtgaaacgaaaaaactcaagagccaaaagaagcgggagcggtgcgacaaact |
| | ttagcctgttgaaacaagccggcgacgttgaagagaaccccggacctATGGAGAAGAATC |
| | CTTTGGCAGCCCCACTTCTTATCCTCTGGTTTCATCTTGACTGCG |
| | TGAGCAGCATTCTGAACGTGGAACAAAGTCCTCAGTCACTGCAT |
| | GTTCAGGAGGGAGACAGCACCAATTTCACCTGCAGCTTCCCTTC |
| | CAGCAATTTTTATGCCCTTCACTGGTACAGATGGGAAACTGCA |
| | AAAAGCCCCGAGGCCTTGTTTGTTATGACTCTTAATGGGGATG |
| | AAAAGAAGAAAGGACGCATTAGTGCCACTCTTAATACCAAGGA |
| | GGGTTACAGCTATTTGTATATCAAAGGATCCCAGCCTGAGGACT |
| | CAGCCACATACCTCTGTGCCTCCGGAAGTGGTGGTGCTACAA |
| | ACAAGCTCATCTTTGGAACTGGCACTCTGCTTGCTGTCCAGCCA |
| | Aacattcaaaacccagaacccgccgtctaccagctgaaagacccgaggtctcaagactctacgttgtgctt |
| | gttcaccgatttcgacagtcagataaatgtgcctaagaccatggagagtggcactttcatcactgacaaatgt |
| | gtgttggacatgaaggctatggacagcaagtcaaacggcgcgattgcttggtccaaccaaacttctttcacg |
| | tgccaggacatcttcaaggagacaaacgccacctatccatcctctgatgttccgtgcgatgcgactcttacc |
| | gagaaaagcttcgagacggacatgaacttgaacttccaaaacctgcttgtgatggtactgcgaatacttcttc |
| | ttaaggtggcgggcttcaatttgctcatgacactcagactttggtctagc (SEQ ID NO: 25) |
| Complete Beta | MSPIFTCITILCLLAAGSPGEEVAQTPKHLVRGEGQKAKLYCAPIKG |
| and Alpha | HSYVFWYQQVLKNEFKFLISFQNENVFDETGMPKERFSAKCLPNSP |
| ORF Protein | CSLEIQATKLEDSAVYFCASSQSRSLRGTEAFFGQGTRLTVVEdlrnv |
| Sequence (The | tppkvslfepskaeiankqkatlvclargffpdhvelswwvngkevhsgvctdpqaykesnysyclss |
| underlined | rlrvsatfwhnprnhfrcqvqfhglseedkwpegspkpvtqnisaeawgradcgitsasyqqgvlsati |
| italic region in | lyeillgkatlyavlvstlvvmamvkrknsrakrsgsgatnfsllkqagdveenpgpMEKNPLAA |
| the “Furin- | PLLILWFHLDCVSSILNVEQSPQSLHVQEGDSTNFTCSFPSSNFYALH |
| P2A” site | WYRWETAKSPEALFVMTLNGDEKKKGRISATLNTKEGYSYLYIK |
| allows | GSQPEDSATYLCASGSGGATNKLIFGTGTLLAVQPNiqnpepavyqlkdp |
| expression of | rsqdstlclftdfdsqinvpktmesgtfitdkcvldmkamdsksngaiawsnqtsftcqdifketnatyp |
| two | ssdvpcdatlteksfetdmnlnfqnllvmvlrilllkvagfnllmtlrlwss (SEQ ID NO: 26) |
| polypeptide | |
| chains in a | |
| single | |
| cassette”)”) | |
| MAGEC2 | Alpha chain DNA sequence |
| TCR 8-3 (also | ATGGAGAAGAATCCTTTGGCAGCCCCACTTCTTATCCTCTGGTTT |
| known as | CATCTTGACTGCGTGAGCAGCATTCTGAACGTGGAACAAAGTCC |
| clone “TCR | TCAGTCACTGCATGTTCAGGAGGGAGACAGCACCAATTTCACCT |
| LD8-3”) | GCAGCTTCCCTTCCAGCAATTTTTATGCCCTTCACTGGTACAGA |
| MGTM codon | TGGGAAACTGCAAAAAGCCCCGAGGCCTTGTTTGTTATGACTCT |
| optimized | TAATGGGGATGAAAAGAAGAAAGGACGCATTAGTGCCACTCTT |
| sequence | AATACCAAGGAGGGTTACAGCTATTTGTATATCAAAGGATCCCA |
| Alpha chain: | GCCTGAGGACTCAGCCACATACCTCTGTGCCTCCGGAAGTGGT |
| TRAV24*01F/ | GGTGCTACAAACAAGCTCATCTTTGGAACTGGCACTCTGCTTG |
| TRAJ32*02/ | CTGTCCAGCCAAacatccagaaccccgaccccgccgtgtaccagctgagggactccaagtcc |
| MGTM | agcgacaagagcgtgtgtctgtttacggacttcgacagccagaccaacgtgagtcaaagcaaggacagc |
| modified | gacgtctacataacggataagaccgtgctggacatgcggagcatggacttcaagagcaacagcgccgtg |
| TRAC | gcctggtccaacaagagcgacttcgcctgcgccaacgccttcaacaacagcatcatccccgaggacacct |
| | tcttccccagcagcgacgtgccctgcgacgtgaaactggtggagaagtccttcgagacagacaccaatct |
| | gaactttcagaacctgctggtgatcgtgctgcggattctgctgctgaaagtggccggcttcaatctgctgatg |
| | accctgcggctgtggagcagc (SEQ ID NO: 27) |
| | Alpha chain protein sequence |
| | MEKNPLAAPLLILWFHLDCVSSILNVEQSPQSLHVQEGDSTNFTCSF |
| | PSSNFYALHWYRWETAKSPEALFVMTLNGDEKKKGRISATLNTKE |
| | GYSYLYIKGSQPEDSATYLCASGSGGATNKLIFGTGTLLAVQPNiqn |
| | pdpavyqlrdskssdksvclftdfdsqtnvsqskdsdvyitdktvldmrsmdfksnsavawsnksdfa |
| | canafnnsiipedtffpssdvpcdvklveksfetdtnlnfqnllvivlrilllkvagfnllmtlrlwss |
| | (SEQ ID NO: 28) |
| MAGEC2 | Beta chain DNA sequence |
| TCR 8-3 (also | ATGAGCCCAATTTTCACCTGCATCACAATCCTTTGTCTGCTGGCT |
| known as | GCAGGTTCTCCTGGTGAAGAAGTCGCCCAGACTCCAAAACATCT |
| clone “TCR | TGTCAGAGGGGAAGGACAGAAAGCAAAACTTTATTGTGCCCCA |
| LD8-3”) | ATTAAAGGACACAGTTATGTTTTCTGGTACCAACAGGTCCTGAA |
| MGTM codon | AAACGAGTTCAAGTTCTTGATTTCCTTCCAGAATGAAAATGTCT |
| optimized | TTGATGAAACAGGTATGCCCAAGGAAAGATTTTCAGCTAAGTGC |
| sequence | CTCCCAAATTCACCCTGTAGCCTTGAGATCCAGGCTACTAAGCTT |
| Beta chain: | GAGGATTCAGCAGTGTATTTTTGTGCCAGCAGCCAATCACGGA |
| TRBV16*01/ | GCCTTAGGGGCACTGAAGCTTTCTTTGGACAAGGCACCAGAC |
| TRBJ1-1*01/ | TCACAGTTGTTGaagatctgaacaaggtgttccctccagaggtggccgtgttcgagccttctaag |
| MGTM | gccgagatcgcccacacacaaaaagccaccctcgtgtgcctggccaccggctttttccccgaccacgtgg |
| modified | aactgtcttggtgggtcaacggcaaagaggtgcactccggcgtgtcaacggatccccagcctctgaaaga |
| TRBC | acagcctgccctgaacgacagccggtactgcctgagctccagactgagagtgtccgccaccttctggcag |
| | aacccccggaaccacttcagatgccaggtgcagttttacggcctgagcgagaacgacgagtggacccag |
| | gacagagccaagcccgtgacacaaatcgtgtctgccgaagcctggggaagagccgattgcggcatcacc |
| | agcgcctcctatcaccagggcgtgctgagcgccacaatcctgtacgaaatcctgctgggcaaggccaccc |
| | tgtacgccgtgctggtgtctgctctggtgctgatggccatggtcaagcggaaggacttt (SEQ ID NO: |
| | 29) |
| | Beta chain protein sequence |
| | MSPIFTCITILCLLAAGSPGEEVAQTPKHLVRGEGQKAKLYCAPIKG |
| | HSYVFWYQQVLKNEFKFLISFQNENVFDETGMPKERFSAKCLPNSP |
| | CSLEIQATKLEDSAVYFCASSQSRSLRGTEAFFGQGTRLTVVEdlnkv |
| | fppevavfepskaeiahtqkatlvclatgffpdhvelswwvngkevhsgvstdpqplkeqpalndsry |
| | clssrlrvsatfwqnprnhfrcqvqfyglsendewtqdrakpvtqivsaeawgradcgitsasyhqgvl |
| | satilyeillgkatlyavlvsalvlmamvkrkdf (SEQ ID NO: 30) |
| Complete Beta | ATGAGCCCAATTTTCACCTGCATCACAATCCTTTGTCTGCTGGCT |
| and Alpha | GCAGGTTCTCCTGGTGAAGAAGTCGCCCAGACTCCAAAACATCT |
| ORF DNA | TGTCAGAGGGGAAGGACAGAAAGCAAAACTTTATTGTGCCCCA |
| Sequence | ATTAAAGGACACAGTTATGTTTTCTGGTACCAACAGGTCCTGAA |
| | AAACGAGTTCAAGTTCTTGATTTCCTTCCAGAATGAAAATGTCT |
| | TTGATGAAACAGGTATGCCCAAGGAAAGATTTTCAGCTAAGTGC |
| | CTCCCAAATTCACCCTGTAGCCTTGAGATCCAGGCTACTAAGCTT |
| | GAGGATTCAGCAGTGTATTTTTGTGCCAGCAGCCAATCACGGA |
| | GCCTTAGGGGCACTGAAGCTTTCTTTGGACAAGGCACCAGAC |
| | TCACAGTTGTTGaagatctgaacaaggtgttccctccagaggtggccgtgttcgagccttctaag |
| | gccgagatcgcccacacacaaaaagccaccctcgtgtgcctggccaccggctttttccccgaccacgtgg |
| | aactgtcttggtgggtcaacggcaaagaggtgcactccggcgtgtcaacggatccccagcctctgaaaga |
| | acagcctgccctgaacgacagccggtactgcctgagctccagactgagagtgtccgccaccttctggcag |
| | aacccccggaaccacttcagatgccaggtgcagttttacggcctgagcgagaacgacgagtggacccag |
| | gacagagccaagcccgtgacacaaatcgtgtctgccgaagcctggggaagagccgattgcggcatcacc |
| | agcgcctcctatcaccagggcgtgctgagcgccacaatcctgtacgaaatcctgctgggcaaggccaccc |
| | tgtacgccgtgctggtgtctgctctggtgctgatggccatggtcaagcggaaggactttggcagcggcaga |
| | gccaaaagaagcgggagcggtgcgacaaactttagcctgttgaaacaagccggcgacgttgaagag |
| | aaccccggacctATGGAGAAGAATCCTTTGGCAGCCCCACTTCTTATC |
| | CTCTGGTTTCATCTTGACTGCGTGAGCAGCATTCTGAACGTGGA |
| | ACAAAGTCCTCAGTCACTGCATGTTCAGGAGGGAGACAGCACCA |
| | ATTTCACCTGCAGCTTCCCTTCCAGCAATTTTTATGCCCTTCAC |
| | TGGTACAGATGGGAAACTGCAAAAAGCCCCGAGGCCTTGTTTGT |
| | TATGACTCTTAATGGGGATGAAAAGAAGAAAGGACGCATTAG |
| | TGCCACTCTTAATACCAAGGAGGGTTACAGCTATTTGTATATCA |
| | AAGGATCCCAGCCTGAGGACTCAGCCACATACCTCTGTGCCTCC |
| | GGAAGTGGTGGTGCTACAAACAAGCTCATCTTTGGAACTGGC |
| | ACTCTGCTTGCTGTCCAGCCAAacatccagaaccccgaccccgccgtgtaccagctg |
| | agggactccaagtccagcgacaagagcgtgtgtctgtttacggacttcgacagccagaccaacgtgagtc |
| | aaagcaaggacagcgacgtctacataacggataagaccgtgctggacatgcggagcatggacttcaaga |
| | gcaacagcgccgtggcctggtccaacaagagcgacttcgcctgcgccaacgccttcaacaacagcatcat |
| | ccccgaggacaccttcttccccagcagcgacgtgccctgcgacgtgaaactggtggagaagtccttcgag |
| | acagacaccaatctgaactttcagaacctgctggtgatcgtgctgcggattctgctgctgaaagtggccggc |
| | ttcaatctgctgatgaccctgcggctgtggagcagc (SEQ ID NO: 31) |
| Complete Beta | MSPIFTCITILCLLAAGSPGEEVAQTPKHLVRGEGQKAKLYCAPIKG |
| and Alpha | HSYVFWYQQVLKNEFKFLISFQNENVFDETGMPKERFSAKCLPNSP |
| ORF Protein | CSLEIQATKLEDSAVYFCASSQSRSLRGTEAFFGQGTRLTVVEdlnkv |
| Sequence | fppevavfepskaeiahtqkatlvclatgffpdhvelswwvngkevhsgvstdpqplkeqpalndsry |
| | clssrlrvsatfwqnprnhfrcqvqfyglsendewtqdrakpvtqivsaeawgradcgitsasyhqgvl |
| | satilyeillgkatlyavlvsalvlmamvkrkdfgsgrakrsgsgatnfsllkqagdveenpgpMEKN |
| | PLAAPLLILWFHLDCVSSILNVEQSPQSLHVQEGDSTNFTCSFPSSNF |
| | YALHWYRWETAKSPEALFVMTLNGDEKKKGRISATLNTKEGYSY |
| | LYIKGSQPEDSATYLCASGSGGATNKLIFGTGTLLAVQPNiqnpdpav |
| | yqlrdskssdksvclftdfdsqtnvsqskdsdvyitdktvldmrsmdfksnsavawsnksdfacanaf |
| | nnsiipedtffpssdvpcdvklveksfetdtnlnfqnllvivlrilllkvagfnllmtlrlwss (SEQ ID |
| | NO: 32) |
| E7-11-28 | Alpha chain DNA sequence |
| MGTM codon | ATGCTGCTGATCACCTCCATGCTGGTGCTGTGGATGCAGCTGAG |
| optimized | CCAAGTGAACGGCCAGCAAGTGATGCAGATCCCTCAGTACCAGC |
| sequence (also | ACGTGCAAGAAGGCGAGGACTTCACCACCTACTGCAACAGCAG |
| known as | CACCACACTGAGCAACATCCAGTGGTACAAGCAGCGGCCTGGC |
| “TCR28” or | GGACACCCTGTGTTTCTGATCCAGCTGGTCAAGTCCGGCGAAG |
| “28”) | TGAAGAAGCAGAAGCGGCTGACCTTCCAGTTCGGCGAGGCCAA |
| Alpha chain: | GAAGAACAGCAGCCTGCACATCACCGCCACACAGACCACAGAT |
| TRAV25/ | GTGGGCACCTACTTCTGCGCTGGCATCGGTAGCAGCAACACC |
| TRAJ37/MGTM | GGTAAGCTCATCTTTGGGCAAGGGACAACTTTACAAGTAAAAC |
| modified | CAGacatccagaaccccgaccccgccgtgtaccagctgagggactccaagtccagcgacaagagcgt |
| TRAC | gtgtctgtttacggacttcgacagccagaccaacgtgagtcaaagcaaggacagcgacgtctacataacg |
| | gataagaccgtgctggacatgcggagcatggacttcaagagcaacagcgccgtggcctggtccaacaag |
| | agcgacttcgcctgcgccaacgccttcaacaacagcatcatccccgaggacaccttcttccccagcagcg |
| | acgtgccctgcgacgtgaaactggtggagaagtccttcgagacagacaccaatctgaactttcagaacctg |
| | ctggtgatcgtgctgcggattctgctgctgaaagtggccggcttcaatctgctgatgaccctgcggctgtgg |
| | agc (SEQ ID NO: 33) |
| | Alpha chain protein sequence |
| | MLLITSMLVLWMQLSQVNGQQVMQIPQYQHVQEGEDFTTYCNSS |
| | TTLSNIQWYKQRPGGHPVFLIQLVKSGEVKKQKRLTFQFGEAKKN |
| | SSLHITATQTTDVGTYFCAGIGSSNTGKLIFGQGTTLQVKPDiqnpdp |
| | avyqlrdskssdksvclftdfdsqtnvsqskdsdvyitdktvldmrsmdfksnsavawsnksdfacan |
| | afnnsiipedtffpssdvpcdvklveksfetdtnlnfqnllvivlrilllkvagfnllmtlrlws (SEQ ID |
| | NO: 34) |
| E7-11-28 | Beta chain DNA sequence |
| MGTM codon | ATGGATACCTGGCTCGTGTGTTGGGCCATCTTTAGCCTGCTGAA |
| optimized | GGCCGGACTGACCGAGCCTGAAGTGACCCAGACTCCAAGCCATC |
| sequence | AAGTGACTCAGATGGGGCAAGAAGTCATTCTGCGTTGCGTGCCC |
| Beta chain: | ATCAGCAACCACCTGTACTTTTATTGGTATCGCCAGATCCTGGG |
| TRBV2/ | CCAGAAAGTGGAATTCCTGGTGTCCTTCTACAACAATGAGATC |
| TRBJ2-7/MGTM | TCCGAGAAGTCCGAGATCTTCGACGACCAGTTCTCCGTGGAAAG |
| modified | ACCCGACGGCAGCAACTTCACACTGAAGATCCGGTCTACCAAAC |
| TRBC | TTGAGGACTCCGCTATGTATTTTTGTGCAATCACAGGTCGCGT |
| | TTCATATGAGCAATATTTCGGGCCGGGCACCAGGCTCACGGTC |
| | ACAgaagatctgaacaaggtgttccctccagaggtggccgtgttcgagccttctaaggccgagatcgcc |
| | cacacacaaaaagccaccctcgtgtgcctggccaccggctttttccccgaccacgtggaactgtcttggtg |
| | ggtcaacggcaaagaggtgcactccggcgtgtcaacggatccccagcctctgaaagaacagcctgccct |
| | gaacgacagccggtactgcctgagctccagactgagagtgtccgccaccttctggcagaacccccggaa |
| | ccacttcagatgccaggtgcagttttacggcctgagcgagaacgacgagtggacccaggacagagccaa |
| | gcccgtgacacaaatcgtgtctgccgaagcctggggaagagccgattgcggcatcaccagcgcctcctat |
| | caccagggcgtgctgagcgccacaatcctgtacgaaatcctgctgggcaaggccaccctgtacgccgtg |
| | ctggtgtctgctctggtgctgatggccatggtcaagcggaaggactttggcagcggcagagccaaaaggt |
| | ccgggagcggt (SEQ ID NO: 35) |
| | Beta chain protein sequence |
| | MDTWLVCWAIFSLLKAGLTEPEVTQTPSHQVTQMGQEVILRCVPIS |
| | NHLYFYWYRQILGQKVEFLVSFYNNEISEKSEIFDDQFSVERPDGS |
| | NFTLKIRSTKLEDSAMYFCAITGRVSYEQYFGPGTRLTVTEdlnkvfpp |
| | evavfepskaeiahtqkatlvclatgffpdhvelswwvngkevhsgvstdpqplkeqpalndsryclss |
| | rlrvsatfwqnprnhfrcqvqfyglsendewtqdrakpvtqivsaeawgradcgitsasyhqgvlsatil |
| | yeillgkatlyavlvsalvlmamvkrkdfgsgrakrsgsg (SEQ ID NO: 36) |
| Complete Beta | ATACCTGGCTCGTGTGTTGGGCCATCTTTAGCCTGCTGAAGGCC |
| and Alpha | GGACTGACCGAGCCTGAAGTGACCCAGACTCCAAGCCATCAAGT |
| ORF DNA | GACTCAGATGGGGCAAGAAGTCATTCTGCGTTGCGTGCCCATCA |
| Sequence (The | GCAACCACCTGTACTTTTATTGGTATCGCCAGATCCTGGGCCAG |
| underlined | AAAGTGGAATTCCTGGTGTCCTTCTACAACAATGAGATCTCCG |
| italic region in | AGAAGTCCGAGATCTTCGACGACCAGTTCTCCGTGGAAAGACCC |
| the “Furin- | GACGGCAGCAACTTCACACTGAAGATCCGGTCTACCAAACTTGA |
| P2A” site | GGACTCCGCTATGTATTTTTGTGCAATCACAGGTCGCGTTTCA |
| encodes a | TATGAGCAATATTTCGGGCCGGGCACCAGGCTCACGGTCACAga |
| sequence | agatctgaacaaggtgttccctccagaggtggccgtgttcgagccttctaaggccgagatcgcccacacac |
| allowing for | aaaaagccaccctcgtgtgcctggccaccggctttttccccgaccacgtggaactgtcttggtgggtcaac |
| expression of | ggcaaagaggtgcactccggcgtgtcaacggatccccagcctctgaaagaacagcctgccctgaacgac |
| two | agccggtactgcctgagctccagactgagagtgtccgccaccttctggcagaacccccggaaccacttca |
| polypeptide | gatgccaggtgcagttttacggcctgagcgagaacgacgagtggacccaggacagagccaagcccgtg |
| chains in a | acacaaatcgtgtctgccgaagcctggggaagagccgattgcggcatcaccagcgcctcctatcaccagg |
| single | gcgtgctgagcgccacaatcctgtacgaaatcctgctgggcaaggccaccctgtacgccgtgctggtgtct |
| cassette”) | gctctggtgctgatggccatggtcaagcggaaggactttggcagcggcagagccaaaaggtccgggagc |
| | ggtGCGACAAACTTTAGCCTGTTGAAACAAGCCGGCGACGTTGAAGAG |
| | AACCCCGGACCTATGCTGCTGATCACCTCCATGCTGGTGCTGTGG |
| | ATGCAGCTGAGCCAAGTGAACGGCCAGCAAGTGATGCAGATCC |
| | CTCAGTACCAGCACGTGCAAGAAGGCGAGGACTTCACCACCTAC |
| | TGCAACAGCAGCACCACACTGAGCAACATCCAGTGGTACAAGC |
| | AGCGGCCTGGCGGACACCCTGTGTTTCTGATCCAGCTGGTCAAG |
| | TCCGGCGAAGTGAAGAAGCAGAAGCGGCTGACCTTCCAGTTCG |
| | GCGAGGCCAAGAAGAACAGCAGCCTGCACATCACCGCCACACA |
| | GACCACAGATGTGGGCACCTACTTCTGCGCTGGCATCGGTAGC |
| | AGCAACACCGGTAAGCTCATCTTTGGGCAAGGGACAACTTTA |
| | CAAGTAAAACCAGacatccagaaccccgaccccgccgtgtaccagctgagggactccaagt |
| | ccagcgacaagagcgtgtgtctgtttacggacttcgacagccagaccaacgtgagtcaaagcaaggaca |
| | gcgacgtctacataacggataagaccgtgctggacatgcggagcatggacttcaagagcaacagcgccg |
| | tggcctggtccaacaagagcgacttcgcctgcgccaacgccttcaacaacagcatcatccccgaggacac |
| | cttcttccccagcagcgacgtgccctgcgacgtgaaactggtggagaagtccttcgagacagacaccaat |
| | ctgaactttcagaacctgctggtgatcgtgctgcggattctgctgctgaaagtggccggcttcaatctgctga |
| | tgaccctgcggctgtggagc (SEQ ID NO: 37) |
| Complete Beta | MDTWLVCWAIFSLLKAGLTEPEVTQTPSHQVTQMGQEVILRCVPIS |
| and Alpha | NHLYFYWYRQILGQKVEFLVSFYNNEISEKSEIFDDQFSVERPDGS |
| ORF Protein | NFTLKIRSTKLEDSAMYFCAITGRVSYEQYFGPGTRLTVTEdlnkvfpp |
| Sequence (The | evavfepskaeiahtqkatlvclatgffpdhvelswwvngkevhsgvstdpqplkeqpalndsryclss |
| underlined | rlrvsatfwqnprnhfrcqvqfyglsendewtqdrakpvtqivsaeawgradcgitsasyhqgvlsatil |
| italic region in | yeillgkatlyavlvsalvlmamvkrkdfgsgrakrsgsgATNFSLLKQAGDVEENPGPM |
| the “Furin- | LLITSMLVLWMQLSQVNGQQVMQIPQYQHVQEGEDFTTYCNSSTT |
| P2A” site | LSNIQWYKQRPGGHPVFLIQLVKSGEVKKQKRLTFQFGEAKKNSS |
| allows | LHITATQTTDVGTYFCAGIGSSNTGKLIFGQGTTLQVKPDiqnpdpav |
| expression of | yqlrdskssdksvclftdfdsqtnvsqskdsdvyitdktvldmrsmdfksnsavawsnksdfacanaf |
| two | nnsiipedtffpssdvpcdvklveksfetdtnlnfqnllvivlrilllkvagfnllmtlrlws (SEQ ID |
| polypeptide | NO: 38) |
| chains in a | |
| single | |
| cassette”)”) | |

* Table 1 provides, in part, representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well-known IMGT nomenclature described herein. Similarly, CDR1 and CDR2 of TRAV and TRBV are well-known in the art since they are based on well-known and annotated TRAV and TRBV sequences (e.g., as annotated in databases like IMGT available at imt.org and IEDB available at iedb.org).

TABLE 2

| | |
|---|---|
| Representative | Atgtctcttgagcagaggagtctgcactgcaagcctgaggaagcccttgaggcccaacaagaggccctg |
| Human | ggcctggtgtgtgtgcaggctgccacctcctcctcctctcctctggtcctgggcaccctggaggaggtgcc |
| MAGEA1 | cactgctgggtcaacagatcctccccagagtcctcagggagcctccgcctttcccactaccatcaacttcac |
| cDNA | tcgacagaggcaacccagtgagggttccagcagccgtgaagaggaggggccaagcacctcttgtatcct |
| sequence | ggagtccttgttccgagcagtaatcactaagaaggtggctgatttggttggttttctgctcctcaaatatcgag |
| | ccagggagccagtcacaaaggcagaaatgctggagagtgtcatcaaaaattacaagcactgttttcctgag |
| | atcttcggcaaagcctctgagtccttgcagctggtctttggcattgacgtgaaggaagcagaccccaccgg |
| | ccactcctatgtccttgtcacctgcctaggtctctcctatgatggcctgctgggtgataatcagatcatgccca |
| | agacaggcttcctgataattgtcctggtcatgattgcaatggagggcggccatgctcctgaggaggaaatct |
| | gggaggagctgagtgtgatggaggtgtatgatgggagggagcacagtgcctatggggagcccaggaag |
| | ctgctcacccaagatttggtgcaggaaaagtacctggagtaccggcaggtgccggacagtgatcccgcac |
| | gctatgagttcctgtggggtccaagggccctcgctgaaaccagctatgtgaaagtccttgagtatgtgatca |
| | aggtcagtgcaagagttcgctttttcttcccatccctgcgtgaagcagctttgagagaggaggaagagg |
| | gagtctga (SEQ ID NO: 39) |
| Representative | MSLEQRSLHCKPEEALEAQQEALGLVCVQAATSSSSPLVLGTLEEV |
| Human | PTAGSTDPPQSPQGASAFPTTINFTRQRQPSEGSSSREEEGPSTSCILE |
| MAGEA1 | SLFRAVITKKVADLVGFLLLKYRAREPVTKAEMLESVIKNYKHCFP |
| protein | EIFGKASESLQLVFGIDVKEADPTGHSYVLVTCLGLSYDGLLGDNQI |
| sequence | MPKTGFLIIVLVMIAMEGGHAPEEEIWEELSVMEVYDGREHSAYGE |
| | PRKLLTQDLVQEKYLEYRQVPDSDPARYEFLWGPRALAETSYVKV |
| | LEYVIKVSARVRFFFPSLREAALREEEEGV* (SEQ ID NO: 40) |
| Representative | Atgcgcgtcatggctccacgcgccctcctcctgctgctctcagggggcctggccctgaccgagacctgg |
| Human | gcctgctcccactccatgcgctatttcgacaccgccgtgtcccgcccaggccgcggtgagccacgcttcat |
| HLA-C*07:02 | ctcagtgggctacgtggacgacactcagttcgtgcgcttcgacagcgacgccgccagtcctcgcgggga |
| DNA | gccacgcgcaccatgggtggagcaggaggggcctgagtattgggaccgcgagacacagaagtacaag |
| sequence | cgccaggcacaggctgaccgcgtgagcctgcgcaacctgcgcggctactacaaccagagcgaggacg |
| | ggtctcacaccctccagcgcatgtctggctgcgacctggggcctgacgggcgcctcctccgcgggtatga |
| | ccagtccgcctacgacggcaaggattacatcgccctgaacgaggacctgcgctcctggaccgccgctga |
| | caccgccgctcagatcacccagcgcaagttggaggctgcccgcgccgctgagcagctgcgcgcctacct |
| | ggagggcacatgcgtggagtggctccgccgctacctggagaacgggaaggagaccctgcagcgcgca |
| | gaaccaccaaagacacacgtgacccaccaccctctctctgaccatgaggccaccctgcgctgctgggcc |
| | ctgggcttctaccctgctgagatcacactgacctggcagcgcgatggggaggaccagacccaggacacc |
| | gagcttgtggagacccgcccagcaggtgatggcaccttccagaagtgggcagctgtggtggtgccttctg |
| | ggcaagagcagcgctacacatgccatatgcagcacgaggggctgcaagagccactcaccctgagctgg |
| | gagccatcttcccagccaaccatcccaatcatgggcatcgttgctggcctggctgtcctggttgtcctggctg |
| | tccttggtgctgtggtcaccgctatgatgtgtcgccgcaagagctcaggtgggaaaggtgggagctgctct |
| | caggctgcatgcagcaacagtgcccagggctctgatgagtctctcatcacttgtaaagcctga (SEQ ID |
| | NO: 41) |
| Representative | MRVMAPRALLLLLSGGLALTETWACSHSMRYFDTAVSRPGRGEPR |
| Human | FISVGYVDDTQFVRFDSDAASPRGEPRAPWVEQEGPEYWDRETQK |
| HLA-C*07:02 | YKRQAQADRVSLRNLRGYYNQSEDGSHTLQRMSGCDLGPDGRLL |
| protein | RGYDQSAYDGKDYIALNEDLRSWTAADTAAQITQRKLEAARAAE |
| sequence | QLRAYLEGTCVEWLRRYLENGKETLQRAEPPKTHVTHHPLSDHEA |
| | TLRCWALGFYPAEITLTWORDGEDQTQDTELVETRPAGDGTFQKW |
| | AAVVVPSGQEQRYTCHMQHEGLQEPLTLSWEPSSQPTIPIMGIVAG |
| | LAVLVVLAVLGAVVTAMMCRRKSSGGKGGSCSQAACSNSAQGSD |
| | ESLITCKA* (SEQ ID NO: 42) |
| Representative | tggaagggctaattcactcccaaagaagacaagatatccttgatctgtggatctaccacacacaaggctact |
| Vector (the | tccctgattagcagaactacacaccagggccaggggtcagatatccactgacctttggatggtgctacaag |
| TCR- | ctagtaccagttgagccagataaggtagaagaggccaataaaggagagaacaccagcttgttacaccctg |
| encoding | tgagcctgcatgggatggatgacccggagagagaagtgttagagtggaggtttgacagccgcctagcattt |
| protein of | catcacgtggcccgagagctgcatccggagtacttcaagaactgctgatatcgagcttgctacaagggact |
| which can be | ttccgctggggactttccagggaggcgtggcctgggcgggactggggagtggcgagccctcagatcctg |
| interchanged | catataagcagctgctttttgcctgtactgggtctctctggttagaccagatctgagcctgggagctctctggc |
| with any TCR | taactagggaacccactgcttaagcctcaataaagcttgccttgagtgcttcaagtagtgtgtgcccgtctgtt |
| sequence of | gtgtgactctggtaactagagatccctcagacccttttagtcagtgtggaaaatctctagcagtggcgcccga |
| interest): | acagggacttgaaagcgaaagggaaaccagaggagctctctcgacgcaggactcggcttgctgaagcg |
| pTSLV102- | cgcacggcaagaggcgaggggcggcgactggtgagtacgccaaaaattttgactagcggaggctagaa |
| MSCV-HA1- | ggagagagatgggtgcgagagcgtcagtattaagcgggggagaattagatcgcgatgggaaaaaattcg |
| 10-30- | gttaaggccagggggaaagaaaaaatataaattaaaacatatagtatgggcaagcagggagctagaacg |
| MGTM-Q- | attcgcagttaatcctggcctgttagaaacatcagaaggctgtagacaaatactgggacagctacaaccatc |
| CD8 | ccttcagacaggatcagaagaacttagatcattatataatacagtagcaaccctctattgtgtgcatcaaagg |
| | atagagataaaagacaccaaggaagctttagacaagatagaggaagagcaaaacaaaagtaagaccacc |
| | gcacagcaagcggccggccgctgatcttcagacctggaggaggagatatgagggacaattggagaagt |
| | gaattatataaatataaagtagtaaaaattgaaccattaggagtagcacccaccaaggcaaagagaagagt |
| | ggtgcagagagaaaaaagagcagtgggaataggagctttgttccttgggttcttgggagcagcaggaagc |
| | actatgggcgcagcgtcaatgacgctgacggtacaggccagacaattattgtctggtatagtgcagcagca |
| | gaacaatttgctgagggctattgaggcgcaacagcatctgttgcaactcacagtctggggcatcaagcagc |
| | tccaggcaagaatcctggctgtggaaagatacctaaaggatcaacagctcctggggatttggggttgctct |
| | ggaaaactcatttgcaccactgctgtgccttggaatgctagttggagtaataaatctctggaacagatttgga |
| | atcacacgacctggatggagtgggacagagaaattaacaattacacaagcttaatacactccttaattgaag |
| | aatcgcaaaaccagcaagaaaagaatgaacaagaattattggaattagataaatgggcaagtttgtggaatt |
| | ggtttaacataacaaattggctgtggtatataaaattattcataatgatagtaggaggcttggtaggtttaagaa |
| | tagtttttgctgtactttctatagtgaatagagttaggcagggatattcaccattatcgtttcagacccacctccc |
| | aaccccgaggggacccgacaggcccgaaggaatagaagaagaaggtggagagagagacagagaca |
| | gatccattcgattagtgaacggatctcgacggtatcgccgaattaattcacaaatggcagtattcatccacaat |
| | tttaaaagaaaaggggggattggggggtacagtgcaggggaaagaatagtagacataatagcaacagac |
| | atacaaactaaagaattacaaaaacaaattacaaaaattcaaaattttcgggtttattacaggCGcGCcag |
| | agatccagtttggacCTgcAGGTGAAAGACCCCACCTGTAGGTTTGGCA |
| | AGtTAGCTTAAGTAACGCCATTTTGCAAGGCATGGAAAATAC |
| | ATAACTGAGAATAGAGAAGTTCAGATCAAGGTTAGGAACAG |
| | AGAGACAGCAGAATATGGGCCAAACAGGATATCTGTGGTAA |
| | GCAGTTCCTGCCCCGGCTCAGGGCCAAGAACAGATGGTCCC |
| | CAGATGCGGTCCCGCCCTCAGCAGTTTCTAGCGAACCATCA |
| | GATGTTTCCAGGGTGCCCCAAGGACCTGAAATGACCCTGTG |
| | CCTTATTTGAACTAACCAATCAGTTtGCTTCTtGCTTCTGTTtGt |
| | GtGCTTCTGCTCCCtGAGCTCAATAAAAGAGCCCACAACCCCT |
| | CACTtGGtGgGCCAGTCCTCtGATAGACTGtGTCcCCtGGaTACCCG |
| | TAcggtaccgctagcgccaccATGGGCACCAGCCTCCTCTGCTGGATGGCC |
| | CTGTGTCTCCTGGGGGCAGATCACGCAGATACTGGAGTCTCCCAG |
| | GACCCCAGACACAAGATCACAAAGAGGGGACAGAATGTTACTTTC |
| | AGGTGTGATCCAATTTCTGAACACAACCGCCTTTATTGGTACCGC |
| | CAGACCCTGGGGCAGGGCCCAGAGTTTCTGACTTACTTCCAGAAT |
| | GAAGCTCAACTTGAAAAATCAAGGCTGCTCAGTGATCGGTTCTCT |
| | GCAGAGAGGCCTAAGGGATCTTTCTCCACCTTGGAGATCCAGCGC |
| | ACAGAGCAGGGGGACTCTGCCATGTATCTCTGTGCCAGCAGCCG |
| | CACTGCTGGAGATACTCAGTATTTTGGCCCAGGCACCCGGCTGAC |
| | AGTGCTCGAAGATCTGAACAAGGTGTTCCCTCCAGAGGTGGCCGT |
| | GTTCGAGCCTTCTaAGGCCGAGATCgccCACACaCAaAAAGCCACC |
| | CTCGTGTGCCTGGCCACCGGCTTTTTCCCCGACCACGTGGAACTG |
| | TCTTGGTGGGTCAACGGCAAAGAGGTGCACTCCGGCGTGtcAACgG |
| | ATCCCCAGCCTCTGAAAGAACAGCCTGCCCTGAACGACAGCCGGT |
| | ACTGCCTGAGCTCCAGACTGAGAGTGTCCGCCACCTTCTGGCAGA |
| | ACCCCCGGAACCACTTCAGATGCCAGGTGCAGTTTTACGGCCTGA |
| | GCGAGAACGACGAGTGGACCCAGGACAGAGCCAAGCCCGTGACA |
| | CAAATCGTGTCTGCCGAAGCCTGGGGAAGAGCCGATTGCGGCAT |
| | CACCAGCGCCTCCTATCACCAGGGCGTGCTGAGCGCCACAATCCT |
| | GTACGAAATCCTGCTGGGCAAGGCCACCCTGTACGCCGTGCTGGT |
| | GTCTGCTCTGGTGCTGATGGCCATGGTCAAGCGGAAGGACTTTGG |
| | CAGCGGCAGAGCCAAAAGGTCCGGGAGCGGTGCGACAAACTTT |
| | AGCCTGTTGAAACAAGCCGGCGACGTTGAAGAGAACCCCGGAC |
| | CTATGGAAACCCTcTTGGGCCTGCTTATCCTTTGGCTGCAGC |
| | TGCAATGGGTGAGCAGCAAACAGGAGGTGACTCAGATTCCT |
| | GCAGCTCTGAGTGTCCCAGAAGGAGAAAACTTGGTTCTCAA |
| | CTGCAGTTTCACTGATAGCGCTATTTACAACCTCCAGTGGTT |
| | TAGGCAGGACCCTGGGAAAGGCCTCACATCTCTGTTGCTTAT |
| | TCAGTCAAGTCAGAGAGAGCAAACAAGTGGACGCCTTAATG |
| | CCTCTCTGGATAAATCATCAGGACGCAGTACTCTTTACATTG |
| | CAGCTTCTCAGCCTGGTGATTCAGCCACCTACCTGTGCGCTG |
| | TGAGGGGTGGTACCTCAGGAACCTACAAATACATCTTTGGA |
| | ACAGGCACCAGGCTGAAGGTTCTTGCAAACATCCAGAACCC |
| | CGACCCCGCCGTGTACCAGCTGAGGGACTCCAAGTCCAGCG |
| | ACAAGAGCGTGTGTCTGTTTACGGACTTCGACAGCCAGACC |
| | AACGTGAGTCAAAGCAAGGACAGCGACGTCTACATAACGGA |
| | TAAGACCGTGCTGGACATGCGGAGCATGGACTTCAAGAGCA |
| | ACAGCGCCGTGGCCTGGTCCAACAAGAGCGACTTCGCCTGC |
| | GCCAACGCCTTCAACAACAGCATCATCCCCGAGGACACCTTC |
| | TTCCCCAGCAGCGACGTGCCCTGCGACGTGAAACTGGTGGA |
| | GAAGTCCTTCGAGACAGACACCAATCTGAACTTTCAGAACCT |
| | GCTGGTGATCGTGCTGCGGATTCTGCTGCTGAAAGTGGCCG |
| | GCTTCAATCTGCTGATGACCCTGCGGCTGTGGAGCAGCAGG |
| | GCTAAGAGGTCCGGCAGCGGAGCCACCAATTTTTCCCTGCTGAA |
| | ACAGGCTGGTGACGTGGAAGAAAACCCTGGCCCCATGGCGCTG |
| | CCCGTCACCGCGCTGCTGCTGCCCCTGGCGCTGCTGTTACACGCC |
| | GCTCGGCCAGAGCTTCCCACCCAGGGCACATTCTCCAACGTGTCCA |
| | CCAATGTGTCGGGAGGCGGCGGATCGTCCCAGTTCAGAGTGTCCCC |
| | TCTGGACCGCACCTGGAACCTGGGCGAGACCGTGGAGCTGAAATGT |
| | CAGGTCCTGCTGAGCAACCCGACCTCCGGGTGCAGTTGGCTGTTCC |
| | AGCCGCGTGGTGCTGCCGCAAGCCCTACGTTCCTGCTTTACCTGAG |
| | CCAGAACAAGCCCAAGGCGGCCGAGGGCCTGGACACCCAGAGATT |
| | CTCCGGCAAGCGCCTGGGGGACACATTCGTGCTTACTTTGAGCGAT |
| | TTCCGCAGAGAGAACGAGGGCTACTATTTCTGTTCGGCGCTGAGCAA |
| | TTCCATCATGTATTTCAGCCACTTTGTGCCAGTGTTCCTGCCTGCCAA |
| | GCCTACCACAACACCAGCTCCCCGTCCCCCGACTCCGGCGCCTACC |
| | ATCGCGAGTCAACCGTTGAGCCTGAGGCCTGAGGCTTGTCGGCCCG |
| | CTGCGGGGGGTGCCGTCCACACCAGGGGCCTCGACTTTGCGTGCG |
| | ACATCTATATTTGGGCGCCTCTGGCGGGTACCTGCGGGGTGCTGCT |
| | GCTGTCATTGGTGATTACCCTGTACTGCAATCACCGCAACCGCCGGC |
| | GGGTCTGTAAGTGCCCACGGCCTGTGGTCAAGTCCGGTGACAAACC |
| | GTCGCTCTCGGCTCGCTACGTGCGCGCTAAGCGCAGCGGTTCCGG |
| | GGCCACCAACTTTTCATTGCTGAAGCAGGCCGGTGATGTGGAGG |
| | AGAATCCAGGGCCCATGCGCCCCAGGCTTTGGCTCCTTCTTGCT |
| | GCTCAGCTCACTGTCTTGCATGGCAACTCCGTTCTGCAGCAGACT |
| | CCCGCCTACATCAAGGTGCAGACGAACAAGATGGTGATGCTGTC |
| | ATGCGAGGCCAAGATCTCTCTTTCAAATATGAGAATTTATTGGC |
| | TACGACAGCGCCAGGCCCCCTCCAGCGACAGCCACCACGAGTTC |
| | CTGGCGCTTTGGGATTCTGCTAAAGGCACCATCCATGGAGAGGA |
| | GGTGGAACAGGAGAAGATAGCTGTCTTCCGCGACGCATCCCGCT |
| | TCATCCTGAACCTGACCAGCGTGAAGCCGGAGGACAGCGGCATC |
| | TACTTCTGTATGATCGTTGGCTCCCCCGAGCTGACCTTCGGCAAA |
| | GGCACCCAGCTGTCCGTGGTGGACTTCCTGCCCACCACAGCCCA |
| | GCCAACCAAGAAATCCACCCTCAAGAAGCGCGTGTGCCGACTGC |
| | CCCGCCCTGAAACCCAGAAGGGCCCTCTGTGCTCCCCCATCACC |
| | CTTGGACTGCTGGTGGCGGGAGTCCTGGTGCTGCTCGTATCTCTG |
| | GGTGTCGCCATCCACCTGTGCTGCCGCCGCCGCCGCGCCCGCCT |
| | GAGGTTTATGAAACAGTTTTACAAGTGATAAatcgatagatcctaatcaacct |
| | ctggattacaaaatttgtgaaagattgactggtattcttaactatgttgctccttttacgctatgtggatacgctgc |
| | tttaatgcctttgtatcatgctattgcttcccgtatggctttcattttctcctccttgtataaatcctggttgctgt |
| | ctctttatgaggagttgtggcccgttgtcaggcaacgtggcgtggtgtgcactgtgtttgctgacgcaacccccac |
| | tggttggggcattgccaccacctgtcagctcctttccgggactttcgctttccccctccctattgccacggcg |
| | gaactcatcgccgcctgccttgcccgctgctggacaggggctcggctgttgggcactgacaattccgtggt |
| | gttgtcggggaaatcatcgtcctttccttggctgctcgcctgtgttgccacctggattctgcgcgggacgtcct |
| | tctgctacgtcccttcggccctcaatccagcggaccttccttcccgcggcctgctgccggctctgcggcctc |
| | ttccgcgtcttcgccttcgccctcagacgagtcggatctccctttgggccgcctccccgcctgagatccttta |
| | agaccaatgacttacaaggcagctgtagatcttagccactttttaaaagaaaaggggggactggaagggct |
| | aattcactcccaacgaagacaagatctgctttttgcttgtactgggtctctctggttagaccagatctgagcct |
| | gggagctctctggctaactagggaacccactgcttaagcctcaataaagcttgccttgagtgcttcaagtagt |
| | gtgtgcccgtctgttgtgtgactctggtaactagagatccctcagacccttttagtcagtgtggaaaatctcta |
| | gcagtagtagttcatgtcatcttattattcagtatttataacttgcaaagaaatgaatatcagagagtgagaggc |
| | ccgggttaattaaggaaagggctagatcattcttgaagacgaaagggcctcgtgatacgcctatttttatagg |
| | ttaatgtcatgataataatggtttcttagacgtcaggtggcacttttcggggaaatgtgcgcggaacccctattt |
| | gtttatttttctaaatacattcaaatatgtatccgctcatgagacaataaccctgataaatgcttcaataatattga |
| | aaaaggaagagtatgagtattcaacatttccgtgtcgcccttattcccttttttgcggcattttgccttcctgttttt |
| | gctcacccagaaacgctggtgaaagtaaaagatgctgaagatcagttgggtgcacgagtgggttacatcg |
| | aactggatctcaacagcggtaagatccttgagagttttcgccccgaagaacgttttccaatgatgagcactttt |
| | aaagttctgctatgtggcgcggtattatcccgtgttgacgccgggcaagagcaactcggtcgccgcataca |
| | ctattctcagaatgacttggttgagtactcaccagtcacagaaaagcatcttacggatggcatgacagtaaga |
| | gaattatgcagtgctgccataaccatgagtgataacactgcggccaacttacttctgacaacgatcggagga |
| | ccgaaggagctaaccgcttttttgcacaacatgggggatcatgtaactcgccttgatcgttgggaaccggag |
| | ctgaatgaagccataccaaacgacgagcgtgacaccacgatgcctgtagcaatggcaacaacgttgcgc |
| | aaactattaactggcgaactacttactctagcttcccggcaacaattaatagactggatggaggcggataaa |
| | gttgcaggaccacttctgcgctcggcccttccggctggctggtttattgctgataaatctggagccggtgag |
| | cgtgggtctcgcggtatcattgcagcactggggccagatggtaagccctcccgtatcgtagttatctacacg |
| | acggggagtcaggcaactatggatgaacgaaatagacagatcgctgagataggtgcctcactgattaagc |
| | attggtaactgtcagaccaagtttactcatatatactttagattgatttaaaacttcatttttaatttaaaaggatcta |
| | ggtgaagatcctttttgataatctcatgaccaaaatcccttaacgtgagttttcgttccactgagcgtcagaccc |
| | cgtagaaaagatcaaaggatcttcttgagatcctttttttctgcgcgtaatctgctgcttgcaaacaaaaaaac |
| | caccgctaccagcggtggtttgtttgccggatcaagagctaccaactctttttccgaaggtaactggcttcag |
| | cagagcgcagataccaaatactgttcttctagtgtagccgtagttaggccaccacttcaagaactctgtagca |
| | ccgcctacatacctcgctctgctaatcctgttaccagtggctgctgccagtggcgataagtcgtgtcttaccg |
| | ggttggactcaagacgatagttaccggataaggcgcagcggtcgggctgaacggggggttcgtgcacac |
| | agcccagcttggagcgaacgacctacaccgaactgagatacctacagcgtgagctatgagaaagcgcca |
| | cgcttcccgaagggagaaaggcggacaggtatccggtaagcggcagggtcggaacaggagagcgcac |
| | gagggagcttccagggggaaacgcctggtatctttatagtcctgtcgggtttcgccacctctgacttgagcg |
| | tcgatttttgtgatgctcgtcaggggggcggagcctatggaaaaacgccagcaacgcggcctttttacggtt |
| | cctggccttttgctggccttttgctcacatgttctttcctgcgttatccCCTGATTCTGTGGATAA |
| | CCGTATTACCGCCTTTGAGTGAGCTGATACCGCTCGCCGCAGCC |
| | GAACGACCGAGCGCAGCGAGTCAGTGAGCGAGGAAGCGGAAGA |
| | GCGCCCAATACGCAAACCGCCTCTCCCCGCGCGTTGGCCGATTC |
| | ATTAATGCAGCAAGCTCATGGCTGACTAATTTTTTTTATTTATGC |
| | AGAGGCCGAGGCCGCCTCGGCCTCTGAGCTATTCCAGAAGTAGT |
| | GAGGAGGCTTTTTTGGAGGCCTAGGCTTTTGCAAAAAGCTCCCC |
| | GTGGCACGACAGGTTTCCCGACTGGAAAGCGGGCAGTGAGCGC |
| | AACGCAATTAATGTGAGTTAGCTCACTCATTAGGCACCCCAGGC |
| | TTTACACTTTATGCTTCCGGCTCGTATGTTGTGTGGAATTGTGAG |
| | CGGATAACAATTTCACACAGGAAACAGCTATGACATGATTACGA |
| | ATTTCACAAATAAAGCATTTTTTTCACTGCATTCTAGTTGTGGTT |
| | TGTCCAAACTCATCAATGTATCTTATCATGTCTGGATCAACTGGA |
| | TAACTCAAGCTAACCAAAATCATCCCAAACTTCCCACCCCATAC |
| | CCTATTACCACTGCCAATTACCTGTGGTTTCATTTACTCTAAACC |
| | TGTGATTCCTCTGAATTATTTTCATTTTAAAGAAATTGTATTTGTT |
| | AAATATGTACTACAAACTtagtagt (SEQ ID NO: 43) |
| Representative | Atgcctcccgttccaggcgttccattccgcaacgttgacaacgactccccgacctcagttgagttagaaga |
| Human | ctgggtagatgcacagcatcccacagatgaggaagaggaggaagcctcctccgcctcttccactttgtactt |
| MAGEC2 | agtattttccccctcttctttctccacatcctcttctctgattcttggtggtcctgaggaggaggaggtgccctct |
| cDNA | ggtgtgataccaaatcttaccgagagcattcccagtagtcctccacagggtcctccacagggtccttcccag |
| sequence | agtcctctgagctcctgctgctcctctttttcatggagctcattcagtgaggagtccagcagccagaaaggg |
| | gaggatacaggcacctgtcagggcctgccagacagtgagtcctctttcacatatacactagatgaaaaggt |
| | ggccgagttagtggagttcctgctcctcaaatacgaagcagaggagcctgtaacagaggcagagatgctg |
| | atgattgtcatcaagtacaaagattactttcctgtgatactcaagagagcccgtgagttcatggagcttctttttg |
| | gccttgccctgatagaagtgggccctgaccacttctgtgtgtttgcaaacacagtaggcctcaccgatgagg |
| | gtagtgatgatgagggcatgcccgagaacagcctcctgattattattctgagtgtgatcttcataaagggcaa |
| | ctgtgcctctgaggaggtcatctgggaagtgctgaatgcagtaggggtatatgctgggagggagcacttcg |
| | tctatggggagcctagggagctcctcactaaagtttgggtgcagggacattacctggagtatcgggaggtg |
| | ccccacagttctcctccatattatgaattcctgtggggtccgagagcccattcagaaagcatcaagaagaaa |
| | gtactagagtttttagccaagctgaacaacactgttcctagttcctttccatcctggtacaaggatgctttgaaa |
| | gatgtggaagagagagtccaggccacaattgataccgcagatgatgccactgtcatggccagtgaaagcc |
| | tcagtgtcatgtccagcaacgtctccttttctgagtga (SEQ ID NO: 44) |
| Representative | MPPVPGVPFRNVDNDSPTSVELEDWVDAQHPTDEEEEEASSASSTL |
| Human | YLVFSPSSFSTSSSLILGGPEEEEVPSGVIPNLTESIPSSPPQGPPQGPS |
| MAGEC2 | QSPLSSCCSSFSWSSFSEESSSQKGEDTGTCQGLPDSESSFTYTLDEK |
| protein | VAELVEFLLLKYEAEEPVTEAEMLMIVIKYKDYFPVILKRAREFME |
| sequence | LLFGLALIEVGPDHFCVFANTVGLTDEGSDDEGMPENSLLIIILSVIFI |
| (Representative, | KGNCASEEVIWEVLNAVGVYAGREHFVYGEPRELLTKVWVQGHY |
| non-limiting | LEYREVPHSSPPYYEFLWGPRAHSESIKKKVLEFLAKLNNTVPSSFP |
| epitopes | SWYKDALKDVEERVQATIDTADDATVMASESLSVMSSNVSFSE |
| underlined) | (SEQ ID NO: 45) |
| Representative | Atgctggtcatggcgccccgaaccgtcctcctgctgctctcggcggccctggccctgaccgagacctgg |
| Human | gccggctcccactccatgaggtatttctacacctccgtgtcccggcccggccgggggagccccgcttcat |
| HLA-B*07:02 | ctcagtgggctacgtggacgacacccagttcgtgaggttcgacagcgacgccgcgagtccgagagagg |
| DNA | agccgcgggcgccgtggatagagcaggaggggccggagtattgggaccggaacacacagatctacaa |
| sequence | ggcccaggcacagactgaccgagagagcctgcggaacctgcgoggctactacaaccagagcgaggcc |
| | gggtctcacaccctccagagcatgtacggctgcgacgtggggccggacgggcgcctcctccgcgggca |
| | tgaccagtacgcctacgacggcaaggattacatcgccctgaacgaggacctgcgctcctggaccgccgc |
| | ggacacggcggctcagatcacccagcgcaagtgggaggcggcccgtgaggcggagcagcggagagc |
| | ctacctggagggcgagtgcgtggagtggctccgcagatacctggagaacgggaaggacaagctggagc |
| | gcgctgaccccccaaagacacacgtgacccaccaccccatctctgaccatgaggccaccctgaggtgct |
| | gggccctgggtttctaccctgcggagatcacactgacctggcagcgggatggcgaggaccaaactcagg |
| | acactgagcttgtggagaccagaccagcaggagatagaaccttccagaagtgggcagctgtggtggtgc |
| | cttctggagaagagcagagatacacatgccatgtacagcatgaggggctgccgaagcccctcaccctga |
| | gatgggagccgtcttcccagtccaccgtccccatcgtgggcattgttgctggcctggctgtcctagcagttgt |
| | ggtcatcggagctgtggtcgctgctgtgatgtgtaggaggaagagttcaggtggaaaaggagggagctac |
| | tctcaggctgcgtgcagcgacagtgcccagggctctgatgtgtctctcacagcttga (SEQ ID NO: |
| | 46) |
| Representative | MLVMAPRTVLLLLSAALALTETWAGSHSMRYFYTSVSRPGRGEPR |
| Human | FISVGYVDDTQFVRFDSDAASPREEPRAPWIEQEGPEYWDRNTQIY |
| HLA-B*07:02 | KAQAQTDRESLRNLRGYYNQSEAGSHTLQSMYGCDVGPDGRLLR |
| protein | GHDQYAYDGKDYIALNEDLRSWTAADTAAQITQRKWEAAREAEQ |
| sequence | RRAYLEGECVEWLRRYLENGKDKLERADPPKTHVTHHPISDHEAT |
| | LRCWALGFYPAEITLTWQRDGEDQTQDTELVETRPAGDRTFQKWA |
| | AVVVPSGEEQRYTCHVQHEGLPKPLTLRWEPSSQSTVPIVGIVAGL |
| | AVLAVVVIGAVVAAVMCRRKSSGGKGGSYSQAACSDSAQGSDVS |
| | LTA (SEQ ID NO: 47) |
| Representative | MHGDTPTLHEYMLDLQPETTDLYCYEQLNDSSEEEDEIDGPAGQA |
| HPV16 E7 | EPDRAHYNIVTFCCKCDSTLRLCVQSTHVDIRTLEDLLMGTLGIVCP |
| protein | ICSQKP (SEQ ID NO: 48) |
| sequence | |
| (Representative, | |
| non-limiting | |
| epitopes | |
| underlined) | |
| Representative | ATGGCCGTCATGGCGCCCCGAACCCTCGTCCTGCTACTCTCGGG |
| Human | GGCTCTGGCCCTGACCCAGACCTGGGCGGGCTCTCACTCCATGA |
| HLA-A*02:01 | GGTATTTCTTCACATCCGTGTCCCGGCCCGGCCGCGGGGAGCCC |
| DNA | CGCTTCATCGCAGTGGGCTACGTGGACGACACGCAGTTCGTGCG |
| sequence | GTTCGACAGCGACGCCGCGAGCCAGAGGATGGAGCCGCGGGCG |
| (CDS) | CCGTGGATAGAGCAGGAGGGTCCGGAGTATTGGGACGGGGAGA |
| | CACGGAAAGTGAAGGCCCACTCACAGACTCACCGAGTGGACCT |
| | GGGGACCCTGCGCGGCTACTACAACCAGAGCGAGGCCGGTTCTC |
| | ACACCGTCCAGAGGATGTATGGCTGCGACGTGGGGTCGGACTGG |
| | CGCTTCCTCCGCGGGTACCACCAGTACGCCTACGACGGCAAGGA |
| | TTACATCGCCCTGAAAGAGGACCTGCGCTCTTGGACCGCGGCGG |
| | ACATGGCAGCTCAGACCACCAAGCACAAGTGGGAGGCGGCCCA |
| | TGTGGCGGAGCAGTTGAGAGCCTACCTGGAGGGCACGTGCGTG |
| | GAGTGGCTCCGCAGATACCTGGAGAACGGGAAGGAGACGCTGC |
| | AGCGCACGGACGCCCCCAAAACGCATATGACTCACCACGCTGTC |
| | TCTGACCATGAAGCCACCCTGAGGTGCTGGGCCCTGAGCTTCTA |
| | CCCTGCGGAGATCACACTGACCTGGCAGCGGGATGGGGAGGAC |
| | CAGACCCAGGACACGGAGCTCGTGGAGACCAGGCCTGCAGGGG |
| | ATGGAACCTTCCAGAAGTGGGCGGCTGTGGTGGTGCCTTCTGGA |
| | CAGGAGCAGAGATACACCTGCCATGTGCAGCATGAGGGTTTGCC |
| | CAAGCCCCTCACCCTGAGATGGGAGCCGTCTTCCCAGCCCACCA |
| | TCCCCATCGTGGGCATCATTGCTGGCCTGGTTCTCTTTGGAGCTG |
| | TGATCACTGGAGCTGTGGTCGCTGCTGTGATGTGGAGGAGGAAG |
| | AGCTCAGATAGAAAAGGAGGGAGCTACTCTCAGGCTGCAAGCA |
| | GTGACAGTGCCCAGGGCTCTGATGTGTCTCTCACAGCTTGTAAA |
| | GTGTGA (SEQ ID NO: 49) |
| Representative | MAVMAPRTLVLLLSGALALTQTWAGSHSMRYFFTSVSRPGRGEPR |
| Human | FIAVGYVDDTQFVRFDSDAASQRMEPRAPWIEQEGPEYWDGETRK |
| HLA-A*02:01 | VKAHSQTHRVDLGTLRGYYNQSEAGSHTVQRMYGCDVGSDWRFL |
| protein | RGYHQYAYDGKDYIALKEDLRSWTAADMAAQTTKHKWEAAHVA |
| sequence | EQLRAYLEGTCVEWLRRYLENGKETLQRTDAPKTHMTHHAVSDH |
| | EATLRCWALSFYPAEITLTWQRDGEDQTQDTELVETRPAGDGTFQ |
| | KWAAVVVPSGQEQRYTCHVQHEGLPKPLTLRWEPSSQPTIPIVGIIA |
| | GLVLFGAVITGAVVAAVMWRRKSSDRKGGSYSQAASSDSAQGSD |
| | VSLTACKV (SEQ ID NO: 50) |
| Representative | tggaagggctaattcactcccaaagaagacaagatatccttgatctgtggatctaccacacacaaggctact |
| Vector (the | tccctgattagcagaactacacaccagggccaggggtcagatatccactgacctttggatggtgctacaag |
| TCR- | ctagtaccagttgagccagataaggtagaagaggccaataaaggagagaacaccagcttgttacaccctg |
| encoding | tgagcctgcatgggatggatgacccggagagagaagtgttagagtggaggtttgacagccgcctagcattt |
| protein of | catcacgtggcccgagagctgcatccggagtacttcaagaactgctgatatcgagcttgctacaagggact |
| which can be | ttccgctggggactttccagggaggcgtggcctggggggactggggagtggcgagccctcagatcctg |
| interchanged | catataagcagctgctttttgcctgtactgggtctctctggttagaccagatctgagcctgggagctctctggc |
| with any TCR | taactagggaacccactgcttaagcctcaataaagcttgccttgagtgcttcaagtagtgtgtgcccgtctgtt |
| sequence of | gtgtgactctggtaactagagatccctcagacccttttagtcagtgtggaaaatctctagcagtggcgcccga |
| interest): | acagggacttgaaagcgaaagggaaaccagaggagctctctcgacgcaggactcggcttgctgaagcg |
| pHAGE- | cgcacggcaagaggcgaggggcggcgactggtgagtacgccaaaaattttgactagcggaggctagaa |
| MSCV-E7-11- | ggagagagatgggtgcgagagcgtcagtattaagcgggggagaattagatcgcgatgggaaaaaattcg |
| 28-P2A- | gttaaggccagggggaaagaaaaaatataaattaaaacatatagtatgggcaagcagggagctagaacg |
| dnTGFbRII | attcgcagttaatcctggcctgttagaaacatcagaaggctgtagacaaatactgggacagctacaaccatc |
| | ccttcagacaggatcagaagaacttagatcattatataatacagtagcaaccctctattgtgtgcatcaaagg |
| | atagagataaaagacaccaaggaagctttagacaagatagaggaagagcaaaacaaaagtaagaccacc |
| | gcacagcaagcggccggccgctgatcttcagacctggaggaggagatatgagggacaattggagaagt |
| | gaattatataaatataaagtagtaaaaattgaaccattaggagtagcacccaccaaggcaaagagaagagt |
| | ggtgcagagagaaaaaagagcagtgggaataggagctttgttccttgggttcttgggagcagcaggaagc |
| | actatgggcgcagcgtcaatgacgctgacggtacaggccagacaattattgtctggtatagtgcagcagca |
| | gaacaatttgctgagggctattgaggcgcaacagcatctgttgcaactcacagtctggggcatcaagcagc |
| | tccaggcaagaatcctggctgtggaaagatacctaaaggatcaacagctcctggggatttggggttgctct |
| | ggaaaactcatttgcaccactgctgtgccttggaatgctagttggagtaataaatctctggaacagatttgga |
| | atcacacgacctggatggagtgggacagagaaattaacaattacacaagcttaatacactccttaattgaag |
| | aatcgcaaaaccagcaagaaaagaatgaacaagaattattggaattagataaatgggcaagtttgtggaatt |
| | ggtttaacataacaaattggctgtggtatataaaattattcataatgatagtaggaggcttggtaggtttaagaa |
| | tagtttttgctgtactttctatagtgaatagagttaggcagggatattcaccattatcgtttcagacccacctccc |
| | aaccccgaggggacccgacaggcccgaaggaatagaagaagaaggtggagagagagacagagaca |
| | gatccattcgattagtgaacggatctcgacggtatcgccgaattaattcacaaatggcagtattcatccacaat |
| | tttaaaagaaaaggggggattggggggtacagtgcaggggaaagaatagtagacataatagcaacagac |
| | atacaaactaaagaattacaaaaacaaattacaaaaattcaaaattttcgggtttattacaggCGcGCcag |
| | agatccagtttggacCTgcAGGTGAAAGACCCCACCTGTAGGTTTGGCAA |
| | GtTAGCTTAAGTAACGCCATTTTGCAAGGCATGGAAAATACATA |
| | ACTGAGAATAGAGAAGTTCAGATCAAGGTTAGGAACAGAGAGA |
| | CAGCAGAATATGGGCCAAACAGGATATCTGTGGTAAGCAGTTCC |
| | TGCCCCGGCTCAGGGCCAAGAACAGATGGTCCCCAGATGCGGTC |
| | CCGCCCTCAGCAGTTTCTAGCGAACCATCAGATGTTTCCAGGGT |
| | GCCCCAAGGACCTGAAATGACCCTGTGCCTTATTTGAACTAACC |
| | AATCAGTTtGCTTCTtGCTTCTGTTtGtGtGCTTCTGCTCCCtGAGCTC |
| | AATAAAAGAGCCCACAACCCCTCACTtGGtGgGCCAGTCCTCtGAT |
| | AGACTGtGTCcCCtGGaTACCCGTAcggtaccgctagcgccaccatggatacctggct |
| | cgtgtgttgggccatctttagcctgctgaaggccggactgaccgagcctgaagtgacccagactccaagc |
| | catcaagtgactcagatggggcaagaagtcattctgcgttgcgtgcccatcagcaaccacctgtacttttatt |
| | ggtatcgccagatcctgggccagaaagtggaattcctggtgtccttctacaacaatgagatctccgagaagt |
| | ccgagatcttcgacgaccagttctccgtggaaagacccgacggcagcaacttcacactgaagatccggtct |
| | accaaacttgaggactccgctatgtatttttgtgcAATCACAGGTCGCGTTTCATATGA |
| | GCAATATTTCGGGCCGGGCACCAGGCTCACGGTCACAGAAGATC |
| | TGAACAAGGTGTTCCCTCCAGAGGTGGCCGTGTTCGAGCCTTCT |
| | AAGGCCGAGATCGCCCACACACAAAAAGCCACCCTCGTGTGCCT |
| | GGCCACCGGCTTTTTCCCCGACCACGTGGAACTGTCTTGGTGGG |
| | TCAACGGCAAAGAGGTGCACTCCGGCGTGTCAACGGATCCCCAG |
| | CCTCTGAAAGAACAGCCTGCCCTGAACGACAGCCGGTACTGCCT |
| | GAGCTCCAGACTGAGAGTGTCCGCCACCTTCTGGCAGAACCCCC |
| | GGAACCACTTCAGATGCCAGGTGCAGTTTTACGGCCTGAGCGAG |
| | AACGACGAGTGGACCCAGGACAGAGCCAAGCCCGTGACACAAA |
| | TCGTGTCTGCCGAAGCCTGGGGAAGAGCCGATTGCGGCATCACC |
| | AGCGCCTCCTATCACCAGGGCGTGCTGAGCGCCACAATCCTGTA |
| | CGAAATCCTGCTGGGCAAGGCCACCCTGTACGCCGTGCTGGTGT |
| | CTGCTCTGGTGCTGATGGCCATGGTCAAGCGGAAGGACTTTGGC |
| | AGCGGCAGAGCCAAAAGGTCCGGGAGCGGTGCGACAAACTTTA |
| | GCCTGTTGAAAcaagccggCGACGTTGAAGAGAACCCCGGACCTatgc |
| | tgctgatcacctccatgctggtgctgtggatgcagctgagccaagtgaacggccagcaagtgatgcagatc |
| | cctcagtaccagcacgtgcaagaaggcgaggacttcaccacctactgcaacagcagcaccacactgagc |
| | aacatccagtggtacaagcagcggcctggcggacaccctgtgtttctgatccagctggtcaagtccggcg |
| | aagtgaagaagcagaagcggctgaccttccagttcggcgaggccaagaagaacagcagcctgcacatc |
| | accgccacacagaccacagatgtgggcacctacttcTGCGCTGGCATCGGTAGCAGC |
| | AACACCGGTAAGCTCATCTTTGGGCAAGGGACAACTTTACAAGT |
| | AAAACCAGacatccagaaccccgaccccgccgtgtaccagctgagggactccaagtccagcgac |
| | aagagcgtgtgtctgtttacggacttcgacagccagaccaacgtgagtcaaagcaaggacagcgacgtct |
| | acataacggataagaccgtgctggacatgcggagcatggacttcaagagcaacagcgccgtggcctggt |
| | ccaacaagagcgacttcgcctgcgccaacgccttcaacaacagcatcatccccgaggacaccttcttcccc |
| | agcagcgacgtgccctgcgacgtgaaactggtggagaagtccttcgagacagacaccaatctgaactttc |
| | agaacctgctggtgatcgtgctgcggattctgctgCTGAAAGTGGCCGGCTTCAATCT |
| | GCTGATGACCCTGCGGCTGTGGAGCAGCAGGGCTAAGAGGTCC |
| | GGCAGCGGAGCCACCAATTTTTCCCTGCTGAAACAGGCTGGTGA |
| | CGTGGAAGAAAACCCTGGCCCCATGGGTCGGGGGCTGCTCAG |
| | GGGCCTGTGGCCGCTGCACATCGTCCTGTGGACGCGTATCG |
| | CCAGCACGATCCCACCGCACGTTCAGAAGTCGGTTAATAAC |
| | GACATGATAGTCACTGACAACAACGGTGCAGTCAAGTTTCCA |
| | CAACTGTGTAAATTTTGTGATGTGAGATTTTCCACCTGTGAC |
| | AACCAGAAATCCTGCATGAGCAACTGCAGCATCACCTCCATC |
| | TGTGAGAAGCCACAGGAAGTCTGTGTGGCTGTATGGAGAAA |
| | GAATGACGAGAACATAACACTAGAGACAGTTTGCCATGACC |
| | CCAAGCTCCCCTACCATGACTTTATTCTGGAAGATGCTGCTT |
| | CTCCAAAGTGCATTATGAAGGAAAAAAAAAAGCCTGGTGAG |
| | ACTTTCTTCATGTGTTCCTGTAGCTCTGATGAGTGCAATGAC |
| | AACATCATCTTCTCAGAAGAATATAACACCAGCAATCCTGAC |
| | TTGTTGCTAGTCATATTTCAAGTGACAGGCATCAGCCTCCTG |
| | CCACCACTGGGAGTTGCCATATCTGTCATCATCATCTTCTAC |
| | TGCTACCGCGTTaaccggcagcagaagTAGTGATAAatcgatagatcctaatcaac |
| | ctctggattacaaaatttgtgaaagattgactggtattcttaactatgttgctccttttacgctatgtggatacgct |
| | gctttaatgcctttgtatcatgctattgcttcccgtatggctttcattttctcctccttgtataaatcctggttgctgtc |
| | tctttatgaggagttgtggcccgttgtcaggcaacgtggcgtggtgtgcactgtgtttgctgacgcaaccccc |
| | actggttggggcattgccaccacctgtcagctcctttccgggactttcgctttccccctccctattgccacggc |
| | ggaactcatcgccgcctgccttgcccgctgctggacaggggctcggctgttgggcactgacaattccgtg |
| | gtgttgtcggggaaatcatcgtcctttccttggctgctcgcctgtgttgccacctggattctgcgcgggacgtc |
| | cttctgctacgtcccttcggccctcaatccagcggaccttccttcccgcggcctgctgccggctctgcggcc |
| | tcttccgcgtcttcgccttcgccctcagacgagtcggatctccctttgggccgcctccccgcctgagatccttt |
| | aagaccaatgacttacaaggcagctgtagatcttagccactttttaaaagaaaaggggggactggaagggc |
| | taattcactcccaacgaagacaagatctgctttttgcttgtactgggtctctctggttagaccagatctgagcct |
| | gggagctctctggctaactagggaacccactgcttaagcctcaataaagcttgccttgagtgcttcaagtagt |
| | gtgtgcccgtctgttgtgtgactctggtaactagagatccctcagacccttttagtcagtgtggaaaatctcta |
| | gcagtagtagttcatgtcatcttattattcagtatttataacttgcaaagaaatgaatatcagagagtgagaggc |
| | ccgggttaattaaggaaagggctagatcattcttgaagacgaaagggcctcgtgatacgcctatttttatagg |
| | ttaatgtcatgataataatggtttcttagacgtcaggtggcacttttcggggaaatgtgcgcggaacccctattt |
| | gtttatttttctaaatacattcaaatatgtatccgctcatgagacaataaccctgataaatgcttcaataatattga |
| | aaaaggaagagtatgagtattcaacatttccgtgtcgcccttattcccttttttgcggcattttgccttcctgttttt |
| | gctcacccagaaacgctggtgaaagtaaaagatgctgaagatcagttgggtgcacgagtgggttacatcg |
| | aactggatctcaacagcggtaagatccttgagagttttcgccccgaagaacgttttccaatgatgagcactttt |
| | aaagttctgctatgtggcgcggtattatcccgtgttgacgccgggcaagagcaactcggtcgccgcataca |
| | ctattctcagaatgacttggttgagtactcaccagtcacagaaaagcatcttacggatggcatgacagtaaga |
| | gaattatgcagtgctgccataaccatgagtgataacactgcggccaacttacttctgacaacgatcggagga |
| | ccgaaggagctaaccgcttttttgcacaacatgggggatcatgtaactcgccttgatcgttgggaaccggag |
| | ctgaatgaagccataccaaacgacgagcgtgacaccacgatgcctgtagcaatggcaacaacgttgcgc |
| | aaactattaactggcgaactacttactctagcttcccggcaacaattaatagactggatggaggggataaa |
| | gttgcaggaccacttctgcgctcggcccttccggctggctggtttattgctgataaatctggagccggtgag |
| | cgtgggtctcgcggtatcattgcagcactggggccagatggtaagccctcccgtatcgtagttatctacacg |
| | acggggagtcaggcaactatggatgaacgaaatagacagatcgctgagataggtgcctcactgattaagc |
| | attggtaactgtcagaccaagtttactcatatatactttagattgatttaaaacttcatttttaatttaaaaggatcta |
| | ggtgaagatcctttttgataatctcatgaccaaaatcccttaacgtgagttttcgttccactgagcgtcagaccc |
| | cgtagaaaagatcaaaggatcttcttgagatcctttttttctgcgcgtaatctgctgcttgcaaacaaaaaaac |
| | caccgctaccagcggtggtttgtttgccggatcaagagctaccaactctttttccgaaggtaactggcttcag |
| | cagagcgcagataccaaatactgttcttctagtgtagccgtagttaggccaccacttcaagaactctgtagca |
| | ccgcctacatacctcgctctgctaatcctgttaccagtggctgctgccagtggcgataagtcgtgtcttaccg |
| | ggttggactcaagacgatagttaccggataaggcgcagcggtcgggctgaacggggggttcgtgcacac |
| | agcccagcttggagcgaacgacctacaccgaactgagatacctacagcgtgagctatgagaaagcgcca |
| | cgcttcccgaagggagaaaggcggacaggtatccggtaagcggcagggtcggaacaggagagcgcac |
| | gagggagcttccagggggaaacgcctggtatctttatagtcctgtcgggtttcgccacctctgacttgagcg |
| | tcgatttttgtgatgctcgtcaggggggcggagcctatggaaaaacgccagcaacgcggcctttttacggtt |
| | cctggccttttgctggccttttgctcacatgttctttcctgcgttatccCCTGATTCTGTGGATAA |
| | CCGTATTACCGCCTTTGAGTGAGCTGATACCGCTCGCCGCAGCC |
| | GAACGACCGAGCGCAGCGAGTCAGTGAGCGAGGAAGCGGAAGA |
| | GCGCCCAATACGCAAACCGCCTCTCCCCGCGCGTTGGCCGATTC |
| | ATTAATGCAGCAAGCTCATGGCTGACTAATTTTTTTTATTTATGC |
| | AGAGGCCGAGGCCGCCTCGGCCTCTGAGCTATTCCAGAAGTAGT |
| | GAGGAGGCTTTTTTGGAGGCCTAGGCTTTTGCAAAAAGCTCCCC |
| | GTGGCACGACAGGTTTCCCGACTGGAAAGCGGGCAGTGAGCGC |
| | AACGCAATTAATGTGAGTTAGCTCACTCATTAGGCACCCCAGGC |
| | TTTACACTTTATGCTTCCGGCTCGTATGTTGTGTGGAATTGTGAG |
| | CGGATAACAATTTCACACAGGAAACAGCTATGACATGATTACGA |
| | ATTTCACAAATAAAGCATTTTTTTCACTGCATTCTAGTTGTGGTT |
| | TGTCCAAACTCATCAATGTATCTTATCATGTCTGGATCAACTGGA |
| | TAACTCAAGCTAACCAAAATCATCCCAAACTTCCCACCCCATAC |
| | CCTATTACCACTGCCAATTACCTGTGGTTTCATTTACTCTAAACC |
| | TGTGATTCCTCTGAATTATTTTCATTTTAAAGAAATTGTATTTGTT |
| | AAATATGTACTACAAACTtagtagt (SEQ ID NO: 51) |
| Representative | GAATTCGTCGACGCTAGCTGGCTTGTTGTCCACAACCATTAAAC |
| Vector (the | CTTAAAAGCTTTAAAAGCCTTATATATTCTTTTTTTTCTTATAAA |
| TCR- | ACTTAAAACCTTAGAGGCTATTTAAGTTGCTGATTTATATTAATT |
| encoding | TTATTGTTCAAACATGAGAGCTTAGTACGTGAAACATGAGAGCT |
| protein of | TAGTACATTAGCCATGAGAGCTTAGTACATTAGCCATGAGGGTT |
| which can be | TAGTTCATTAAACATGAGAGCTTAGTACATTAAACATGAGAGCT |
| interchanged | TAGTACATACTATCAACAGGTTGAACTGCTGATCTGTACAGTAG |
| with any TCR | AATTGGTAAAGAGAGTTGTGTAAAATATTGAGTTCGCACATCTT |
| sequence of | GTTGTCTGATTATTGATTTTTGGCGAAACCATTTGATCATATGAC |
| interest): | AAGATGTGTATCTACCTTAACTTAATGATTTTGATAAAAATCATT |
| | AGGTACCAATTACATTGCTTGCAATTAACCCTTTAACGGTTATAA |
| | GGATCTAGATGAGATAGAAAGATTTGGTTTTCGGATTTGTGTTA |
| | CATAAGATGCCTAAAATAAAAATTGAGATTCAATTTTTTTTAAA |
| | CTTTTTTTTAATTGGTGGTAAGAATATTCCCTCTACCTGTTTGAG |
| | AGTAATGAAATTGTAGTATGATTTTTCAACAAACTAAAAAAACA |
| | ACATAAATCTCACATAATAACTTTATTTCAATCACACAATTGAAT |
| | ACCAATAGGTTGACAGTACTTACCAGCCTGCAGGTGAAAGACC |
| | CCACCTGTAGGTTTGGCAAGTTAGCTTAAGTAACGCCATTTT |
| | GCAAGGCATGGAAAATACATAACTGAGAATAGAGAAGTTCA |
| | GATCAAGGTTAGGAACAGAGAGACAGCAGAATATGGGCCAA |
| | ACAGGATATCTGTGGTAAGCAGTTCCTGCCCCGGCTCAGGG |
| | CCAAGAACAGATGGTCCCCAGATGCGGTCCCGCCCTCAGCA |
| | GTTTCTAGCGAACCATCAGATGTTTCCAGGGTGCCCCAAGG |
| | ACCTGAAATGACCCTGTGCCTTATTTGAACTAACCAATCAGT |
| | TTGCTTCTTGCTTCTGTTTGTGTGCTTCTGCTCCCTGAGCTC |
| | AATAAAAGAGCCCACAACCCCTCACTTGGTGGGCCAGTCCT |
| | CTGATAGACTGTGTCCCCTGGATACCCGTACGGTACCGCTAGC |
| | GCCACCATGGATACCTGGCTCGTGTGTTGGGCCATCTTTAGCCTG |
| | CTGAAGGCCGGACTGACCGAGCCTGAAGTGACCCAGACTCCAAG |
| | CCATCAAGTGACTCAGATGGGGCAAGAAGTCATTCTGCGTTGCGT |
| | GCCCATCAGCAACCACCTGTACTTTTATTGGTATCGCCAGATCCT |
| | GGGCCAGAAAGTGGAATTCCTGGTGTCCTTCTACAACAATGAGAT |
| | CTCCGAGAAGTCCGAGATCTTCGACGACCAGTTCTCCGTGGAAAG |
| | ACCCGACGGCAGCAACTTCACACTGAAGATCCGGTCTACCAAACT |
| | TGAGGACTCCGCTATGTATTTTTGTGCAATCACAGGTCGCGTTTC |
| | ATATGAGCAATATTTCGGGCCGGGCACCAGGCTCACGGTCACAGA |
| | AGATCTGAACAAGGTGTTCCCTCCAGAGGTGGCCGTGTTCGAGCC |
| | TTCTAAGGCCGAGATCGCCCACACACAAAAAGCCACCCTCGTGTG |
| | CCTGGCCACCGGCTTTTTCCCCGACCACGTGGAACTGTCTTGGTG |
| | GGTCAACGGCAAAGAGGTGCACTCCGGCGTGTCAACGGATCCCC |
| | AGCCTCTGAAAGAACAGCCTGCCCTGAACGACAGCCGGTACTGCC |
| | TGAGCTCCAGACTGAGAGTGTCCGCCACCTTCTGGCAGAACCCCC |
| | GGAACCACTTCAGATGCCAGGTGCAGTTTTACGGCCTGAGCGAGA |
| | ACGACGAGTGGACCCAGGACAGAGCCAAGCCCGTGACACAAATC |
| | GTGTCTGCCGAAGCCTGGGGAAGAGCCGATTGCGGCATCACCAG |
| | CGCCTCCTATCACCAGGGCGTGCTGAGCGCCACAATCCTGTACGA |
| | AATCCTGCTGGGCAAGGCCACCCTGTACGCCGTGCTGGTGTCTGC |
| | TCTGGTGCTGATGGCCATGGTCAAGCGGAAGGACTTTGGCAGCG |
| | GCAGAGCCAAAAGGTCCGGGAGCGGTGCGACAAACTTTAGCCT |
| | GTTGAAACAAGCCGGCGACGTTGAAGAGAACCCCGGACCTATG |
| | CTGCTGATCACCTCCATGCTGGTGCTGTGGATGCAGCTGAG |
| | CCAAGTGAACGGCCAGCAAGTGATGCAGATCCCTCAGTACC |
| | AGCACGTGCAAGAAGGCGAGGACTTCACCACCTACTGCAAC |
| | AGCAGCACCACACTGAGCAACATCCAGTGGTACAAGCAGCG |
| | GCCTGGCGGACACCCTGTGTTTCTGATCCAGCTGGTCAAGT |
| | CCGGCGAAGTGAAGAAGCAGAAGCGGCTGACCTTCCAGTTC |
| | GGCGAGGCCAAGAAGAACAGCAGCCTGCACATCACCGCCAC |
| | ACAGACCACAGATGTGGGCACCTACTTCTGCGCTGGCATCG |
| | GTAGCAGCAACACCGGTAAGCTCATCTTTGGGCAAGGGACA |
| | ACTTTACAAGTAAAACCAGACATCCAGAACCCCGACCCCGCC |
| | GTGTACCAGCTGAGGGACTCCAAGTCCAGCGACAAGAGCGT |
| | GTGTCTGTTTACGGACTTCGACAGCCAGACCAACGTGAGTC |
| | AAAGCAAGGACAGCGACGTCTACATAACGGATAAGACCGTG |
| | CTGGACATGCGGAGCATGGACTTCAAGAGCAACAGCGCCGT |
| | GGCCTGGTCCAACAAGAGCGACTTCGCCTGCGCCAACGCCT |
| | TCAACAACAGCATCATCCCCGAGGACACCTTCTTCCCCAGCA |
| | GCGACGTGCCCTGCGACGTGAAACTGGTGGAGAAGTCCTTC |
| | GAGACAGACACCAATCTGAACTTTCAGAACCTGCTGGTGATC |
| | GTGCTGCGGATTCTGCTGCTGAAAGTGGCCGGCTTCAATCT |
| | GCTGATGACCCTGCGGCTGTGGAGCAGCAGGGCTAAGAGGTC |
| | CGGCAGCGGAGCCACCAATTTTTCCCTGCTGAAACAGGCTGGTG |
| | ACGTGGAAGAAAACCCTGGCCCCATGGCGCTGCCCGTCACCGCG |
| | CTGCTGCTGCCCCTGGCGCTGCTGTTACACGCCGCTCGGCCAGAGC |
| | TTCCCACCCAGGGCACATTCTCCAACGTGTCCACCAATGTGTCGGGA |
| | GGCGGCGGATCGTCCCAGTTCAGAGTGTCCCCTCTGGACCGCACCT |
| | GGAACCTGGGCGAGACCGTGGAGCTGAAATGTCAGGTCCTGCTGAG |
| | CAACCCGACCTCCGGGTGCAGTTGGCTGTTCCAGCCGCGTGGTGCT |
| | GCCGCAAGCCCTACGTTCCTGCTTTACCTGAGCCAGAACAAGCCCAA |
| | GGCGGCCGAGGGCCTGGACACCCAGAGATTCTCCGGCAAGCGCCT |
| | GGGGGACACATTCGTGCTTACTTTGAGCGATTTCCGCAGAGAGAAC |
| | GAGGGCTACTATTTCTGTTCGGCGCTGAGCAATTCCATCATGTATTTC |
| | AGCCACTTTGTGCCAGTGTTCCTGCCTGCCAAGCCTACCACAACACC |
| | AGCTCCCCGTCCCCCGACTCCGGCGCCTACCATCGCGAGTCAACCG |
| | TTGAGCCTGAGGCCTGAGGCTTGTCGGCCCGCTGCGGGGGGTGCC |
| | GTCCACACCAGGGGCCTCGACTTTGCGTGCGACATCTATATTTGGGC |
| | GCCTCTGGCGGGTACCTGCGGGGTGCTGCTGCTGTCATTGGTGATT |
| | ACCCTGTACTGCAATCACCGCAACCGCCGGCGGGTCTGTAAGTGCC |
| | CACGGCCTGTGGTCAAGTCCGGTGACAAACCGTCGCTCTCGGCTCG |
| | CTACGTGCGCGCTAAGCGCAGCGGTTCCGGGGCCACCAACTTTT |
| | CATTGCTGAAGCAGGCCGGTGATGTGGAGGAGAATCCAGGGCC |
| | CATGCGCCCCAGGCTTTGGCTCCTTCTTGCTGCTCAGCTCACTGT |
| | CTTGCATGGCAACTCCGTTCTGCAGCAGACTCCCGCCTACATCA |
| | AGGTGCAGACGAACAAGATGGTGATGCTGTCATGCGAGGCCAA |
| | GATCTCTCTTTCAAATATGAGAATTTATTGGCTACGACAGCGCC |
| | AGGCCCCCTCCAGCGACAGCCACCACGAGTTCCTGGCGCTTTGG |
| | GATTCTGCTAAAGGCACCATCCATGGAGAGGAGGTGGAACAGG |
| | AGAAGATAGCTGTCTTCCGCGACGCATCCCGCTTCATCCTGAAC |
| | CTGACCAGCGTGAAGCCGGAGGACAGCGGCATCTACTTCTGTAT |
| | GATCGTTGGCTCCCCCGAGCTGACCTTCGGCAAAGGCACCCAGC |
| | TGTCCGTGGTGGACTTCCTGCCCACCACAGCCCAGCCAACCAAG |
| | AAATCCACCCTCAAGAAGCGCGTGTGCCGACTGCCCCGCCCTGA |
| | AACCCAGAAGGGCCCTCTGTGCTCCCCCATCACCCTTGGACTGC |
| | TGGTGGCGGGAGTCCTGGTGCTGCTCGTATCTCTGGGTGTCGCC |
| | ATCCACCTGTGCTGCCGCCGCCGCCGCGCCCGCCTGAGGTTTAT |
| | GAAACAGTTTTACAAGTGATAAATCGATGGAAGGGTGGCATCCC |
| | TGTGACCCCTCCCCAGTGCCTCTCCTGGCCCTGGAAGTTGCCACT |
| | CCAGTGCCCACCAGCCTTGTCCTAATAAAATTAAGTTGCATCATT |
| | TTGTCTGACTAGGTGTCCTTCTATAATATTATGGGGTGGAGGGG |
| | GGTGGTATGGAGCAAGGGGCAAGTTGGGAAGACAACCTGTAGG |
| | GCCTGCGGGGTCTATTGGGAACCAAGCTGGAGTGCAGTGGCACA |
| | ATCTTGGCTCACTGCAATCTCCGCCTCCTGGGTTCAAGCGATTCT |
| | CCTGCCTCAGCCTCCCGAGTTGTTGGGATTCCAGGCATGCATGA |
| | CCAGGCTCAGCTAATTTTTGTTTTTTTGGTAGAGACGGGGTTTCA |
| | CCATATTGGCCAGGCTGGTCTCCAACTCCTAATCTCAGGTGATCT |
| | ACCCACCTTGGCCTCCCAAATTGCTGGGATTACAGGCGTGAACC |
| | ACTGCTCCCTTCCCTGTCCTTCTGATTACTAGTGGCTCCGGTGCC |
| | CGTCAGTGGGCAGAGCGCACATCGCCCACAGTCCCCGAGAAGTT |
| | GTGGGGAGGGGTCGGCAATTGAACCGGTGCCTAGAGAAGGTGG |
| | CGCGGGGTAAACTGGGAAAGTGATGTCGTGTACTGGCTCCGCCT |
| | TTTTCCCGAGGGTGGGGGAGAACCGTATATAAGTGCAGTAGTCG |
| | CCGTGAACGTTCTTTTTCGCAACGGGTTTGCCGCCAGAACACAG |
| | GTAAGTGCCGTGTGTGGTTCCCGCGGGCCTGGCCTCTTTACGGG |
| | TTATGGCCCTTGCGTGCCTTGAATTACTTCCACCTGGCTGCAGTA |
| | CGTGATTCTTGATCCCGAGCTTCGGGTTGGAAGTGGGTGGGAGA |
| | GTTCGAGGCCTTGCGCTTAAGGAGCCCCTTCGCCTCGTGCTTGA |
| | GTTGAGGCCTGGCCTGGGCGCTGGGGCCGCCGCGTGCGAATCTG |
| | GTGGCACCTTCGCGCCTGTCTCGCTGCTTTCGATAAGTCTCTAGC |
| | CATTTAAAATTTTTGATGACCTGCTGCGACGCTTTTTTTCTGGCA |
| | AGATAGTCTTGTAAATGCGGGCCAAGATCTGCACACTGGTATTT |
| | CGGTTTTTGGGGCCGCGGGCGGCGACGGGGCCCGTGCGTCCCAG |
| | CGCACATGTTCGGCGAGGCGGGGCCTGCGAGCGCGGCCACCGA |
| | GAATCGGACGGGGGTAGTCTCAAGCTGGCCGGCCTGCTCTGGTG |
| | CCTGGCCTCGCGCCGCCGTGTATCGCCCCGCCCTGGGCGGCAAG |
| | GCTGGCCCGGTCGGCACCAGTTGCGTGAGCGGAAAGATGGCCG |
| | CTTCCCGGCCCTGCTGCAGGGAGCTCAAAATGGAGGACGCGGCG |
| | CTCGGGAGAGCGGGGGGTGAGTCACCCACACAAAGGAAAAGG |
| | GCCTTTCCGTCCTCAGCCGTCGCTTCATGTGACTCCACGGAGTAC |
| | CGGGCGCCGTCCAGGCACCTCGATTAGTTCTCGAGCTTTTGGAG |
| | TACGTCGTCTTTAGGTTGGGGGGAGGGGTTTTATGCGATGGAGT |
| | TTCCCCACACTGAGTGGGTGGAGACTGAAGTTAGGCCAGCTTGG |
| | CACTTGATGTAATTCTCCTTGGAATTTGCCCTTTTTGAGTTTGGA |
| | TCTTGGTTCATTCTCAAGCCTCAGACAGTGGTTCAAAGTTTTTTT |
| | CTTCCATTTCAGGTGTCGTGAACTAGTCCAGTGTGGTGGAATTCT |
| | GCAGATATCACGGCTAGCGCCACCATGGGTCGGGGGCTGCTCAG |
| | GGGCCTGTGGCCGCTGCACATCGTCCTGTGGACGCGTATCGCCA |
| | GCACGATCCCACCGCACGTTCAGAAGTCGGTGAATAACGACATG |
| | ATAGTCACTGACAACAACGGTGCAGTCAAGTTTCCACAACTGTG |
| | TAAATTTTGTGATGTGAGATTTTCCACCTGTGACAACCAGAAAT |
| | CCTGCATGAGCAACTGCAGCATCACCTCCATCTGTGAGAAGCCA |
| | CAGGAAGTCTGTGTGGCTGTATGGAGAAAGAATGACGAGAACA |
| | TAACACTAGAGACAGTTTGCCATGACCCCAAGCTCCCCTACCAT |
| | GACTTTATTCTGGAAGATGCTGCTTCTCCAAAGTGCATTATGAA |
| | GGAGAAGAAAAAGCCTGGTGAGACTTTCTTCATGTGTTCCTGTA |
| | GCTCTGATGAGTGCAATGACAACATCATCTTCTCAGAAGAATAT |
| | AACACCAGCAATCCTGACTTGTTGCTAGTCATATTTCAAGTGAC |
| | AGGCATCAGCCTCCTGCCACCACTGGGAGTTGCCATATCTGTCA |
| | TCATCATCTTCTACTGCTACCGCGTGAACCGGCAGCAGAAGGCT |
| | AGTGGTTCAGGCGCAACGAATTTCTCTTTGCTGAAGCAGGCTGG |
| | GGATGTCGAAGAAAATCCGGGTCCAATGGTGGGCTCGCTCAACT |
| | GCATCGTAGCAGTCTCCCAGAATATGGGCATCGGGAAGAACGGT |
| | GATTTCCCGTGGCCCCCACTTCGCAACGAGAGCCGTTATTTCCA |
| | AAGAATGACTACAACCTCCTCCGTGGAGGGTAAGCAGAACCTG |
| | GTCATCATGGGGAAGAAGACCTGGTTCTCTATCCCTGAAAAAAA |
| | CCGCCCCCTGAAGGGCCGCATCAACCTGGTGCTGAGCAGGGAAC |
| | TCAAGGAGCCTCCTCAGGGCGCGCATTTTCTGAGCCGCTCATTG |
| | GATGACGCTCTCAAACTGACCGAACAGCCGGAGCTAGCCAACA |
| | AGGTGGACATGGTGTGGATCGTCGGAGGCTCCTCCGTGTACAAG |
| | GAGGCCATGAATCACCCCGGCCACTTGAAGCTGTTCGTCACCCG |
| | GATCATGCAGGACTTCGAGTCGGACACGTTCTTTCCAGAGATTG |
| | ACCTGGAGAAGTACAAGCTGCTGCCCGAGTACCCGGGAGTTCTT |
| | AGTGATGTGCAGGAGGAGAAAGGCATCAAGTACAAATTTGAGG |
| | TGTACGAGAAGAACGACTAACGGTCCGTCCTGACCAATGCTGGA |
| | GTTCTTCGCCCACCCCAACTTGTTTATTGCAGCTTATAATGGTTA |
| | CAAATAAAGCAATAGCATCACAAATTTCACAAATAAAGCATTTT |
| | TTTCACTGCATTCTAGTTGTGGTTTGTCCAAACTCATCAATGTAT |
| | CTTATCATGTCTGTATACAGGTTACCTCAGTCTCCTAGGTACGTC |
| | TTATATCTATGAAAAAACATTCAAAAGCACAACATCTAGAAGAA |
| | CTTACCTTTTTTCACCACTCTATTGCAAAGATATGTACCGATTTC |
| | TCTCGAAGTACAAAAAACCGCTAGTTTTCAAATTCACCTCAAGA |
| | CTTTGAAAAAAAATTGAATCTGTCAATGTCAAATAAAATCAGAA |
| | ACAAATGTCATAATGTTACGTTAATGTTGTCAGGTCGAAAAATA |
| | AAATTGCAAATAGAAATTTTGTTCCTTTTTTATTGGTTTTTATTG |
| | GTGGGAAAAATATTCCCTCTAACTGCAAAAGGGTTAATTATGTT |
| | AGAGGTAGAGTCGACAAGCTT (SEQ ID NO: 50) |
| | Map of the pNVVD154_TSC-200-A02_TCR-28_MSCV-TCR28-CD8- |
| | EF1a-TGFR-DHFR Vector |
| | GAATTCGTCGACGCTAGCTGGCTTGTTGTCCACAACCATTAAAC |
| | CTTAAAAGCTTTAAAAGCCTTATATATTCTTTTTTTTCTTATAAA |
| | ACTTAAAACCTTAGAGGCTATTTAAGTTGCTGATTTATATTAATT |
| | TTATTGTTCAAACATGAGAGCTTAGTACGTGAAACATGAGAGCT |
| | TAGTACATTAGCCATGAGAGCTTAGTACATTAGCCATGAGGGTT |
| | TAGTTCATTAAACATGAGAGCTTAGTACATTAAACATGAGAGCT |
| | TAGTACATACTATCAACAGGTTGAACTGCTGATCTGTACAGTAG |
| | AATTGGTAAAGAGAGTTGTGTAAAATATTGAGTTCGCACATCTT |
| | GTTGTCTGATTATTGATTTTTGGCGAAACCATTTGATCATATGAC |
| | AAGATGTGTATCTACCTTAACTTAATGATTTTGATAAAAATCATT |
| | AGGTACCAATTACATTGCTTGCAATTAACCCTTTAACGGTTATAA |
| | GGATCTAGATGAGATAGAAAGATTTGGTTTTCGGATTTGTGTTA |
| | CATAAGATGCCTAAAATAAAAATTGAGATTCAATTTTTTTTAAA |
| | CTTTTTTTTAATTGGTGGTAAGAATATTCCCTCTACCTGTTTGAG |
| | AGTAATGAAATTGTAGTATGATTTTTCAACAAACTAAAAAAACA |
| | ACATAAATCTCACATAATAACTTTATTTCAATCACACAATTGAAT |
| | ACCAATAGGTTGACAGTACTTACCAGCCTGCAGGTGAAAGACC |
| | CCACCTGTAGGTTTGGCAAGTTAGCTTAAGTAACGCCATTTT |
| | GCAAGGCATGGAAAATACATAACTGAGAATAGAGAAGTTCA |
| | GATCAAGGTTAGGAACAGAGAGACAGCAGAATATGGGCCAA |
| | ACAGGATATCTGTGGTAAGCAGTTCCTGCCCCGGCTCAGGG |
| | CCAAGAACAGATGGTCCCCAGATGCGGTCCCGCCCTCAGCA |
| | GTTTCTAGCGAACCATCAGATGTTTCCAGGGTGCCCCAAGG |
| | ACCTGAAATGACCCTGTGCCTTATTTGAACTAACCAATCAGT |
| | TTGCTTCTTGCTTCTGTTTGTGTGCTTCTGCTCCCTGAGCTC |
| | AATAAAAGAGCCCACAACCCCTCACTTGGTGGGCCAGTCCT |
| | CTGATAGACTGTGTCCCCTGGATACCCGTACGGTACCGCTAGC |
| | GCCACCATGGATACCTGGCTCGTGTGTTGGGCCATCTTTAGCCTG |
| | CTGAAGGCCGGACTGACCGAGCCTGAAGTGACCCAGACTCCAAG |
| | CCATCAAGTGACTCAGATGGGGCAAGAAGTCATTCTGCGTTGCGT |
| | GCCCATCAGCAACCACCTGTACTTTTATTGGTATCGCCAGATCCT |
| | GGGCCAGAAAGTGGAATTCCTGGTGTCCTTCTACAACAATGAGAT |
| | CTCCGAGAAGTCCGAGATCTTCGACGACCAGTTCTCCGTGGAAAG |
| | ACCCGACGGCAGCAACTTCACACTGAAGATCCGGTCTACCAAACT |
| | TGAGGACTCCGCTATGTATTTTTGTGCAATCACAGGTCGCGTTTC |
| | ATATGAGCAATATTTCGGGCCGGGCACCAGGCTCACGGTCACAGA |
| | AGATCTGAACAAGGTGTTCCCTCCAGAGGTGGCCGTGTTCGAGCC |
| | TTCTAAGGCCGAGATCGCCCACACACAAAAAGCCACCCTCGTGTG |
| | CCTGGCCACCGGCTTTTTCCCCGACCACGTGGAACTGTCTTGGTG |
| | GGTCAACGGCAAAGAGGTGCACTCCGGCGTGTCAACGGATCCCC |
| | AGCCTCTGAAAGAACAGCCTGCCCTGAACGACAGCCGGTACTGCC |
| | TGAGCTCCAGACTGAGAGTGTCCGCCACCTTCTGGCAGAACCCCC |
| | GGAACCACTTCAGATGCCAGGTGCAGTTTTACGGCCTGAGCGAGA |
| | ACGACGAGTGGACCCAGGACAGAGCCAAGCCCGTGACACAAATC |
| | GTGTCTGCCGAAGCCTGGGGAAGAGCCGATTGCGGCATCACCAG |
| | CGCCTCCTATCACCAGGGCGTGCTGAGCGCCACAATCCTGTACGA |
| | AATCCTGCTGGGCAAGGCCACCCTGTACGCCGTGCTGGTGTCTGC |
| | TCTGGTGCTGATGGCCATGGTCAAGCGGAAGGACTTTGGCAGCG |
| | GCAGAGCCAAAAGGTCCGGGAGCGGTGCGACAAACTTTAGCCT |
| | GTTGAAACAAGCCGGCGACGTTGAAGAGAACCCCGGACCTATG |
| | CTGCTGATCACCTCCATGCTGGTGCTGTGGATGCAGCTGAG |
| | CCAAGTGAACGGCCAGCAAGTGATGCAGATCCCTCAGTACC |
| | AGCACGTGCAAGAAGGCGAGGACTTCACCACCTACTGCAAC |
| | AGCAGCACCACACTGAGCAACATCCAGTGGTACAAGCAGCG |
| | GCCTGGCGGACACCCTGTGTTTCTGATCCAGCTGGTCAAGT |
| | CCGGCGAAGTGAAGAAGCAGAAGCGGCTGACCTTCCAGTTC |
| | GGCGAGGCCAAGAAGAACAGCAGCCTGCACATCACCGCCAC |
| | ACAGACCACAGATGTGGGCACCTACTTCTGCGCTGGCATCG |
| | GTAGCAGCAACACCGGTAAGCTCATCTTTGGGCAAGGGACA |
| | ACTTTACAAGTAAAACCAGACATCCAGAACCCCGACCCCGCC |
| | GTGTACCAGCTGAGGGACTCCAAGTCCAGCGACAAGAGCGT |
| | GTGTCTGTTTACGGACTTCGACAGCCAGACCAACGTGAGTC |
| | AAAGCAAGGACAGCGACGTCTACATAACGGATAAGACCGTG |
| | CTGGACATGCGGAGCATGGACTTCAAGAGCAACAGCGCCGT |
| | GGCCTGGTCCAACAAGAGCGACTTCGCCTGCGCCAACGCCT |
| | TCAACAACAGCATCATCCCCGAGGACACCTTCTTCCCCAGCA |
| | GCGACGTGCCCTGCGACGTGAAACTGGTGGAGAAGTCCTTC |
| | GAGACAGACACCAATCTGAACTTTCAGAACCTGCTGGTGATC |
| | GTGCTGCGGATTCTGCTGCTGAAAGTGGCCGGCTTCAATCT |
| | GCTGATGACCCTGCGGCTGTGGAGCAGCAGGGCTAAGAGGTC |
| | CGGCAGCGGAGCCACCAATTTTTCCCTGCTGAAACAGGCTGGTG |
| | ACGTGGAAGAAAACCCTGGCCCCATGGCGCTGCCCGTCACCGCG |
| | CTGCTGCTGCCCCTGGCGCTGCTGTTACACGCCGCTCGGCCAGAGC |
| | TTCCCACCCAGGGCACATTCTCCAACGTGTCCACCAATGTGTCGGGA |
| | GGCGGCGGATCGTCCCAGTTCAGAGTGTCCCCTCTGGACCGCACCT |
| | GGAACCTGGGCGAGACCGTGGAGCTGAAATGTCAGGTCCTGCTGAG |
| | CAACCCGACCTCCGGGTGCAGTTGGCTGTTCCAGCCGCGTGGTGCT |
| | GCCGCAAGCCCTACGTTCCTGCTTTACCTGAGCCAGAACAAGCCCAA |
| | GGCGGCCGAGGGCCTGGACACCCAGAGATTCTCCGGCAAGCGCCT |
| | GGGGGACACATTCGTGCTTACTTTGAGCGATTTCCGCAGAGAGAAC |
| | GAGGGCTACTATTTCTGTTCGGCGCTGAGCAATTCCATCATGTATTTC |
| | AGCCACTTTGTGCCAGTGTTCCTGCCTGCCAAGCCTACCACAACACC |
| | AGCTCCCCGTCCCCCGACTCCGGCGCCTACCATCGCGAGTCAACCG |
| | TTGAGCCTGAGGCCTGAGGCTTGTCGGCCCGCTGCGGGGGGTGCC |
| | GTCCACACCAGGGGCCTCGACTTTGCGTGCGACATCTATATTTGGGC |
| | GCCTCTGGCGGGTACCTGCGGGGTGCTGCTGCTGTCATTGGTGATT |
| | ACCCTGTACTGCAATCACCGCAACCGCCGGCGGGTCTGTAAGTGCC |
| | CACGGCCTGTGGTCAAGTCCGGTGACAAACCGTCGCTCTCGGCTCG |
| | CTACGTGCGCGCTAAGCGCAGCGGTTCCGGGGCCACCAACTTTT |
| | CATTGCTGAAGCAGGCCGGTGATGTGGAGGAGAATCCAGGGCC |
| | CATGCGCCCCAGGCTTTGGCTCCTTCTTGCTGCTCAGCTCACTGT |
| | CTTGCATGGCAACTCCGTTCTGCAGCAGACTCCCGCCTACATCA |
| | AGGTGCAGACGAACAAGATGGTGATGCTGTCATGCGAGGCCAA |
| | GATCTCTCTTTCAAATATGAGAATTTATTGGCTACGACAGCGCC |
| | AGGCCCCCTCCAGCGACAGCCACCACGAGTTCCTGGCGCTTTGG |
| | GATTCTGCTAAAGGCACCATCCATGGAGAGGAGGTGGAACAGG |
| | AGAAGATAGCTGTCTTCCGCGACGCATCCCGCTTCATCCTGAAC |
| | CTGACCAGCGTGAAGCCGGAGGACAGCGGCATCTACTTCTGTAT |
| | GATCGTTGGCTCCCCCGAGCTGACCTTCGGCAAAGGCACCCAGC |
| | TGTCCGTGGTGGACTTCCTGCCCACCACAGCCCAGCCAACCAAG |
| | AAATCCACCCTCAAGAAGCGCGTGTGCCGACTGCCCCGCCCTGA |
| | AACCCAGAAGGGCCCTCTGTGCTCCCCCATCACCCTTGGACTGC |
| | TGGTGGCGGGAGTCCTGGTGCTGCTCGTATCTCTGGGTGTCGCC |
| | ATCCACCTGTGCTGCCGCCGCCGCCGCGCCCGCCTGAGGTTTAT |
| | GAAACAGTTTTACAAGTGATAAATCGATGGAAGGGTGGCATCCC |
| | TGTGACCCCTCCCCAGTGCCTCTCCTGGCCCTGGAAGTTGCCACT |
| | CCAGTGCCCACCAGCCTTGTCCTAATAAAATTAAGTTGCATCATT |
| | TTGTCTGACTAGGTGTCCTTCTATAATATTATGGGGTGGAGGGG |
| | GGTGGTATGGAGCAAGGGGCAAGTTGGGAAGACAACCTGTAGG |
| | GCCTGCGGGGTCTATTGGGAACCAAGCTGGAGTGCAGTGGCACA |
| | ATCTTGGCTCACTGCAATCTCCGCCTCCTGGGTTCAAGCGATTCT |
| | CCTGCCTCAGCCTCCCGAGTTGTTGGGATTCCAGGCATGCATGA |
| | CCAGGCTCAGCTAATTTTTGTTTTTTTGGTAGAGACGGGGTTTCA |
| | CCATATTGGCCAGGCTGGTCTCCAACTCCTAATCTCAGGTGATCT |
| | ACCCACCTTGGCCTCCCAAATTGCTGGGATTACAGGCGTGAACC |
| | ACTGCTCCCTTCCCTGTCCTTCTGATTACTAGTGGCTCCGGTGCC |
| | CGTCAGTGGGCAGAGCGCACATCGCCCACAGTCCCCGAGAAGTT |
| | GTGGGGAGGGGTCGGCAATTGAACCGGTGCCTAGAGAAGGTGG |
| | CGCGGGGTAAACTGGGAAAGTGATGTCGTGTACTGGCTCCGCCT |
| | TTTTCCCGAGGGTGGGGGAGAACCGTATATAAGTGCAGTAGTCG |
| | CCGTGAACGTTCTTTTTCGCAACGGGTTTGCCGCCAGAACACAG |
| | GTAAGTGCCGTGTGTGGTTCCCGCGGGCCTGGCCTCTTTACGGG |
| | TTATGGCCCTTGCGTGCCTTGAATTACTTCCACCTGGCTGCAGTA |
| | CGTGATTCTTGATCCCGAGCTTCGGGTTGGAAGTGGGTGGGAGA |
| | GTTCGAGGCCTTGCGCTTAAGGAGCCCCTTCGCCTCGTGCTTGA |
| | GTTGAGGCCTGGCCTGGGCGCTGGGGCCGCCGCGTGCGAATCTG |
| | GTGGCACCTTCGCGCCTGTCTCGCTGCTTTCGATAAGTCTCTAGC |
| | CATTTAAAATTTTTGATGACCTGCTGCGACGCTTTTTTTCTGGCA |
| | AGATAGTCTTGTAAATGCGGGCCAAGATCTGCACACTGGTATTT |
| | CGGTTTTTGGGGCCGCGGGCGGCGACGGGGCCCGTGCGTCCCAG |
| | CGCACATGTTCGGCGAGGCGGGGCCTGCGAGCGCGGCCACCGA |
| | GAATCGGACGGGGGTAGTCTCAAGCTGGCCGGCCTGCTCTGGTG |
| | CCTGGCCTCGCGCCGCCGTGTATCGCCCCGCCCTGGGCGGCAAG |
| | GCTGGCCCGGTCGGCACCAGTTGCGTGAGCGGAAAGATGGCCG |
| | CTTCCCGGCCCTGCTGCAGGGAGCTCAAAATGGAGGACGCGGCG |
| | CTCGGGAGAGCGGGCGGGTGAGTCACCCACACAAAGGAAAAGG |
| | GCCTTTCCGTCCTCAGCCGTCGCTTCATGTGACTCCACGGAGTAC |
| | CGGGCGCCGTCCAGGCACCTCGATTAGTTCTCGAGCTTTTGGAG |
| | TACGTCGTCTTTAGGTTGGGGGGAGGGGTTTTATGCGATGGAGT |
| | TTCCCCACACTGAGTGGGTGGAGACTGAAGTTAGGCCAGCTTGG |
| | CACTTGATGTAATTCTCCTTGGAATTTGCCCTTTTTGAGTTTGGA |
| | TCTTGGTTCATTCTCAAGCCTCAGACAGTGGTTCAAAGTTTTTTT |
| | CTTCCATTTCAGGTGTCGTGAACTAGTCCAGTGTGGTGGAATTCT |
| | GCAGATATCACGGCTAGCGCCACCATGGGTCGGGGGCTGCTCAG |
| | GGGCCTGTGGCCGCTGCACATCGTCCTGTGGACGCGTATCGCCA |
| | GCACGATCCCACCGCACGTTCAGAAGTCGGTGAATAACGACATG |
| | ATAGTCACTGACAACAACGGTGCAGTCAAGTTTCCACAACTGTG |
| | TAAATTTTGTGATGTGAGATTTTCCACCTGTGACAACCAGAAAT |
| | CCTGCATGAGCAACTGCAGCATCACCTCCATCTGTGAGAAGCCA |
| | CAGGAAGTCTGTGTGGCTGTATGGAGAAAGAATGACGAGAACA |
| | TAACACTAGAGACAGTTTGCCATGACCCCAAGCTCCCCTACCAT |
| | GACTTTATTCTGGAAGATGCTGCTTCTCCAAAGTGCATTATGAA |
| | GGAGAAGAAAAAGCCTGGTGAGACTTTCTTCATGTGTTCCTGTA |
| | GCTCTGATGAGTGCAATGACAACATCATCTTCTCAGAAGAATAT |
| | AACACCAGCAATCCTGACTTGTTGCTAGTCATATTTCAAGTGAC |
| | AGGCATCAGCCTCCTGCCACCACTGGGAGTTGCCATATCTGTCA |
| | TCATCATCTTCTACTGCTACCGCGTGAACCGGCAGCAGAAGGCT |
| | AGTGGTTCAGGCGCAACGAATTTCTCTTTGCTGAAGCAGGCTGG |
| | GGATGTCGAAGAAAATCCGGGTCCAATGGTGGGCTCGCTCAACT |
| | GCATCGTAGCAGTCTCCCAGAATATGGGCATCGGGAAGAACGGT |
| | GATTTCCCGTGGCCCCCACTTCGCAACGAGAGCCGTTATTTCCA |
| | AAGAATGACTACAACCTCCTCCGTGGAGGGTAAGCAGAACCTG |
| | GTCATCATGGGGAAGAAGACCTGGTTCTCTATCCCTGAAAAAAA |
| | CCGCCCCCTGAAGGGCCGCATCAACCTGGTGCTGAGCAGGGAAC |
| | TCAAGGAGCCTCCTCAGGGCGCGCATTTTCTGAGCCGCTCATTG |
| | GATGACGCTCTCAAACTGACCGAACAGCCGGAGCTAGCCAACA |
| | AGGTGGACATGGTGTGGATCGTCGGAGGCTCCTCCGTGTACAAG |
| | GAGGCCATGAATCACCCCGGCCACTTGAAGCTGTTCGTCACCCG |
| | GATCATGCAGGACTTCGAGTCGGACACGTTCTTTCCAGAGATTG |
| | ACCTGGAGAAGTACAAGCTGCTGCCCGAGTACCCGGGAGTTCTT |
| | AGTGATGTGCAGGAGGAGAAAGGCATCAAGTACAAATTTGAGG |
| | TGTACGAGAAGAACGACTAACGGTCCGTCCTGACCAATGCTGGA |
| | GTTCTTCGCCCACCCCAACTTGTTTATTGCAGCTTATAATGGTTA |
| | CAAATAAAGCAATAGCATCACAAATTTCACAAATAAAGCATTTT |
| | TTTCACTGCATTCTAGTTGTGGTTTGTCCAAACTCATCAATGTAT |
| | CTTATCATGTCTGTATACAGGTTACCTCAGTCTCCTAGGTACGTC |
| | TTATATCTATGAAAAAACATTCAAAAGCACAACATCTAGAAGAA |
| | CTTACCTTTTTTCACCACTCTATTGCAAAGATATGTACCGATTTC |
| | TCTCGAAGTACAAAAAACCGCTAGTTTTCAAATTCACCTCAAGA |
| | CTTTGAAAAAAAATTGAATCTGTCAATGTCAAATAAAATCAGAA |
| | ACAAATGTCATAATGTTACGTTAATGTTGTCAGGTCGAAAAATA |
| | AAATTGCAAATAGAAATTTTGTTCCTTTTTTATTGGTTTTTATTG |
| | GTGGGAAAAATATTCCCTCTAACTGCAAAAGGGTTAATTATGTT |
| | AGAGGTAGAGTCGACAAGCTT |
| | [symbol] |
| | Map of the pNVVD154_TSC-200-A02_TCR-28_MSCV-TCR28-CD8- |
| | EF1a-TGFR-DHFR Vector |
| | pNYVD154_TSC-200-A02_TCR-28_MSCV-TCR28-COB-EFia-TGFR-DHFR |
| | Key: CD: cluster of differentiation RNA-OUT: anti-sense RNA against the |
| | bacterial levansucrase encoded by sacB. SV: simian virus TCR: T Cell |
| | Receptor, TIR: terminal inverted repeat, QBend: Mouse anti Human CD34 |
| | antibody, dnTGFbRII: Dominant-negative TGF beta Receptor II, DHFR: |
| | Dihydrofolate reductase selection marker |
| Representative | GAATTCGTCGACGCTAGCTGGCTTGTTGTCCACAACCATTAAAC |
| Vector (the | CTTAAAAGCTTTAAAAGCCTTATATATTCTTTTTTTTCTTATAAA |
| TCR- | ACTTAAAACCTTAGAGGCTATTTAAGTTGCTGATTTATATTAATT |
| encoding | TTATTGTTCAAACATGAGAGCTTAGTACGTGAAACATGAGAGCT |
| protein of | TAGTACATTAGCCATGAGAGCTTAGTACATTAGCCATGAGGGTT |
| which can be | TAGTTCATTAAACATGAGAGCTTAGTACATTAAACATGAGAGCT |
| interchanged | TAGTACATACTATCAACAGGTTGAACTGCTGATCTGTACAGTAG |
| with any TCR | AATTGGTAAAGAGAGTTGTGTAAAATATTGAGTTCGCACATCTT |
| sequence of | GTTGTCTGATTATTGATTTTTGGCGAAACCATTTGATCATATGAC |
| interest): | AAGATGTGTATCTACCTTAACTTAATGATTTTGATAAAAATCATT |
| pNVVD160_T | AGGTACCAATTACATTGCTTGCAATTAACCCTTTAACGGTTATAA |
| SC-200- | GGATCTAGATGAGATAGAAAGATTTGGTTTTCGGATTTGTGTTA |
| A02_TCR- | CATAAGATGCCTAAAATAAAAATTGAGATTCAATTTTTTTTAAA |
| 28_MSCV- | CTTTTTTTTAATTGGTGGTAAGAATATTCCCTCTACCTGTTTGAG |
| TCR28-CD8- | AGTAATGAAATTGTAGTATGATTTTTCAACAAACTAAAAAAACA |
| EF1a-TGFR- | ACATAAATCTCACATAATAACTTTATTTCAATCACACAATTGAAT |
| DHFR | ACCAATAGGTTGACAGTACTTACCAGCCTGCAGGTGAAAGACC |
| | CCACCTGTAGGTTTGGCAAGTTAGCTTAAGTAACGCCATTTT |
| | GCAAGGCATGGAAAATACATAACTGAGAATAGAGAAGTTCA |
| | GATCAAGGTTAGGAACAGAGAGACAGCAGAATATGGGCCAA |
| | ACAGGATATCTGTGGTAAGCAGTTCCTGCCCCGGCTCAGGG |
| | CCAAGAACAGATGGTCCCCAGATGCGGTCCCGCCCTCAGCA |
| | GTTTCTAGCGAACCATCAGATGTTTCCAGGGTGCCCCAAGG |
| | ACCTGAAATGACCCTGTGCCTTATTTGAACTAACCAATCAGT |
| | TTGCTTCTTGCTTCTGTTTGTGTGCTTCTGCTCCCTGAGCTC |
| | AATAAAAGAGCCCACAACCCCTCACTTGGTGGGCCAGTCCT |
| | CTGATAGACTGTGTCCCCTGGATACCCGTACGGTACCGCTAGC |
| | GCCACCATGGATACCTGGCTCGTGTGTTGGGCCATCTTTAGCCTG |
| | CTGAAGGCCGGACTGACCGAGCCTGAAGTGACCCAGACTCCAAG |
| | CCATCAAGTGACTCAGATGGGGCAAGAAGTCATTCTGCGTTGCGT |
| | GCCCATCAGCAACCACCTGTACTTTTATTGGTATCGCCAGATCCT |
| | GGGCCAGAAAGTGGAATTCCTGGTGTCCTTCTACAACAATGAGAT |
| | CTCCGAGAAGTCCGAGATCTTCGACGACCAGTTCTCCGTGGAAAG |
| | ACCCGACGGCAGCAACTTCACACTGAAGATCCGGTCTACCAAACT |
| | TGAGGACTCCGCTATGTATTTTTGTGCAATCACAGGTCGCGTTTC |
| | ATATGAGCAATATTTCGGGCCGGGCACCAGGCTCACGGTCACAGA |
| | AGATCTCAATAAAGTGTTCCCCCCTGAGGTTGCGGTGTTTGAGCC |
| | GTCCAAAGCGGAGATTGCCCACACACAGAAAGCGACTTTGGTTTG |
| | TTTGGCGACAGGCTTTTTCCCTGACCACGTAGAGCTGTCTTGGTG |
| | GGTCAACGGCAAGGAGGTTCACAGCGGTGTGTCAACGGATCCCC |
| | AGCCTCTGAAAGAACAGCCTGCCCTGAACGACAGCCGGTACTGCC |
| | TGAGCTCCAGACTGAGAGTGTCCGCCACCTTCTGGCAGAACCCCC |
| | GGAACCACTTCAGATGCCAGGTGCAGTTTTACGGCCTGAGCGAGA |
| | ACGACGAGTGGACCCAGGACAGAGCCAAGCCCGTGACACAAATC |
| | GTGTCTGCCGAAGCCTGGGGAAGAGCCGATTGCGGCATCACCAG |
| | CGCCTCCTATCACCAGGGCGTGCTGAGCGCCACAATCCTGTACGA |
| | AATCCTGCTGGGCAAGGCCACCCTGTACGCCGTGCTGGTGTCTGC |
| | TCTGGTGCTGATGGCCATGGTCAAGCGGAAGGACTTTGGCAGCG |
| | GCAGAGCCAAAAGGTCCGGGAGCGGTGCGACAAACTTTAGCCT |
| | GTTGAAACAAGCCGGCGACGTTGAAGAGAACCCCGGACCTATG |
| | CTGCTGATCACCTCCATGCTGGTGCTGTGGATGCAGCTGAG |
| | CCAAGTGAACGGCCAGCAAGTGATGCAGATCCCTCAGTACC |
| | AGCACGTGCAAGAAGGCGAGGACTTCACCACCTACTGCAAC |
| | AGCAGCACCACACTGAGCAACATCCAGTGGTACAAGCAGCG |
| | GCCTGGCGGACACCCTGTGTTTCTGATCCAGCTGGTCAAGT |
| | CCGGCGAAGTGAAGAAGCAGAAGCGGCTGACCTTCCAGTTC |
| | GGCGAGGCCAAGAAGAACAGCAGCCTGCACATCACCGCCAC |
| | ACAGACCACAGATGTGGGCACCTACTTCTGCGCTGGCATCG |
| | GTAGCAGCAACACCGGTAAGCTCATCTTTGGGCAAGGGACA |
| | ACTTTACAAGTAAAACCAGACATCCAGAACCCCGACCCCGCC |
| | GTGTACCAGCTGAGGGACTCCAAGTCCAGCGACAAGAGCGT |
| | GTGTCTGTTTACGGACTTCGACAGCCAGACCAACGTGAGTC |
| | AAAGCAAGGACAGCGACGTCTACATAACGGATAAGACCGTG |
| | CTGGACATGCGGAGCATGGACTTCAAGAGCAACAGCGCCGT |
| | GGCCTGGTCCAACAAGAGCGACTTCGCCTGCGCCAACGCCT |
| | TCAACAACAGCATCATCCCCGAGGACACCTTCTTCCCCAGCA |
| | GCGACGTGCCCTGCGACGTGAAACTGGTGGAGAAGTCCTTC |
| | GAGACAGACACCAATCTGAACTTTCAGAACCTGCTGGTGATC |
| | GTGCTGCGGATTCTGCTGCTGAAAGTGGCCGGCTTCAATCT |
| | GCTGATGACCCTGCGGCTGTGGAGCAGCAGGGCTAAGAGGTC |
| | CGGCAGCGGAGCCACCAATTTTTCCCTGCTGAAACAGGCTGGTG |
| | ACGTGGAAGAAAACCCTGGCCCCATGGCGCTGCCCGTCACCGCG |
| | CTGCTGCTGCCCCTGGCGCTGCTGTTACACGCCGCTCGGCCAGAGC |
| | TTCCCACCCAGGGCACATTCTCCAACGTGTCCACCAATGTGTCGGGA |
| | GGCGGCGGATCGTCCCAGTTCAGAGTGTCCCCTCTGGACCGCACCT |
| | GGAACCTGGGCGAGACCGTGGAGCTGAAATGTCAGGTCCTGCTGAG |
| | CAACCCGACCTCCGGGTGCAGTTGGCTGTTCCAGCCGCGTGGTGCT |
| | GCCGCAAGCCCTACGTTCCTGCTTTACCTGAGCCAGAACAAGCCCAA |
| | GGCGGCCGAGGGCCTGGACACCCAGAGATTCTCCGGCAAGCGCCT |
| | GGGGGACACATTCGTGCTTACTTTGAGCGATTTCCGCAGAGAGAAC |
| | GAGGGCTACTATTTCTGTTCGGCGCTGAGCAATTCCATCATGTATTTC |
| | AGCCACTTTGTGCCAGTGTTCCTGCCTGCCAAGCCTACCACAACACC |
| | AGCTCCCCGTCCCCCGACTCCGGCGCCTACCATCGCGAGTCAACCG |
| | TTGAGCCTGAGGCCTGAGGCTTGTCGGCCCGCTGCGGGGGGTGCC |
| | GTCCACACCAGGGGCCTCGACTTTGCGTGCGACATCTATATTTGGGC |
| | GCCTCTGGCGGGTACCTGCGGGGTGCTGCTGCTGTCATTGGTGATT |
| | ACCCTGTACTGCAATCACCGCAACCGCCGGCGGGTCTGTAAGTGCC |
| | CACGGCCTGTGGTCAAGTCCGGTGACAAACCGTCGCTCTCGGCTCG |
| | CTACGTGCGCGCTAAGCGCAGCGGTTCCGGGGCCACCAACTTTT |
| | CATTGCTGAAGCAGGCCGGTGATGTGGAGGAGAATCCAGGGCC |
| | CATGCGCCCCAGGCTTTGGCTCCTTCTTGCTGCTCAGCTCACTGT |
| | CTTGCATGGCAACTCCGTTCTGCAGCAGACTCCCGCCTACATCA |
| | AGGTGCAGACGAACAAGATGGTGATGCTGTCATGCGAGGCCAA |
| | GATCTCTCTTTCAAATATGAGAATTTATTGGCTACGACAGCGCC |
| | AGGCCCCCTCCAGCGACAGCCACCACGAGTTCCTGGCGCTTTGG |
| | GATTCTGCTAAAGGCACCATCCATGGAGAGGAGGTGGAACAGG |
| | AGAAGATAGCTGTCTTCCGCGACGCATCCCGCTTCATCCTGAAC |
| | CTGACCAGCGTGAAGCCGGAGGACAGCGGCATCTACTTCTGTAT |
| | GATCGTTGGCTCCCCCGAGCTGACCTTCGGCAAAGGCACCCAGC |
| | TGTCCGTGGTGGACTTCCTGCCCACCACAGCCCAGCCAACCAAG |
| | AAATCCACCCTCAAGAAGCGCGTGTGCCGACTGCCCCGCCCTGA |
| | AACCCAGAAGGGCCCTCTGTGCTCCCCCATCACCCTTGGACTGC |
| | TGGTGGCGGGAGTCCTGGTGCTGCTCGTATCTCTGGGTGTCGCC |
| | ATCCACCTGTGCTGCCGCCGCCGCCGCGCCCGCCTGAGGTTTAT |
| | GAAACAGTTTTACAAGTGATAAATCGATGGAAGGGTGGCATCCC |
| | TGTGACCCCTCCCCAGTGCCTCTCCTGGCCCTGGAAGTTGCCACT |
| | CCAGTGCCCACCAGCCTTGTCCTAATAAAATTAAGTTGCATCATT |
| | TTGTCTGACTAGGTGTCCTTCTATAATATTATGGGGTGGAGGGG |
| | GGTGGTATGGAGCAAGGGGCAAGTTGGGAAGACAACCTGTAGG |
| | GCCTGCGGGGTCTATTGGGAACCAAGCTGGAGTGCAGTGGCACA |
| | ATCTTGGCTCACTGCAATCTCCGCCTCCTGGGTTCAAGCGATTCT |
| | CCTGCCTCAGCCTCCCGAGTTGTTGGGATTCCAGGCATGCATGA |
| | CCAGGCTCAGCTAATTTTTGTTTTTTTGGTAGAGACGGGGTTTCA |
| | CCATATTGGCCAGGCTGGTCTCCAACTCCTAATCTCAGGTGATCT |
| | ACCCACCTTGGCCTCCCAAATTGCTGGGATTACAGGCGTGAACC |
| | ACTGCTCCCTTCCCTGTCCTTCTGATTACTAGTGGCTCCGGTGCC |
| | CGTCAGTGGGCAGAGCGCACATCGCCCACAGTCCCCGAGAAGTT |
| | GTGGGGAGGGGTCGGCAATTGAACCGGTGCCTAGAGAAGGTGG |
| | CGCGGGGTAAACTGGGAAAGTGATGTCGTGTACTGGCTCCGCCT |
| | TTTTCCCGAGGGTGGGGGAGAACCGTATATAAGTGCAGTAGTCG |
| | CCGTGAACGTTCTTTTTCGCAACGGGTTTGCCGCCAGAACACAG |
| | GTAAGTGCCGTGTGTGGTTCCCGCGGGCCTGGCCTCTTTACGGG |
| | TTATGGCCCTTGCGTGCCTTGAATTACTTCCACCTGGCTGCAGTA |
| | CGTGATTCTTGATCCCGAGCTTCGGGTTGGAAGTGGGTGGGAGA |
| | GTTCGAGGCCTTGCGCTTAAGGAGCCCCTTCGCCTCGTGCTTGA |
| | GTTGAGGCCTGGCCTGGGCGCTGGGGCCGCCGCGTGCGAATCTG |
| | GTGGCACCTTCGCGCCTGTCTCGCTGCTTTCGATAAGTCTCTAGC |
| | CATTTAAAATTTTTGATGACCTGCTGCGACGCTTTTTTTCTGGCA |
| | AGATAGTCTTGTAAATGCGGGCCAAGATCTGCACACTGGTATTT |
| | CGGTTTTTGGGGCCGCGGGCGGCGACGGGGCCCGTGCGTCCCAG |
| | CGCACATGTTCGGCGAGGCGGGGCCTGCGAGCGCGGCCACCGA |
| | GAATCGGACGGGGGTAGTCTCAAGCTGGCCGGCCTGCTCTGGTG |
| | CCTGGCCTCGCGCCGCCGTGTATCGCCCCGCCCTGGGCGGCAAG |
| | GCTGGCCCGGTCGGCACCAGTTGCGTGAGCGGAAAGATGGCCG |
| | CTTCCCGGCCCTGCTGCAGGGAGCTCAAAATGGAGGACGCGGCG |
| | CTCGGGAGAGCGGGCGGGTGAGTCACCCACACAAAGGAAAAGG |
| | GCCTTTCCGTCCTCAGCCGTCGCTTCATGTGACTCCACGGAGTAC |
| | CGGGCGCCGTCCAGGCACCTCGATTAGTTCTCGAGCTTTTGGAG |
| | TACGTCGTCTTTAGGTTGGGGGGAGGGGTTTTATGCGATGGAGT |
| | TTCCCCACACTGAGTGGGTGGAGACTGAAGTTAGGCCAGCTTGG |
| | CACTTGATGTAATTCTCCTTGGAATTTGCCCTTTTTGAGTTTGGA |
| | TCTTGGTTCATTCTCAAGCCTCAGACAGTGGTTCAAAGTTTTTTT |
| | CTTCCATTTCAGGTGTCGTGAACTAGTCCAGTGTGGTGGAATTCT |
| | GCAGATATCACGGCTAGCGCCACCATGGGTCGGGGGCTGCTCAG |
| | GGGCCTGTGGCCGCTGCACATCGTCCTGTGGACGCGTATCGCCA |
| | GCACGATCCCACCGCACGTTCAGAAGTCGGTGAATAACGACATG |
| | ATAGTCACTGACAACAACGGTGCAGTCAAGTTTCCACAACTGTG |
| | TAAATTTTGTGATGTGAGATTTTCCACCTGTGACAACCAGAAAT |
| | CCTGCATGAGCAACTGCAGCATCACCTCCATCTGTGAGAAGCCA |
| | CAGGAAGTCTGTGTGGCTGTATGGAGAAAGAATGACGAGAACA |
| | TAACACTAGAGACAGTTTGCCATGACCCCAAGCTCCCCTACCAT |
| | GACTTTATTCTGGAAGATGCTGCTTCTCCAAAGTGCATTATGAA |
| | GGAGAAGAAAAAGCCTGGTGAGACTTTCTTCATGTGTTCCTGTA |
| | GCTCTGATGAGTGCAATGACAACATCATCTTCTCAGAAGAATAT |
| | AACACCAGCAATCCTGACTTGTTGCTAGTCATATTTCAAGTGAC |
| | AGGCATCAGCCTCCTGCCACCACTGGGAGTTGCCATATCTGTCA |
| | TCATCATCTTCTACTGCTACCGCGTGAACCGGCAGCAGAAGGCT |
| | AGTGGTTCAGGCGCAACGAATTTCTCTTTGCTGAAGCAGGCTGG |
| | GGATGTCGAAGAAAATCCGGGTCCAATGGTGGGCTCGCTCAACT |
| | GCATCGTAGCAGTCTCCCAGAATATGGGCATCGGGAAGAACGGT |
| | GATTTCCCGTGGCCCCCACTTCGCAACGAGAGCCGTTATTTCCA |
| | AAGAATGACTACAACCTCCTCCGTGGAGGGTAAGCAGAACCTG |
| | GTCATCATGGGGAAGAAGACCTGGTTCTCTATCCCTGAAAAAAA |
| | CCGCCCCCTGAAGGGCCGCATCAACCTGGTGCTGAGCAGGGAAC |
| | TCAAGGAGCCTCCTCAGGGCGCGCATTTTCTGAGCCGCTCATTG |
| | GATGACGCTCTCAAACTGACCGAACAGCCGGAGCTAGCCAACA |
| | AGGTGGACATGGTGTGGATCGTCGGAGGCTCCTCCGTGTACAAG |
| | GAGGCCATGAATCACCCCGGCCACTTGAAGCTGTTCGTCACCCG |
| | GATCATGCAGGACTTCGAGTCGGACACGTTCTTTCCAGAGATTG |
| | ACCTGGAGAAGTACAAGCTGCTGCCCGAGTACCCGGGAGTTCTT |
| | AGTGATGTGCAGGAGGAGAAAGGCATCAAGTACAAATTTGAGG |
| | TGTACGAGAAGAACGACTAACGGTCCGTCCTGACCAATGCTGGA |
| | GTTCTTCGCCCACCCCAACTTGTTTATTGCAGCTTATAATGGTTA |
| | CAAATAAAGCAATAGCATCACAAATTTCACAAATAAAGCATTTT |
| | TTTCACTGCATTCTAGTTGTGGTTTGTCCAAACTCATCAATGTAT |
| | CTTATCATGTCTGTATACAGGTTACCTCAGTCTCCTAGGTACGTC |
| | TTATATCTATGAAAAAACATTCAAAAGCACAACATCTAGAAGAA |
| | CTTACCTTTTTTCACCACTCTATTGCAAAGATATGTACCGATTTC |
| | TCTCGAAGTACAAAAAACCGCTAGTTTTCAAATTCACCTCAAGA |
| | CTTTGAAAAAAAATTGAATCTGTCAATGTCAAATAAAATCAGAA |
| | ACAAATGTCATAATGTTACGTTAATGTTGTCAGGTCGAAAAATA |
| | AAATTGCAAATAGAAATTTTGTTCCTTTTTTATTGGTTTTTATTG |
| | GTGGGAAAAATATTCCCTCTAACTGCAAAAGGGTTAATTATGTT |
| | AGAGGTAGAGTCGACAAGCTT |
| | Map of the pNVVD160_TSC-200-A02_TCR-28_MSCV-TCR28-CD8- |
| | EF1a-TGFR-DHFR Vector |
| | [symbol] |
| | Key: CD: cluster of differentiation RNA-OUT: anti-sense RNA against the |
| | bacterial levansucrase encoded by sacB. SV: simian virus TCR: T Cell |
| | Receptor, TIR: terminal inverted repeat, QBend: Mouse anti Human CD34 |
| | antibody, dnTGFbRII: Dominant-negative TGF beta Receptor II, DHFR: |
| | Dihydrofolate reductase selection marker |

*For vectors in Table 2, the MSCV promoter is in bold. Beta chain is annotated using bold and italic text. Alpha chain is annotated using bold and underlined text. CD34 enrichment tag (e.g., Q tag) is annotated using italic and underlined text. CD8-alpha is in italic. CD8-beta is underlined.
*Included in Tables 1 and 2 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Table 1 or 2, or a portion thereof. Such nucleic acid molecules can have a function of the full-length nucleic acid as described further herein.

TABLE 3
Antigenic epitopes
MAGEA1 epitopes
presented by HLA serotype HLA-C*07
Peptide Epitopes
VRFFFPSL (SEQ ID NO: 52)
FFFPSLREA (SEQ ID NO: 53)
ARVRFFFPSL (SEQ ID NO: 54)
VRFFFPSLR (SEQ ID NO: 55)
RVRFFFPSL (SEQ ID NO: 56)
FFPSLREA (SEQ ID NO: 57)
ARVRFFFPSLR (SEQ ID NO: 58)
SARVRFFF (SEQ ID NO: 59)
VRFFFPSLREA (SEQ ID NO: 60)
RFFFPSLREA (SEQ ID NO: 61)
MAGEC2 epitopes
presented by HLA serotype HLA-B*07
Peptide Epitopes
RAREFMEL (SEQ ID NO: 62)
RAREFMELL (SEQ ID NO: 63)
RAREFMELLF (SEQ ID NO: 64)
LKRAREFMEL (SEQ ID NO: 65)
VILKRAREF (SEQ ID NO: 66)
FPVILKRAR (SEQ ID NO: 67)
KRAREFMEL (SEQ ID NO: 68)
KRAREFMELL (SEQ ID NO: 69)
LKRAREFMELL (SEQ ID NO: 70)
RAREFMELLFG (SEQ ID NO: 71)
HPV16 E7 epitopes
presented by HLA serotype HLA-A*02
Peptide Epitopes
YMLDLQPET (SEQ ID NO: 72)
YMLDLQPETT (SEQ ID NO: 72a)
MAGEA1 epitopes presented
by HLA serotype HLA-A*02 (e.g., HLA-A*02:01)
Peptide Epitopes
KVLEYVIKV (SEQ ID NO: 83)
VLEYVIKV (SEQ ID NO: 83a)
KVLEYVIK (SEQ ID NO: 83b)
* Included in Table 3, such as Table 3A, Table 3B, Table 3C, and Table 3D, are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequence listed in Table 3, such as Table 3A, Table 3B, Table 3C, and Table 3D, or a portion thereof. Such polypeptides may have a function of the full-length peptide or polypeptide as described further herein.

### Example 3: Representative, Non-Limiting Combination Therapy Example

The present Example provides compositions and methods useful for multiplexed TCR-T cell therapy, including an anti-MAGE-A1 TCR and an anti-PRAME TCR. Without wishing to be bound by any particular scientific theory, the present Example further includes that multiplexed TCR-T cell therapy mimics a natural oligoclonal T cell response to cancer. Multiplexed TCR-T cell therapy (e.g., including an anti-MAGE-A1 TCR and an anti-PRAME TCR) provides for methods and compositions that address certain challenges associated with treating solid tumors.

Various assays can be used to confirm the utility of a multiplexed TCR-T cell therapy (e.g., multiplexed TCR-T cell therapy that includes an anti-MAGE-A1 TCR (such as “TCR 1479”, which is also known as “MAGE-A1-1479,” “1479”. “TSC-204-A02”, and “TSC-204-A0201”) and an anti-PRAME TCR (such as “TCR 366”, which is also known as “366” and “TSC-203-A02” (also known as “TSC-203-A0201”), and/or “TCR 358”, which is also known as “358”)). In a representative, non-limiting example, an anti-MAGE-A1 TCR, an anti-PRAME TCR, and one or more target cell lines expressing their cognate antigens are multiplexed using direct and indirect co-culture experiments to evaluate the potential synergy of using more than one TCR to target tumors as well as understanding the biological mechanisms behind such synergy. This assay can be used to model multiplexed T cell-mediated cancer killing by a multiplexed TCR-T cell therapy that includes an anti-MAGE-A1 TCR and an anti-PRAME TCR, and heterogeneity, in vitro.

In one representative case, for example, multiplexing of (i) an anti-MAGE-A1 TCR targeting an HLA-A*02 serotype-restricted epitope of MAGE-A1 (Table 5, e.g., SEQ ID NO: 83) and (ii) an anti-PRAME TCR targeting an HLA-A*02 serotype-restricted epitope of PRAME (Table 7, e.g., SEQ ID NO: 104), such as by using engineered cells expressing such TCRs, can be used and/or tested. In some embodiments, pan-T cells are transduced and selected to express the relevant TCRs (anti-MAGE-A1 or anti-PRAME). Target cells are a mixture of two cell lines, each expressing only one of the two antigens. U266B1 cells are HLA-A*02: 01+ and MAGE-A1+. Hs695T, A375, and NCI-H1563 cells are HLA-A*02: 01+ and PRAME+. Both cell lines are engineered to express Incucyte®NucLight Red and mixed together to mimic tumor heterogeneity. Engineered T cells or non-engineered donor control T-cells (Control TCR-T) are co-cultured with Incucyte® NucLight Red-labeled target cell lines at indicated effector cell to target cell (E: T) ratios, and their survival can be quantified on an IncuCyte® as a readout of cytotoxicity of the T cells.

An anti-MAGE-A1 TCR encompassed by the present invention can be a TCR that includes (e.g., comprises, consist essentially of, or consists of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 4; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 4.

An anti-MAGE-A1 TCR encompassed by the present invention can be a TCR that includes (e.g., comprises, consist essentially of, or consists of): a) a TCR alpha chain sequence selected from the group consisting of the TCR alpha chain sequences listed in Table 4) a TCR beta chain sequence selected from the group consisting of the TCR beta chain sequences listed in Table 4.

An anti-MAGE-A1 TCR encompassed by the present invention can be a TCR that includes (e.g., comprises, consist essentially of, or consists of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 4; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR VB domain sequences listed in Table 4.

An anti-MAGE-A1 TCR encompassed by the present invention can be a TCR that includes (e.g., comprises, consist essentially of, or consists of): a) a TCR alpha chain variable (V_{α}) domain sequence selected from the group consisting of the TCR V_{α} domain sequences listed in Table 4; and/or b) a TCR beta chain variable (V_{β}) domain sequence selected from the group consisting of the TCR VB domain sequences listed in Table 4.

An anti-MAGE-A1 TCR encompassed by the present invention can be a TCR that includes (e.g., comprises, consist essentially of, or consists of): at least one (e.g., one, two or three, such as CDR3 alone or in combination with a CDR1 and CDR2) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain CDR sequence selected from the group consisting of the TCR alpha chain CDR sequences listed in Table 4. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 4, each CDR3 having a sequence homology as recited in this paragraph, are provided.

An anti-MAGE-A1 TCR encompassed by the present invention can be a TCR that includes (e.g., comprises, consist essentially of, or consists of) at least one (e.g., one, two or three, such as CDR3 alone or in combination with a CDR1 and CDR2) TCR beta chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain CDR sequence selected from the group consisting of the TCR beta chain CDR sequences listed in Table 4. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 4, each CDR3 having a sequence homology as recited in this paragraph, are provided.

An anti-MAGE-A1 TCR encompassed by the present invention can be a TCR that includes (e.g., comprises, consist essentially of, or consists of) at least one (e.g., one, two or three)) TCR alpha chain complementarity determining region (CDR) listed in Table 4.

An anti-MAGE-A1 TCR encompassed by the present invention can be a TCR that includes (e.g., comprises, consist essentially of, or consists of) at least one (e.g., one, two or three)) TCR beta chain complementarity determining region (CDR) listed in Table 4.

An anti-MAGE-A1 TCR encompassed by the present invention can be a TCR that includes (e.g., comprises, consist essentially of, or consists of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{α} sequence listed in Table 4.

An anti-MAGE-A1 TCR encompassed by the present invention can be a TCR that includes (e.g., comprises, consist essentially of, or consists of) a TCR beta chain constant region (CB) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Table 4.

An anti-MAGE-A1 TCR encompassed by the present invention can be a TCR that includes (e.g., comprises, consist essentially of, or consists of) a TCR alpha chain constant region (C_{α}) sequence selected from the group consisting of the TCR Ca sequences listed in Table 4.

An anti-MAGE-A1 TCR encompassed by the present invention can be a TCR that includes (e.g., comprises, consist essentially of, or consists of) a TCR beta chain constant region (CB) sequence selected from the group consisting of the TCR CB sequences listed in Table 4.

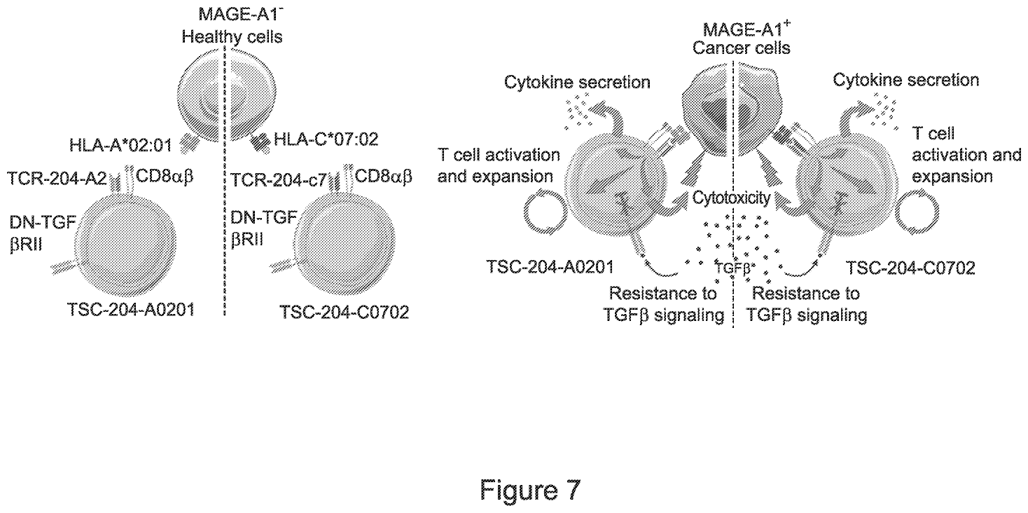

TABLE 4
TCR sequences recognizing a MAGEA1 antigen presented by HLA serotype
HLA-A*02

| | |
|---|---|
| MAGEA1- | Alpha chain DNA sequence |
| 278-1479 WT | ATGGAAAAAATGCTCGAGTGCGCCTTCATCGTGCTTTGGCTGCA |
| sequence | GCTCGGATGGCTGAGCGGAGAGGATCAAGTGACACAGTCTCCC |
| Alpha chain: | GAGGCTCTGAGGCTGCAAGAGGGCGAAAGCAGCTCCCTGAATT |
| TRAV20/TRA | GCAGCTACACCGTGTCTGGCCTGAGGGGCCTGTTTTGGTACAG |
| J26/TRAC | ACAAGACCCTGGCAAGGGACCCGAGTTCCTGTTCACACTGTAC |
| | TCTGCCGGCGAAGAAAAAGAGAAAGAGCGCCTGAAAGCAACCCT |
| | GACCAAGAAAGAGAGCTTCCTGCACATCACAGCCCCTAAGCCAG |
| | AGGACAGCGCTACTTACCTGTGTGCCGTTTCATACGGCCAGAAT |
| | TTCGTTTTTGGTCCCGGAACCAGATTGTCCGTGCTGCCCTatat |
| | ccagaaccctgaccctgccgtgtaccagctgagagactctaaatccagtgacaagtctgtctgcctattcacc |
| | gattttgattctcaaacaaatgtgtcacaaagtaaggattctgatgtgtatatcacagacaaaactgtgctag |
| | acatgaggtctatggacttcaagagcaacagtgctgtggcctggagcaacaaatctgactttgcatgtgcaaa |
| | cgccttcaacaacagcattattccagaagacaccttcttccccagcccagaaagttcctgtgatgtcaagctg |
| | gtcgagaaaagctttgaaacagatacgaacctaaactttcaaaacctgtcagtgattgggttccgaatcctcc |
| | tcctgaaagtggccgggtttaatctgctcatgacgctgcggctgtggtccagc |
| | (SEQ ID NO: 73) |
| | Alpha chain protein sequence |
| | MEKMLECAFIVLWLQLGWLSGEDQVTQSPEALRLQEGESSSLNCS |
| | YTVSGLRGLFWYRQDPGKGPEFLFTLYSAGEEKEKERLKATLTKK |
| | ESFLHITAPKPEDSATYLCAVSYGQNFVFGPGTRLSVLPYiqnpdpavy |
| | qlrdskssdksvelftdfdsqtnvsqskdsdvyitdktvldmrsmdfksnsavawsnksdfacanafn |
| | nsiipedtffpspesscdvklveksfetdtninfqnlsvigfrilllkvagfnllmtlrlwss |
| | (SEQ ID NO: 74) |
| MAGEA1- | Beta chain DNA sequence |
| 278-1479 WT | ATGGGACCCAGGCTCCTCTTCTGGGCACTGCTTTGTCTCCTCGGA |
| sequence | ACAGGCCCAGTGGAGGCTGGAGTCACACAAAGTCCCACACACC |
| Beta chain: | TGATCAAAACGAGAGGACAGCAAGCGACTCTGAGATGCTCTCCT |
| TRBV5- | ATCTCTGGGCACACCAGTGTGTACTGGTACCAACAGGCCCTGG |
| 8/TRBJ1- | GTCTGGGCCTCCAGTTCCTCCTTTGGTATGACGAGGGTGAAGA |
| 1/TRBC1 | GAGAAACAGAGGAAACTTCCCTCCTAGATTTTCAGGTCGCCAGT |
| | TCCCTAATTATAGCTCTGAGCTGAATGTGAACGCCTTGGAGCTG |
| | GAGGACTCGGCCCTGTATCTCTGTGCTTCCTCACTTGGGCAAT |
| | TGAACACAGAGGCATTCTTTGGACAAGGCACCAGACTCACAGT |
| | TGTAGaggacctgaacaaggtgttcccacccgaggtcgctgtgtttgagccatcagaagcagagatct |
| | cccacacccaaaaggccacactggtgtgcctggccacaggcttcttccctgaccacgtggagctgagc |
| | tggtgggtgaatgggaaggaggtgcacagtggggtcagcacggacccgcagcccctcaaggagcagcc |
| | cgccctcaatgactccagatactgcctgagcagccgcctgagggtctcggccaccttctggcagaacc |
| | cccgcaaccacttcgctgtcaagtccagttctacgggctctcggagaatgacgagtggacccaggata |
| | gggccaaacccgtcacccagatcgtcagcgccgaggcctggggtagagcagactgtggctttacctcg |
| | gtgtcctaccagcaaggggtcctgtctgccaccatcctctatgagatcctgctagggaaggccaccct |
| | gtatgctgtgctggtcagcgcccttgtgttgatggccatggtcaagagaaaggatttc |
| | (SEQ ID NO: 75) |
| | Beta chain protein sequence |
| | MGPRLLFWALLCLLGTGPVEAGVTQSPTHLIKTRGQQATLRCSPI |
| | SGHTSVYWYQQALGLGLQFLLWYDEGEERNRGNFPPRESGRQFPN |
| | YSSELNVNALELEDSALYLCASSLGQLNTEAFFGQGTRLTVVEdi |
| | nkvfppevavfepseaeishtqkatlvclatgffpdhvelswwvngkevhsgvstdpqplkeqpalnd |
| | sryclssrlrvsatfwqnprnhfreqvqfyglsendewtqdrakpvtqivsaeawgradegftsvsyq |
| | qgvlsatilyeillgkatlyavlvsalvlmamvkrkdf |
| | (SEQ ID NO: 76) |
| MAGEA1- | Alpha chain DNA sequence |
| 278-1479 | ATGGAAAAAATGCTCGAGTGCGCCTTCATCGTGCTTTGGCTGCA |
| MGTM codon | GCTCGGATGGCTGAGCGGAGAGGATCAAGTGACACAGTCTCCC |
| optimized | GAGGCTCTGAGGCTGCAAGAGGGCGAAAGCAGCTCCCTGAATT |
| sequence (also | GCAGCTACACCGTGTCTGGCCTGAGGGGCCTGTTTTGGTACAG |
| known as | ACAAGACCCTGGCAAGGGACCCGAGTTCCTGTTCACACTGTACT |
| clone | CTGCCGGCGAAGAAAAAGAGAAAGAGCGCCTGAAAGCAACCC |
| “MAGE-A1- | TGACCAAGAAAGAGAGCTTCCTGCACATCACAGCCCCTAAGCCA |
| 1479”, “TCR | GAGGACAGCGCTACTTACCTGTGTGCCGTTTCATACGGCCAGA |
| 1479”, | ATTTCGTTTTTGGTCCCGGAACCAGATTGTCCGTGCTGCCCTacat |
| “1479”, | ccagaaccccgaccccgccgtgtaccagctgagggactccaagtccagcgacaagagcgtgtgtctgtt |
| “TSC-204- | tacggacttcgacagccagaccaacgtgagtcaaagcaaggacagcgacgtctacataacggataagac |
| A02”, and | cgtgctggacatgcggagcatggacttcaagagcaacagcgccgtggcctggtccaacaagagcgactt |
| “TSC-204- | cgcctgcgccaacgccttcaacaacagcatcatccccgaggacaccttcttccccagcagcgacgtgcc |
| A0201”) | ctgcgacgtgaaactggtggagaagtccttcgagacagacaccaatctgaactttcagaacctgctggt |
| Note: This | gatcgtgctgcggattctgctgctgaaagtggccggcttcaatctgctgatgaccctgcggctgtggag |
| clone was used | c (SEQ ID NO: 77) |
| in FIG. 7-9 | Alpha chain protein sequence |
| as the | MEKMLECAFIVLWLQLGWLSGEDQVTQSPEALRLQEGESSSLNCS |
| MAGEA1- | YTVSGLRGLFWYRQDPGKGPEFLFTLYSAGEEKEKERLKATLTKK |
| A02 TCR in | ESFLHITAPKPEDSATYLCAVSYGQNFVFGPGTRLSVLPYiqnpdpavy |
| multiplex with | qlrdskssdksvclftdfdsqtnvsqskdsdvyitdktvldmrsmdfksnsavawsnksdfacanafn |
| the MAGEA1- | nsiipedtffpssdvpcdvklveksfetdtnlnfqnllvivlrilllkvagfnllmtlrlws |
| C07 TCR | (SEQ ID NO: 78) |
| Alpha chain: | Beta chain DNA sequence |
| TRAV20/TRA | ATGGGACCCAGGCTCCTCTTCTGGGCACTGCTTTGTCTCCTCGGA |
| J26/MGTM | ACAGGCCCAGTGGAGGCTGGAGTCACACAAAGTCCCACACACC |
| modified | TGATCAAAACGAGAGGACAGCAAGCGACTCTGAGATGCTCTCCT |
| TRAC | ATCTCTGGGCACACCAGTGTGTACTGGTACCAACAGGCCCTGG |
| MAGEA1- | GTCTGGGCCTCCAGTTCCTCCTTTGGTATGACGAGGGTGAAGA |
| 278-1479 | GAGAAACAGAGGAAACTTCCCTCCTAGATTTTCAGGTCGCCAGT |
| MGTM codon | TCCCTAATTATAGCTCTGAGCTGAATGTGAACGCCTTGGAGCTG |
| optimized | GAGGACTCGGCCCTGTATCTCTGTGCTTCCTCACTTGGGCAAT |
| sequence (also | TGAACACAGAGGCATTCTTTGGACAAGGCACCAGACTCACAGT |
| known as | TGTAGaagatctgaacaaggtgttccctccagaggtggccgtgttcgagccttctaaggccgagatcgcc |
| clone | cacacacaaaaagccaccctcgtgtgcctggccaccggctttttccccgaccacgtggaactgtcttggt |
| “MAGE-A1- | gggtcaacggcaaagaggtgcactccggcgtgtcaacggatccccagcctctgaaagaacagcctgccc |
| 1479”, “TCR | tgaacgacagccggtactgcctgagctccagactgagagtgtccgccaccttctggcagaacccccggaa |
| 1479”, | ccacttcagatgccaggtgcagttttacggcctgagcgagaacgacgagtggacccaggacagagccaa |
| “1479” | gcccgtgacacaaatcgtgtctgccgaagcctggggaagagccgattgcggcatcaccagcgcctcctat |
| “TSC-204- | caccagggcgtgctgagcgccacaatcctgtacgaaatcctgctgggcaaggccaccctgtacgccgtg |
| A02”, and | ctggtgtctgctctggtgctgatggccatggtcaagcggaaggactttggcagcggcagagccaaaaggt |
| “TSC-204- | ccgggagcggt (SEQ ID NO: 79) |
| A0201”) | Beta chain protein sequence |
| Note: This | MGPRLLFWALLCLLGTGPVEAGVTQSPTHLIKTRGQQATLRCSPI |
| clone was used | SGHTSVYWYQQALGLGLQFLLWYDEGEERNRGNFPPRFSGRQFPN |
| in FIG. 7-9 | YSSELNVNALELEDSALYLCASSLGQLNTEAFFGQGTRLTVVEdl |
| as the | nkvfppevavfepskaeiahtqkatlvclatgffpdhvelswwvngkevhsgvstdpqplkeqpalndsr |
| MAGEA1- | yclssrlrvsatfwqnprnhfrcqvqfyglsendewtqdrakpvtqivsaeawgradcgitsasyhqgv |
| A02 TCR in | lsatilyeillgkatlyavlvsalvlmamvkrkdfgsgrakrsgsg (SEQ ID NO: 80) |
| multiplex with | |
| the MAGEA1- | |
| C07 TCR | |
| Beta chain: | |
| TRBV5- | |
| 8/TRBJ1- | |
| 1/MGTM | |
| modified | |
| TRBC | |
| Complete Beta | ATGGGACCCAGGCTCCTCTTCTGGGCACTGCTTTGTCTCCTCGGA |
| and Alpha | ACAGGCCCAGTGGAGGCTGGAGTCACACAAAGTCCCACACACC |
| ORF DNA | TGATCAAAACGAGAGGACAGCAAGCGACTCTGAGATGCTCTCCT |
| Sequence (The | ATCTCTGGGCACACCAGTGTGTACTGGTACCAACAGGCCCTGG |
| underlined | GTCTGGGCCTCCAGTTCCTCCTTTGGTATGACGAGGGTGAAGA |
| italic region in | GAGAAACAGAGGAAACTTCCCTCCTAGATTTTCAGGTCGCCAGT |
| the “Furin- | TCCCTAATTATAGCTCTGAGCTGAATGTGAACGCCTTGGAGCTG |
| P2A” site | GAGGACTCGGCCCTGTATCTCTGTGCTTCCTCACTTGGGCAAT |
| encodes a | TGAACACAGAGGCATTCTTTGGACAAGGCACCAGACTCACAGT |
| sequence | TGTAGaagatctgaacaaggtgttccctccagaggtggccgtgttcgagccttctaaggccgagatcgcc |
| allowing for | cacacacaaaaagccaccctcgtgtgcctggccaccggctttttccccgaccacgtggaactgtcttggt |
| expression of | gggtcaacggcaaagaggtgcactccggcgtgtcaacggatccccagcctctgaaagaacagcctgccct |
| two | gaacgacagccggtactgcctgagctccagactgagagtgtccgccaccttctggcagaacccccggaac |
| polypeptide | cacttcagatgccaggtgcagttttacggcctgagcgagaacgacgagtggacccaggacagagccaagc |
| chains in a | ccgtgacacaaatcgtgtctgccgaagcctggggaagagccgattgcggcatcaccagcgcctcctatca |
| single cassette) | ccagggcgtgctgagcgccacaatcctgtacgaaatcctgctgggcaaggccaccctgtacgccgtgCt |
| | ggtgtctgctctggtgctgatggccatggtcaagcggaaggactttggcagcggcagagccaaaag |
| | tccgggagcggtGCGACAAACTTTAGCCTGTTGAAACAAGCCGGCGACGT |
| | TGAAGAGAACCCCGGACCTATGGAAAAAATGCTCGAGTGCGCCTT |
| | CATCGTGCTTTGGCTGCAGCTCGGATGGCTGAGCGGAGAGGATC |
| | AAGTGACACAGTCTCCCGAGGCTCTGAGGCTGCAAGAGGGCGA |
| | AAGCAGCTCCCTGAATTGCAGCTACACCGTGTCTGGCCTGAGG |
| | GGCCTGTTTTGGTACAGACAAGACCCTGGCAAGGGACCCGAGTT |
| | CCTGTTCACACTGTACTCTGCCGGCGAAGAAAAAGAGAAAGA |
| | GCGCCTGAAAGCAACCCTGACCAAGAAAGAGAGCTTCCTGCAC |
| | ATCACAGCCCCTAAGCCAGAGGACAGCGCTACTTACCTGTGTGC |
| | CGTTTCATACGGCCAGAATTTCGTTTTTGGTCCCGGAACCAGA |
| | TTGTCCGTGCTGCCCTacatccagaaccccgaccccgccgtgtaccagctgagggactcca |
| | agtccagcgacaagagcgtgtgtctgtttacggacttcgacagccagaccaacgtgagtcaaagcaagga |
| | cagcgacgtctacataacggataagaccgtgctggacatgcggagcatggacttcaagagcaacagcgc |
| | cgtggcctggtccaacaagagcgacttcgcctgcgccaacgccttcaacaacagcatcatccccgaggac |
| | accttcttccccagcagcgacgtgccctgcgacgtgaaactggtggagaagtccttcgagacagacacca |
| | atctgaactttcagaacctgctggtgatcgtgctgcggattctgctgctgaaagtggccggcttcaatctgct |
| | gatgaccctgcggctgtggagc (SEQ ID NO: 81) |
| Complete Beta | MGPRLLFWALLCLLGTGPVEAGVTQSPTHLIKTRGQQATLRCSPIS |
| and Alpha | GHTSVYWYQQALGLGLQFLLWYDEGEERNRGNFPPRFSGRQFPN |
| ORF Protein | YSSELNVNALELEDSALYLCASSLGQLNTEAFFGQGTRLTVVEdInk |
| Sequence (The | vfppevavfepskaeiahtqkatlvclatgffpdhvelswwvngkevhsgvstdpqplkeqpalndsr |
| underlined | yclssrlrvsatfwqnprnhfrcqvqfyglsendewtqdrakpvtqivsaeawgradcgitsasyhqgv |
| italic region in | LsatilyeillgkatlyavlvsalvlmamvkrkdfgsgrakrsgsgATNFSLLKQAGDVEENP |
| the “Furin- | GPMEKMLECAFIVLWLQLGWLSGEDQVTQSPEALRLQEGESSSLN |
| P2A” site | CSYTVSGLRGLFWYRQDPGKGPEFLFTLYSAGEEKEKERLKATLT |
| allows | KKESFLHITAPKPEDSATYLCAVSYGQNFVFGPGTRLSVLPYiqnpdp |
| expression of | avyqlrdskssdksvclftdfdsqtnvsqskdsdvyitdktvldmrsmdfksnsavawsnksdfacan |
| two | afnnsiipedtffpssdvpcdvklveksfetdtnlnfqnllvivlrilllkvagfnllmtlrlws |
| polypeptide | (SEQ ID NO: 82) |
| chains in a | |
| single cassette) | |

* Table 4 provides, in part, representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well- known IMGT nomenclature described herein. Similarly, CDR1 and CDR2 of TRAV and TRBV are well-known in the art since they are based on well-known and annotated TRAV and TRBV sequences (e.g., as annotated in databases like IMGT available at imt.org and IEDB available at iedb.org).
* For certain depicted vectors, MSCV promoter is in bold. Beta chain is annotated using bold and italic text. Alpha chain is annotated using bold and underlined text. CD34-enrichment tag (Q tag) is annotated using italic and underlined text. CD8-alpha is in italic. CD8-beta is underlined.
* Table 4 includes polypeptide sequences, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequences listed therein, or a portion thereof. Such polypeptides may have a function of the full-length peptide or polypeptide as described further herein.
* Table 4 includes RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed therein, or a portion thereof. Such nucleic acid molecules can have a function of the full-length nucleic acid as described further herein.

TABLE 5
MAGEA1 epitopes presented by HLA
serotype HLA-A*02
(e.g., HLA-A*02:01)
Peptide Epitopes

KVLEYVIKV (SEQ ID NO: 83)
VLEYVIKV (SEQ ID NO: 83a)
KVLEYVIK (SEQ ID NO: 83b)

As described above, any combination of TCRs described herein is contemplated for use.

For example, an anti-PRAME TCR encompassed by the present invention can be a TCR that includes (e.g., comprises, consist essentially of, or consists of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 6; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 6.

An anti-PRAME TCR encompassed by the present invention can be a TCR that includes (e.g., comprises, consist essentially of, or consists of): a) a TCR alpha chain sequence selected from the group consisting of the TCR alpha chain sequences listed in Table 6) a TCR beta chain sequence selected from the group consisting of the TCR beta chain sequences listed in Table 6.

An anti-PRAME TCR encompassed by the present invention can be a TCR that includes (e.g., comprises, consist essentially of, or consists of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 6; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR VB domain sequences listed in Table 6.

An anti-PRAME TCR encompassed by the present invention can be a TCR that includes (e.g., comprises, consist essentially of, or consists of): a) a TCR alpha chain variable (V_{α}) domain sequence selected from the group consisting of the TCR V_{α} domain sequences listed in Table 6; and/or b) a TCR beta chain variable (V_{β}) domain sequence selected from the group consisting of the TCR VB domain sequences listed in Table 6.

An anti-PRAME TCR encompassed by the present invention can be a TCR that includes (e.g., comprises, consist essentially of, or consists of): at least one (e.g., one, two or three, such as CDR3 alone or in combination with a CDR1 and CDR2) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain CDR sequence selected from the group consisting of the TCR alpha chain CDR sequences listed in Table 6. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 6, each CDR3 having a sequence homology as recited in this paragraph, are provided.

An anti-PRAME TCR encompassed by the present invention can be a TCR that includes (e.g., comprises, consist essentially of, or consists of) at least one (e.g., one, two or three, such as CDR3 alone or in combination with a CDR1 and CDR2) TCR beta chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain CDR sequence selected from the group consisting of the TCR beta chain CDR sequences listed in Table 6. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 6, each CDR3 having a sequence homology as recited in this paragraph, are provided.

An anti-PRAME TCR encompassed by the present invention can be a TCR that includes (e.g., comprises, consist essentially of, or consists of) at least one (e.g., one, two or three)) TCR alpha chain complementarity determining region (CDR) listed in Table 6.

An anti-PRAME TCR encompassed by the present invention can be a TCR that includes (e.g., comprises, consist essentially of, or consists of) at least one (e.g., one, two or three)) TCR beta chain complementarity determining region (CDR) listed in Table 6.

An anti-PRAME TCR encompassed by the present invention can be a TCR that includes (e.g., comprises, consist essentially of, or consists of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{α} sequence listed in Table 6.

An anti-PRAME TCR encompassed by the present invention can be a TCR that includes (e.g., comprises, consist essentially of, or consists of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Table 6.

An anti-PRAME TCR encompassed by the present invention can be a TCR that includes (e.g., comprises, consist essentially of, or consists of) a TCR alpha chain constant region (C_{α}) sequence selected from the group consisting of the TCR Ca sequences listed in Table 6.

An anti-PRAME TCR encompassed by the present invention can be a TCR that includes (e.g., comprises, consist essentially of, or consists of) a TCR beta chain constant region (C_{β}) sequence selected from the group consisting of the TCR C_{β} sequences listed in Table 6.

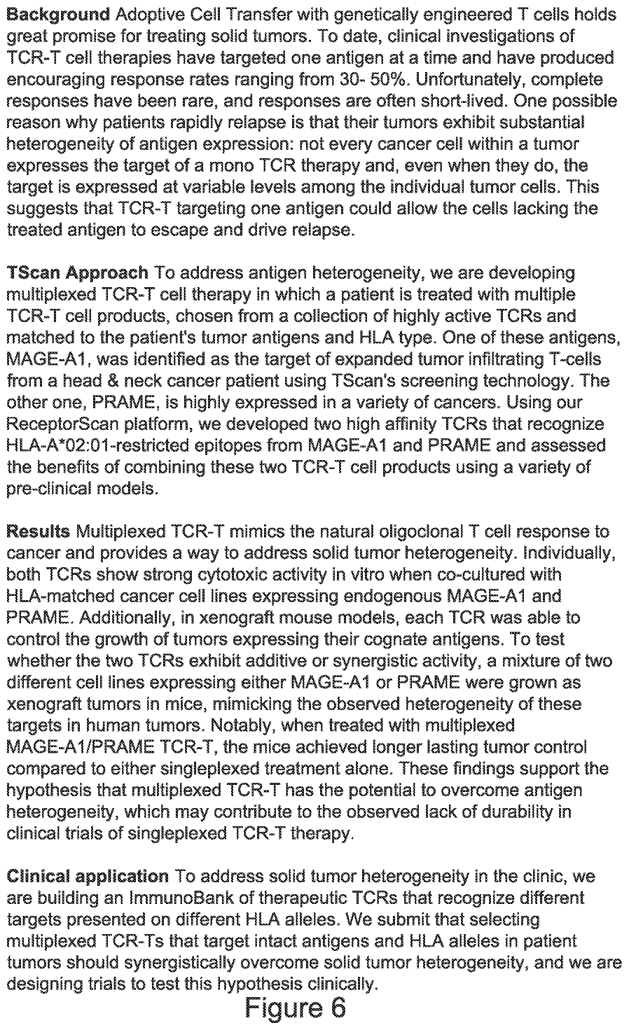

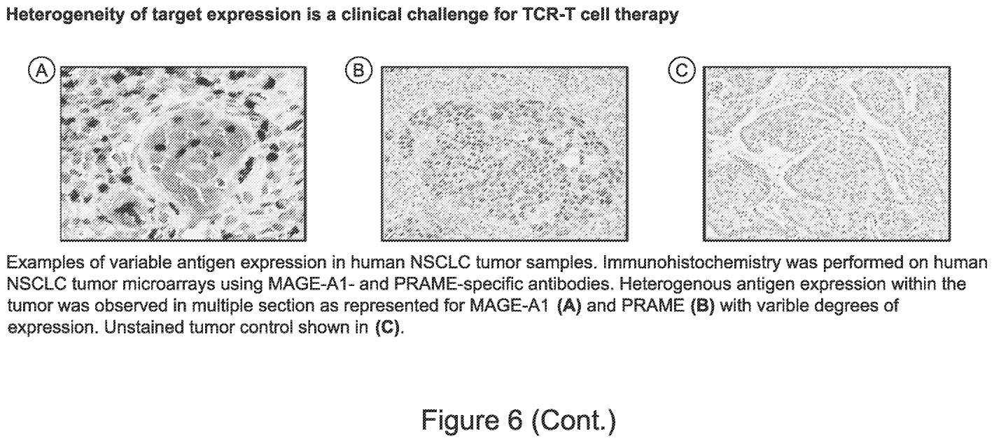

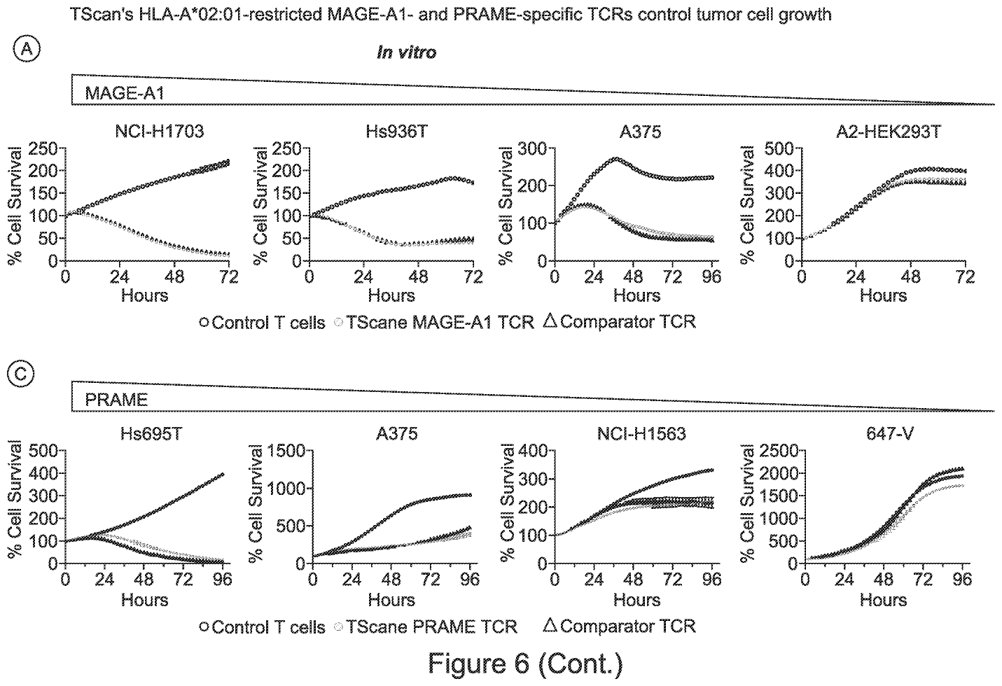

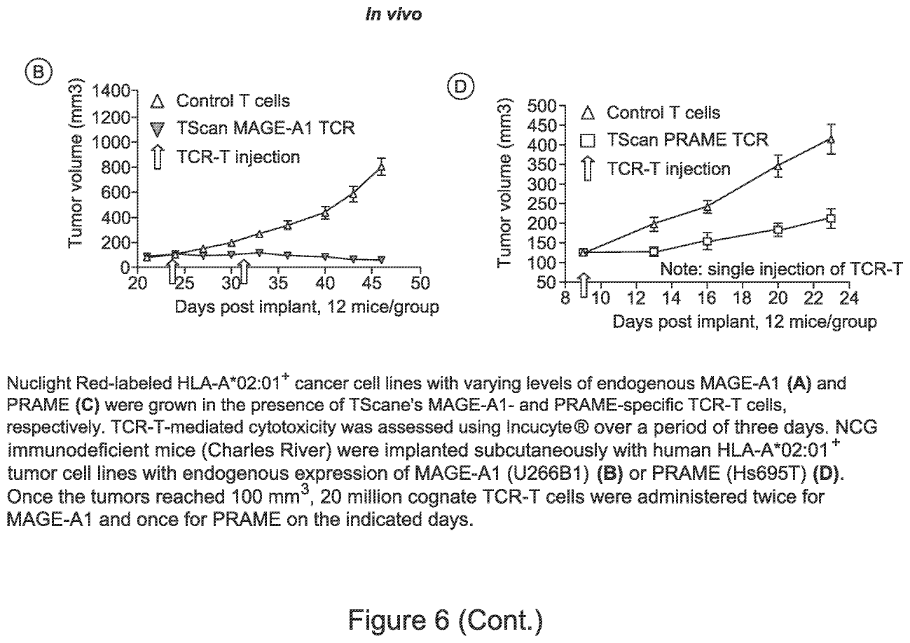

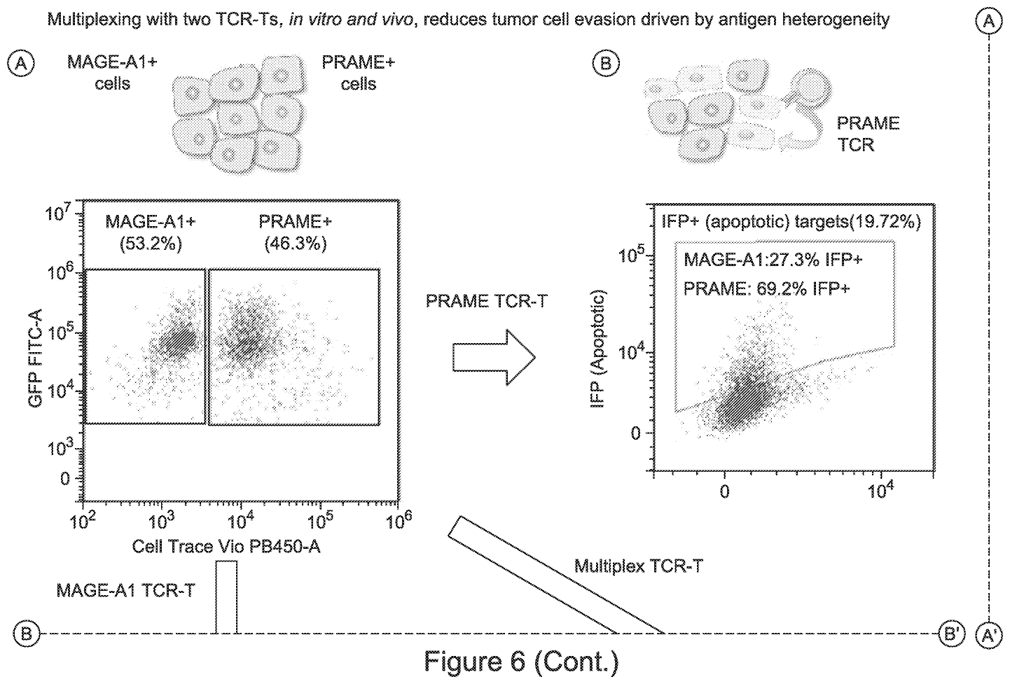

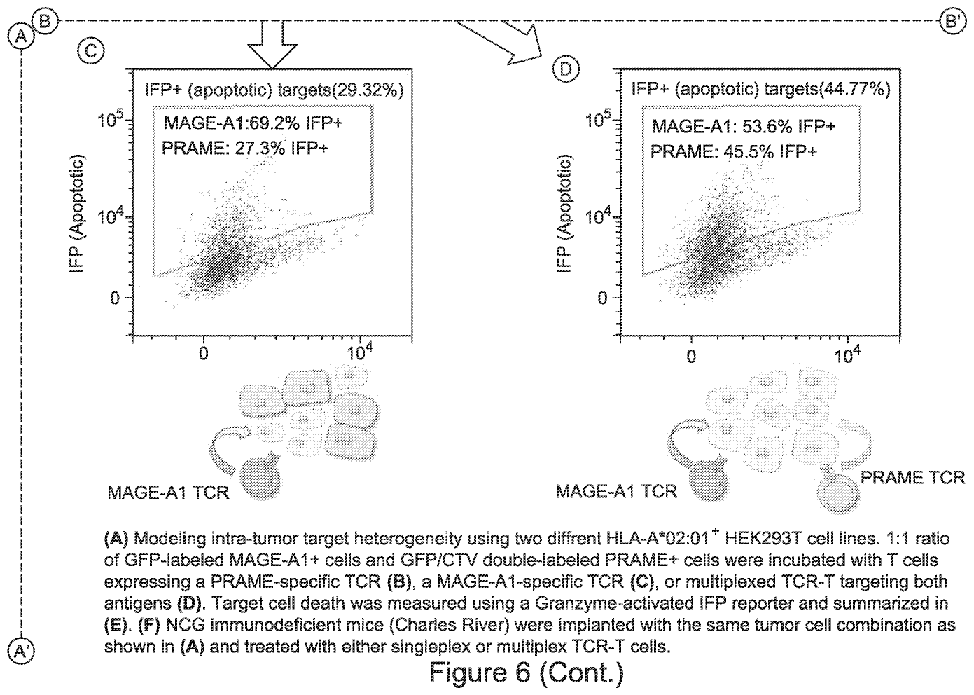

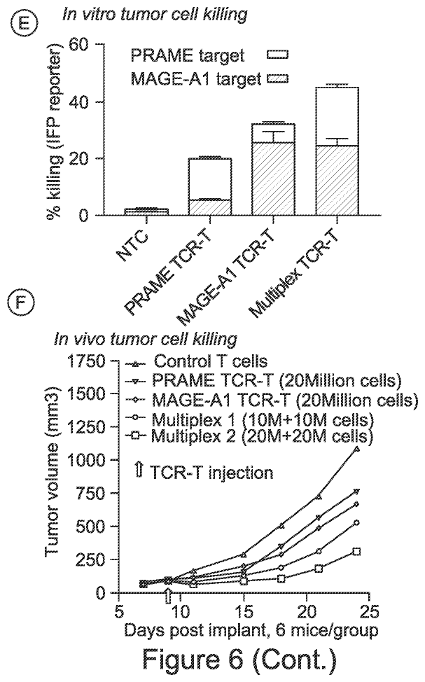

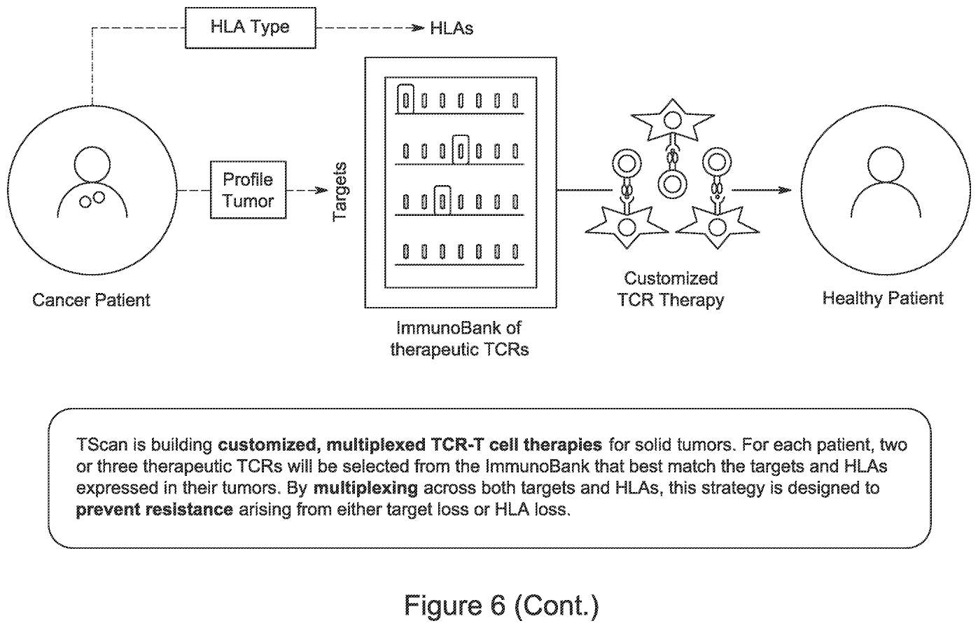

TABLE 6
TCR sequences recognizing a PRAME antigen presented by HLA serotype
HLA-A*02

| | |
|---|---|
| PRAME-425- | Alpha chain DNA sequence |
| 366 WT | ATGGCCTGTCCTGGCTTCCTGTGGGCCCTTGTGATCAGCACTTGC |
| sequence | CTGGAATTCAGCATGGCTCAGACAGTCACCCAGTCTCAGCCCGA |
| Alpha chain: | AATGAGCGTCCAAGAGGCTGAAACCGTGACTCTGTCTTGTACCT |
| TRAV38- | ACGACACCTCCGAGAGCGATTACTACCTCTTTTGGTATAAGCA |
| 2DV8/TRAJ50 | ACCGCCGTCCAGGCAAATGATCCTCGTGATCCGGCAAGAAGCT |
| /TRAC | TACAAACAGCAGAATGCTACCGAAAACCGGTTCTCCGTCAATT |
| | TTCAGAAAGCCGCTAAGAGCTTTAGCCTGAAAATCTCCGACTCT |
| | CAGCTCGGCGACGCTGCTATGTATTTCTGTGCCTACCGCAAAA |
| | CTTCTTACGATAAAGTCATTTTTGGGCCAGGGACAAGCTTATC |
| | AGTCATTCCAAatatccagaaccctgaccctgccgtgtaccagctgagagactctaaatccagtg |
| | acaagtctgtctgcctattcaccgattttgattctcaaacaaatgtgtcacaaagtaaggattctgatgtgtatat |
| | cacagacaaaactgtgctagacatgaggtctatggacttcaagagcaacagtgctgtggcctggagcaac |
| | aaatctgactttgcatgtgcaaacgccttcaacaacagcattattccagaagacaccttcttccccagcccag |
| | aaagttcctgtgatgtcaagctggtcgagaaaagctttgaaacagatacgaacctaaactttcaaaacctgtc |
| | agtgattgggttccgaatcctcctcctgaaagtggccgggtttaatctgctcatgacgctgcggctgtggtcc |
| | agc (SEQ ID NO: 84) |
| | Alpha chain protein sequence |
| | MACPGFLWALVISTCLEFSMAQTVTQSQPEMSVQEAETVTLSCTYD |
| | TSESDYYLFWYKQPPSRQMILVIRQEAYKQQNATENRFSVNFQKA |
| | AKSFSLKISDSQLGDAAMYFCAYRKTSYDKVIFGPGTSLSVIPNiqnp |
| | dpavyqlrdskssdksvclftdfdsqtnvsqskdsdvyitdktvldmrsmdfksnsavawsnksdfac |
| | anafnnsiipedtffpspesscdvklveksfetdtnlnfqnlsvigfrilllkvagfnllmtlrlwss (SEQ |
| | ID NO: 85) |
| PRAME-425- | Beta chain DNA sequence |
| 366 WT | ATGCTGAGCCCCGACCTGCCTGACAGCGCTTGGAATACCAGACT |
| sequence | CCTGTGCAGAGTGATGCTGTGCCTGCTTGGAGCTGGAAGTGTGG |
| Beta chain: | CTGCTGGTGTCATTCAGTCCCCAAGGCACCTGATCAAAGAGAAG |
| TRBV13/TRB | AGAGAGACAGCCACTCTGAAGTGCTACCCCATTCCTAGACACG |
| J2-1/TRBC1 | ACACGGTCTATTGGTATCAGCAAGGACCTGGACAGGACCCTCA |
| | GTTCCTGATCAGCTTCTACGAGAAGATGCAGAGCGACAAGGGC |
| | AGCATCCCCGACAGATTTTCTGCCCAGCAGTTCAGCGACTACCA |
| | CAGCGAGCTGAACATGAGCAGCCTGGAACTGGGCGATAGCGCC |
| | CTGTACTTCTGTGCCTCTTCTTTCGCACGCCTGGAAGGTCGC |
| | GATAATGAACAATTTTTTGGGCCAGGGACACGGCTCACCGTGC |
| | TAGaggacctgaacaaggtgttcccacccgaggtcgctgtgtttgagccatcagaagcagagatctccc |
| | acacccaaaaggccacactggtgtgcctggccacaggcttcttccctgaccacgtggagctgagctggtg |
| | ggtgaatgggaaggaggtgcacagtggggtcagcacggacccgcagcccctcaaggagcagcccgcc |
| | ctcaatgactccagatactgcctgagcagccgcctgagggtctcggccaccttctggcagaacccccgca |
| | accacttccgctgtcaagtccagttctacgggctctcggagaatgacgagtggacccaggatagggccaa |
| | acccgtcacccagatcgtcagcgccgaggcctggggtagagcagactgtggctttacctcggtgtcctac |
| | cagcaaggggtcctgtctgccaccatcctctatgagatcctgctagggaaggccaccctgtatgctgtgctg |
| | gtcagcgcccttgtgttgatggccatggtcaagagaaaggatttc (SEQ ID NO: 86) |
| | Beta chain protein sequence |
| | MLSPDLPDSAWNTRLLCRVMLCLLGAGSVAAGVIQSPRHLIKEKRE |
| | TATLKCYPIPRHDTVYWYQQGPGQDPQFLISFYEKMQSDKGSIPD |
| | RFSAQQFSDYHSELNMSSLELGDSALYFCASSFARLEGRDNEQFF |
| | GPGTRLTVLEdinkvfppevavfepseaeishtqkatlvclatgffpdhvelswwvngkevhs |
| | gvstdpqplkeqpalndsryclssrlrvsatfwqnprnhfrcqvqfyglsendewtqdrakpvtqivsa |
| | eawgradcgftsvsyqqgvlsatilyeillgkatlyavlvsalvlmamvkrkdf (SEQ ID NO: |
| | 87) |
| PRAME-425- | Alpha chain DNA sequence |
| 366 MGTM | ATGGCCTGTCCTGGCTTCCTGTGGGCCCTTGTGATCAGCACTTGC |
| codon | CTGGAATTCAGCATGGCTCAGACAGTCACCCAGTCTCAGCCCGA |
| optimized | AATGAGCGTCCAAGAGGCTGAAACCGTGACTCTGTCTTGTACCT |
| sequence (also | ACGACACCTCCGAGAGCGATTACTACCTCTTTTGGTATAAGCA |
| known as | ACCGCCGTCCAGGCAAATGATCCTCGTGATCCGGCAAGAAGCT |
| clone “TCR | TACAAACAGCAGAATGCTACCGAAAACCGGTTCTCCGTCAATT |
| 366”, “366”, | TTCAGAAAGCCGCTAAGAGCTTTAGCCTGAAAATCTCCGACTCT |
| “TSC-203- | CAGCTCGGCGACGCTGCTATGTATTTCTGTGCCTACCGCAAAA |
| A02”, and | CTTCTTACGATAAAGTCATTTTTGGGCCAGGGACAAGCTTATC |
| “TSC-203- | AGTCATTCCAAacatccagaaccccgaccccgccgtgtaccagctgagggactccaagtccag |
| A0201”) | cgacaagagcgtgtgtctgtttacggacttcgacagccagaccaacgtgagtcaaagcaaggacagcga |
| Note: This | cgtctacataacggataagaccgtgctggacatgcggagcatggacttcaagagcaacagcgccgtggc |
| clone was used | ctggtccaacaagagcgacttcgcctgcgccaacgccttcaacaacagcatcatccccgaggacaccttct |
| in FIG. 6 as | tccccagcagcgacgtgccctgcgacgtgaaactggtggagaagtccttcgagacagacaccaatctgaa |
| PRAME TCR | ctttcagaacctgctggtgatcgtgctgcggattctgctgctgaaagtggccggcttcaatctgctgatgacc |
| in multiplex | ctgcggctgtggagc (SEQ ID NO: 88) |
| with the | Alpha chain protein sequence |
| MAGEA1 | MACPGFLWALVISTCLEFSMAQTVTQSQPEMSVQEAETVTLSCTYD |
| TCR. T cells | TSESDYYLFWYKQPPSRQMILVIRQEAYKQQNATENRFSVNFQKA |
| were | AKSFSLKISDSQLGDAAMYFCAYRKTSYDKVIFGPGTSLSVIPNiqnp |
| lentivirally | dpavyqlrdskssdksvclftdfdsqtnvsqskdsdvyitdktvldmrsmdfksnsavawsnksdfac |
| transduced | anafnnsiipedtffpssdvpcdvklveksfetdtnlnfqnllvivlrilllkvagfnllmtlrlws (SEQ |
| with TCR | ID NO: 89) |
| expressed from | |
| a vector | |
| comprising | |
| MSCV- | |
| TCRalpha- | |
| TCRbeta- | |
| CD8alpha- | |
| CD8Beta | |
| (MGTM | |
| constant region | |
| in TCR and | |
| CD34 tag | |
| fused to CD8). | |
| Alpha chain: | |
| TRAV38- | |
| 2DV8/TRAJ50 | |
| /MGTM | |
| modified | |
| TRAC | |
| PRAME-425- | Beta chain DNA sequence |
| 366 MGTM | ATGCTGAGCCCCGACCTGCCTGACAGCGCTTGGAATACCAGACT |
| codon | CCTGTGCAGAGTGATGCTGTGCCTGCTTGGAGCTGGAAGTGTGG |
| optimized | CTGCTGGTGTCATTCAGTCCCCAAGGCACCTGATCAAAGAGAAG |
| sequence (also | AGAGAGACAGCCACTCTGAAGTGCTACCCCATTCCTAGACACG |
| known as | ACACGGTCTATTGGTATCAGCAAGGACCTGGACAGGACCCTCA |
| clone “TCR | GTTCCTGATCAGCTTCTACGAGAAGATGCAGAGCGACAAGGGC |
| 366”, “366”, | AGCATCCCCGACAGATTTTCTGCCCAGCAGTTCAGCGACTACCA |
| “TSC-203- | CAGCGAGCTGAACATGAGCAGCCTGGAACTGGGCGATAGCGCC |
| A02”, and | CTGTACTTCTGTGCCTCTTCTTTCGCACGCCTGGAAGGTCGC |
| “TSC-203- | GATAATGAACAATTTTTTGGGCCAGGGACACGGCTCACCGTGC |
| A0201”) | TAGaagatctgaacaaggtgttccctccagaggtggccgtgttcgagccttctaaggccgagatcgccc |
| Note: This | acacacaaaaagccaccctcgtgtgcctggccaccggctttttccccgaccacgtggaactgtcttggtgg |
| clone was used | gtcaacggcaaagaggtgcactccggcgtgtcaacggatccccagcctctgaaagaacagcctgccctg |
| in FIG. 6 as | aacgacagccggtactgcctgagctccagactgagagtgtccgccaccttctggcagaacccccggaac |
| PRAME TCR | cacttcagatgccaggtgcagtittacggcctgagcgagaacgacgagtggacccaggacagagccaag |
| in multiplex | cccgtgacacaaatcgtgtctgccgaagcctggggaagagccgattgcggcatcaccagcgcctcctatc |
| with the | accagggcgtgctgagcgccacaatcctgtacgaaatcctgctgggcaaggccaccctgtacgccgtgct |
| MAGEA1 | ggtgtctgctctggtgctgatggccatggtcaagcggaaggactttggcagcggcagagccaaaaggtcc |
| TCR. T cells | gggagcggt (SEQ ID NO: 90) |
| were | Beta chain protein sequence |
| lentivirally | MLSPDLPDSAWNTRLLCRVMLCLLGAGSVAAGVIQSPRHLIKEKRE |
| transduced | TATLKCYPIPRHDTVYWYQQGPGQDPQFLISFYEKMQSDKGSIPD |
| with TCR | RFSAQQFSDYHSELNMSSLELGDSALYFCASSFARLEGRDNEQFF |
| expressed from | GPGTRLTVLEdlnkvfppevavfepskaeiahtqkatlvclatgffpdhvelswwvngkevhs |
| a vector | gvstdpqplkeqpalndsryclssrlrvsatfwqnprnhfrcqvqfyglsendewtqdrakpvtqivsa |
| comprising | eawgradcgitsasyhqgvlsatilyeillgkatlyavlvsalvlmamvkrkdfgsgrakrsgsg |
| MSCV- | (SEQ ID NO: 91) |
| TCRalpha- | |
| TCRbeta- | |
| CD8alpha- | |
| CD8Beta | |
| (MGTM | |
| constant region | |
| in TCR and | |
| CD34 tag | |
| fused to CD8). | |
| Beta chain: | |
| TRBV13/TRB | |
| J2-1/MGTM | |
| modified | |
| TRBC | |
| Complete Beta | ATGCTGAGCCCCGACCTGCCTGACAGCGCTTGGAATACCAGACT |
| and Alpha | CCTGTGCAGAGTGATGCTGTGCCTGCTTGGAGCTGGAAGTGTGG |
| ORF DNA | CTGCTGGTGTCATTCAGTCCCCAAGGCACCTGATCAAAGAGAAG |
| Sequence (The | AGAGAGACAGCCACTCTGAAGTGCTACCCCATTCCTAGACACG |
| underlined | ACACGGTCTATTGGTATCAGCAAGGACCTGGACAGGACCCTCA |
| italic region in | GTTCCTGATCAGCTTCTACGAGAAGATGCAGAGCGACAAGGGC |
| the “Furin- | AGCATCCCCGACAGATTTTCTGCCCAGCAGTTCAGCGACTACCA |
| P2A” site | CAGCGAGCTGAACATGAGCAGCCTGGAACTGGGCGATAGCGCC |
| encodes a | CTGTACTTCTGTGCCTCTTCTTTCGCACGCCTGGAAGGTCGC |
| sequence | GATAATGAACAATTTTTTGGGCCAGGGACACGGCTCACCGTGC |
| allowing for | TAGaagatctgaacaaggtgttccctccagaggtggccgtgttcgagccttctaaggccgagatcgccc |
| expression of | acacacaaaaagccaccctcgtgtgcctggccaccggctttttccccgaccacgtggaactgtcttggtgg |
| two | gtcaacggcaaagaggtgcactccggcgtgtcaacggatccccagcctctgaaagaacagcctgccctg |
| polypeptide | aacgacagccggtactgcctgagctccagactgagagtgtccgccaccttctggcagaacccccggaac |
| chains in a | cacttcagatgccaggtgcagttttacggcctgagcgagaacgacgagtggacccaggacagagccaag |
| single cassette) | cccgtgacacaaatcgtgtctgccgaagcctggggaagagccgattgcggcatcaccagcgcctcctatc |
| | accagggcgtgctgagcgccacaatcctgtacgaaatcctgctgggcaaggccaccctgtacgccgtgct |
| | ggtgtctgctctggtgctgatggccatggtcaagcggaaggactttggcagcggcagagccaaaaggtc |
| | cgggagcggtGCGACAAACTTTAGCCTGTTGAAACAAGCCGGCGACGTT |
| | GAAGAGAACCCCGGACCTATGGCCTGTCCTGGCTTCCTGTGGGCC |
| | CTTGTGATCAGCACTTGCCTGGAATTCAGCATGGCTCAGACAGT |
| | CACCCAGTCTCAGCCCGAAATGAGCGTCCAAGAGGCTGAAACC |
| | GTGACTCTGTCTTGTACCTACGACACCTCCGAGAGCGATTACT |
| | ACCTCTTTTGGTATAAGCAACCGCCGTCCAGGCAAATGATCCTC |
| | GTGATCCGGCAAGAAGCTTACAAACAGCAGAATGCTACCGAA |
| | AACCGGTTCTCCGTCAATTTTCAGAAAGCCGCTAAGAGCTTTAG |
| | CCTGAAAATCTCCGACTCTCAGCTCGGCGACGCTGCTATGTATTT |
| | CTGTGCCTACCGCAAAACTTCTTACGATAAAGTCATTTTTGG |
| | GCCAGGGACAAGCTTATCAGTCATTCCAAacatccagaaccccgaccccgcc |
| | gtgtaccagctgagggactccaagtccagcgacaagagcgtgtgtctgtttacggacttcgacagccaga |
| | ccaacgtgagtcaaagcaaggacagcgacgtctacataacggataagaccgtgctggacatgcggagca |
| | tggacttcaagagcaacagcgccgtggcctggtccaacaagagcgacttcgcctgcgccaacgccttcaa |
| | caacagcatcatccccgaggacaccttcttccccagcagcgacgtgccctgcgacgtgaaactggtggag |
| | aagtccttcgagacagacaccaatctgaactttcagaacctgctggtgatcgtgctgcggattctgctgctga |
| | aagtggccggcttcaatctgctgatgaccctgcggctgtggagc (SEQ ID NO: 92) |
| Complete Beta | MLSPDLPDSAWNTRLLCRVMLCLLGAGSVAAGVIQSPRHLIKEKRE |
| and Alpha | TATLKCYPIPRHDTVYWYQQGPGQDPQFLISFYEKMQSDKGSIPD |
| ORF Protein | RFSAQQFSDYHSELNMSSLELGDSALYFCASSFARLEGRDNEQFF |
| Sequence (The | GPGTRLTVLEdlnkvfppevavfepskaeiahtqkatlvclatgffpdhvelswwvngkevhs |
| underlined | gvstdpqplkeqpalndsryclssrlrvsatfwqnprnhfrcqvqfyglsendewtqdrakpvtqivsa |
| italic region in | eawgradcgitsasyhqgvlsatilyeillgkatlyavlvsalvlmamvkrkdfgsgrakrsgsgATN |
| the “Furin- | FSLLKQAGDVEENPGPMACPGFLWALVISTCLEFSMAQTVTQSQPE |
| P2A“” site | MSVQEAETVTLSCTYDTSESDYYLFWYKQPPSRQMILVIRQEAYK |
| allows | QQNATENRFSVNFQKAAKSFSLKISDSQLGDAAMYFCAYRKTSYD |
| expression of | KVIFGPGTSLSVIPNiqnpdpavyqlrdskssdksvclftdfdsqtnvsqskdsdvyitdktvl |
| two | dmrsmdfksnsavawsnksdfacanafnnsiipedtffpssdvpcdvklveksfetdtnlnfqnllviv |
| polypeptide | Irilllkvagfnllmtlrlws (SEQ ID NO: 93) |
| chains in a | |
| single cassette) | |
| PRAME-425- | Alpha chain DNA sequence |
| 358 WT | ATGGAAACCCTGCTGAAGGTGCTGTCTGGCACCCTGCTGTGGCA |
| sequence | GCTGACATGGGTCCGATCTCAGCAGCCTGTGCAGTCTCCTCAGG |
| Alpha chain: | CCGTGATTCTGAGAGAAGGCGAGGACGCCGTGATCAACTGCAG |
| TRAV30/TRA | CAGCTCTAAGGCCCTGTACAGCGTGCACTGGTACAGGCAGAAA |
| J20/TRAC | CACGGCGAGGCCCCAGTGTTTCTGATGATTCTGCTGAAAGGCG |
| | GCGAGCAGAAGGGCCACGATAAGATCTCCGCCAGCTTCAACGA |
| | GAAGAAGCAGCAGTCCAGCCTGTACCTGACAGCCAGCCAGCTG |
| | AGCTACAGCGGCACCTATTTCTGTGGCACAGAAGGTACTGGTG |
| | ACTACAAGCTCTCTTTTGGAGCCGGAACCACAGTAACTGTAAG |
| | AGCAAatatccagaaccctgaccctgccgtgtaccagctgagagactctaaatccagtgacaagtctg |
| | tctgcctattcaccgattttgattctcaaacaaatgtgtcacaaagtaaggattctgatgtgtatatcacagaca |
| | aaactgtgctagacatgaggtctatggacttcaagagcaacagtgctgtggcctggagcaacaaatctgac |
| | tttgcatgtgcaaacgccttcaacaacagcattattccagaagacaccttcttccccagcccagaaagttcct |
| | gtgatgtcaagctggtcgagaaaagctttgaaacagatacgaacctaaactttcaaaacctgtcagtgattg |
| | ggttccgaatcctcctcctgaaagtggccgggtttaatctgctcatgacgctgcggctgtggtccagc |
| | (SEQ ID NO: 94) |
| | Alpha chain protein sequence |
| | METLLKVLSGTLLWQLTWVRSQQPVQSPQAVILREGEDAVINCSSS |
| | KALYSVHWYRQKHGEAPVFLMILLKGGEQKGHDKISASFNEKKQ |
| | QSSLYLTASQLSYSGTYFCGTEGTGDYKLSFGAGTTVTVRANiqnp |
| | dpavyqlrdskssdksvclftdfdsqtnvsqskdsdvyitdktvldmrsmdfksnsavawsnksdfac |
| | anafnnsiipedtffpspesscdvklveksfetdtninfqnlsvigfrilllkvagfnlimtlrlwss (SEQ |
| | ID NO: 95) |
| PRAME-425- | Beta chain DNA sequence |
| 358 WT | ATGGGACCTCAGCTGCTGGGATATGTGGTGCTGTGTCTGCTCGG |
| sequence | AGCTGGACCCCTGGAAGCTCAAGTGACACAGAACCCCAGATAC |
| Beta chain: | CTGATCACCGTGACCGGCAAAAAGCTGACCGTGACCTGTAGCCA |
| TRBV27/TRB | GAACATGAACCACGAGTACATGAGCTGGTATCGGCAAGACCCT |
| J2-7/TRBC1 | GGCCTGGGGCTGAGACAGATCTACTATAGCATGAACGTGGAAG |
| | TGACCGACAAAGGCGACGTGCCCGAGGGCTATAAGGTGTCCCG |
| | GAAAGAGAAGCGGAACTTTCCACTGATCCTGGAATCCCCATCTC |
| | CTAACCAGACCAGCCTGTATTTTTGCGCTAGTTCTGCCGGGAC |
| | CGGGGGGCATGAGCAATACTTCGGGCCGGGCACCAGGCTCAC |
| | GGTCACAGaggacctgaacaaggtgttcccacccgaggtcgctgtgtttgagccatcagaagcagag |
| | atctcccacacccaaaaggccacactggtgtgcctggccacaggcttcttccctgaccacgtggagctg |
| | agctggtgggtgaatgggaaggaggtgcacagtggggtcagcacggacccgcagcccctcaaggagc |
| | agcccgccctcaatgactccagatactgcctgagcagccgcctgagggtctcggccaccttctggcagaa |
| | cccccgcaaccacttccgctgtcaagtccagttctacgggctctcggagaatgacgagtggacccaggat |
| | agggccaaacccgtcacccagatcgtcagcgccgaggcctggggtagagcagactgtggctttacctcg |
| | gtgtcctaccagcaaggggtcctgtctgccaccatcctctatgagatcctgctagggaaggccaccctgtat |
| | gctgtgctggtcagcgcccttgtgttgatggccatggtcaagagaaaggatttc (SEQ ID NO: 96) |
| | Beta chain protein sequence |
| | MGPQLLGYVVLCLLGAGPLEAQVTQNPRYLITVTGKKLTVTCSQN |
| | MNHEYMSWYRQDPGLGLRQIYYSMNVEVTDKGDVPEGYKVSRK |
| | EKRNFPLILESPSPNQTSLYFCASSAGTGGHEQYFGPGTRLTVTEdln |
| | kvfppevavfepseaeishtqkatlvclatgffpdhvelswwvngkevhsgvstdpqplkeqpalnds |
| | ryclssrlrvsatfwqnprnhfreqvqfyglsendewtqdrakpvtqivsaeawgradcgftsvsyqqg |
| | vlsatilyeillgkatlyavlvsalvlmamvkrkdf (SEQ ID NO: 97) |
| PRAME-425- | Alpha chain DNA sequence |
| 358 MGTM | ATGGAAACCCTGCTGAAGGTGCTGTCTGGCACCCTGCTGTGGCA |
| codon | GCTGACATGGGTCCGATCTCAGCAGCCTGTGCAGTCTCCTCAGG |
| optimized | CCGTGATTCTGAGAGAAGGCGAGGACGCCGTGATCAACTGCAG |
| sequence (also | CAGCTCTAAGGCCCTGTACAGCGTGCACTGGTACAGGCAGAAA |
| known as | CACGGCGAGGCCCCAGTGTTTCTGATGATTCTGCTGAAAGGCG |
| clone “TCR | GCGAGCAGAAGGGCCACGATAAGATCTCCGCCAGCTTCAACGA |
| 358”) | GAAGAAGCAGCAGTCCAGCCTGTACCTGACAGCCAGCCAGCTG |
| Alpha chain: | AGCTACAGCGGCACCTATTTCTGTGGCACAGAAGGTACTGGTG |
| TRAV30/TRA | ACTACAAGCTCTCTTTTGGAGCCGGAACCACAGTAACTGTAAG |
| J20/MGTM | AGCAAacatccagaaccccgaccccgccgtgtaccagctgagggactccaagtccagcgacaaga |
| modified | gcgtgtgtctgtttacggacttcgacagccagaccaacgtgagtcaaagcaaggacagcgacgtctacata |
| TRAC | acggataagaccgtgctggacatgcggagcatggacttcaagagcaacagcgccgtggcctggtccaac |
| | aagagcgacttcgcctgcgccaacgccttcaacaacagcatcatccccgaggacaccttcttccccagca |
| | gcgacgtgccctgcgacgtgaaactggtggagaagtccttcgagacagacaccaatctgaactttcagaa |
| | cctgctggtgatcgtgctgcggattctgctgctgaaagtggccggcttcaatctgctgatgaccctgcggct |
| | gtggagc (SEQ ID NO: 98) |
| | Alpha chain protein sequence |
| | METLLKVLSGTLLWQLTWVRSQQPVQSPQAVILREGEDAVINCSSS |
| | KALYSVHWYRQKHGEAPVFLMILLKGGEQKGHDKISASFNEKKQ |
| | QSSLYLTASQLSYSGTYFCGTEGTGDYKLSFGAGTTVTVRANiqnp |
| | dpavyqlrdskssdksvclftdfdsqtnvsqskdsdvyitdktvldmrsmdfksnsavawsnksdfac |
| | anafnnsiipedtffpssdvpcdvklveksfetdtnlnfqnllvivlrilllkvagfnllmtlrlws (SEQ |
| | ID NO: 99) |
| PRAME-425- | Beta chain DNA sequence |
| 358 MGTM | ATGGGACCTCAGCTGCTGGGATATGTGGTGCTGTGTCTGCTCGG |
| codon | AGCTGGACCCCTGGAAGCTCAAGTGACACAGAACCCCAGATAC |
| optimized | CTGATCACCGTGACCGGCAAAAAGCTGACCGTGACCTGTAGCCA |
| sequence (also | GAACATGAACCACGAGTACATGAGCTGGTATCGGCAAGACCCT |
| known as | GGCCTGGGGCTGAGACAGATCTACTATAGCATGAACGTGGAAG |
| clone “TCR | TGACCGACAAAGGCGACGTGCCCGAGGGCTATAAGGTGTCCCG |
| 358”) | GAAAGAGAAGCGGAACTTTCCACTGATCCTGGAATCCCCATCTC |
| Beta chain: | CTAACCAGACCAGCCTGTATTTTTGCGCTAGTTCTGCCGGGAC |
| TRBV27/TRB | CGGGGGGCATGAGCAATACTTCGGGCCGGGCACCAGGCTCAC |
| J2-7/MGTM | GGTCACAGaagatctgaacaaggtgttccctccagaggtggccgtgttcgagccttctaaggccga |
| modified | gatcgcccacacacaaaaagccaccctcgtgtgcctggccaccggctttttccccgaccacgtggaactgt |
| TRBC | cttggtgggtcaacggcaaagaggtgcactccggcgtgtcaacggatccccagcctctgaaagaacagc |
| | ctgccctgaacgacagccggtactgcctgagctccagactgagagtgtccgccaccttctggcagaaccc |
| | ccggaaccacttcagatgccaggtgcagitttacggcctgagcgagaacgacgagtggacccaggacag |
| | agccaagcccgtgacacaaatcgtgtctgccgaagcctggggaagagccgattgoggcatcaccaggc |
| | ctcctatcaccagggcgtgctgagcgccacaatcctgtacgaaatcctgctgggcaaggccaccctgtac |
| | gccgtgctggtgtctgctctggtgctgatggccatggtcaagcggaaggactttggcagcggcagagcca |
| | aaaggtccgggagcggt (SEQ ID NO: 100) |
| | Beta chain protein sequence |
| | MGPQLLGYVVLCLLGAGPLEAQVTQNPRYLITVTGKKLTVTCSQN |
| | MNHEYMSWYRQDPGLGLRQIYYSMNVEVTDKGDVPEGYKVSRK |
| | EKRNFPLILESPSPNQTSLYFCASSAGTGGHEQYFGPGTRLTVTEdln |
| | kvfppevavfepskaeiahtqkatlvclatgffpdhvelswwvngkevhsgvstdpqplkeqpalnds |
| | ryclssrlrvsatfwqnprnhfrcqvqfyglsendewtqdrakpvtqivsaeawgradcgitsasyhqg |
| | vlsatilyeillgkatlyavlvsalvlmamvkrkdfgsgrakrsgsg (SEQ ID NO: 101) |
| Complete Beta | ATGGGACCTCAGCTGCTGGGATATGTGGTGCTGTGTCTGCTCGG |
| and Alpha | AGCTGGACCCCTGGAAGCTCAAGTGACACAGAACCCCAGATAC |
| ORF DNA | CTGATCACCGTGACCGGCAAAAAGCTGACCGTGACCTGTAGCCA |
| Sequence (The | GAACATGAACCACGAGTACATGAGCTGGTATCGGCAAGACCCT |
| underlined | GGCCTGGGGCTGAGACAGATCTACTATAGCATGAACGTGGAAG |
| italic region in | TGACCGACAAAGGCGACGTGCCCGAGGGCTATAAGGTGTCCCG |
| the “Furin- | GAAAGAGAAGCGGAACTTTCCACTGATCCTGGAATCCCCATCTC |
| P2A” site | CTAACCAGACCAGCCTGTATTTTTGCGCTAGTTCTGCCGGGAC |
| encodes a | CGGGGGGCATGAGCAATACTTCGGGCCGGGCACCAGGCTCAC |
| sequence | GGTCACAGaagatctgaacaaggtgttccctccagaggtggccgtgttcgagccttctaaggccga |
| allowing for | gatcgcccacacacaaaaagccaccctcgtgtgcctggccaccggctttttccccgaccacgtggaactgt |
| expression of | cttggtgggtcaacggcaaagaggtgcactccggcgtgtcaacggatccccagcctctgaaagaacagc |
| two | ctgccctgaacgacagccggtactgcctgagctccagactgagagtgtccgccaccttctggcagaaccc |
| polypeptide | ccggaaccacttcagatgccaggtgcagttttacggcctgagcgagaacgacgagtggacccaggacag |
| chains in a | agccaagcccgtgacacaaatcgtgtctgccgaagcctggggaagagccgattgcggcatcaccagcgc |
| single cassette) | ctcctatcaccagggcgtgctgagcgccacaatcctgtacgaaatcctgctgggcaaggccaccctgtac |
| | gccgtgctggtgtctgctctggtgctgatggccatggtcaagcggaaggactttggcagcggcagagcca |
| | aaaggtccgggagcggtGCGACAAACTTTAGCCTGTTGAAACAAGCCGGCG |
| | ACGTTGAAGAGAACCCCGGACCTATGGAAACCCTGCTGAAGGTGC |
| | TGTCTGGCACCCTGCTGTGGCAGCTGACATGGGTCCGATCTCAG |
| | CAGCCTGTGCAGTCTCCTCAGGCCGTGATTCTGAGAGAAGGCGA |
| | GGACGCCGTGATCAACTGCAGCAGCTCTAAGGCCCTGTACAGC |
| | GTGCACTGGTACAGGCAGAAACACGGCGAGGCCCCAGTGTTTCT |
| | GATGATTCTGCTGAAAGGCGGCGAGCAGAAGGGCCACGATAA |
| | GATCTCCGCCAGCTTCAACGAGAAGAAGCAGCAGTCCAGCCTGT |
| | ACCTGACAGCCAGCCAGCTGAGCTACAGCGGCACCTATTTCTGT |
| | GGCACAGAAGGTACTGGTGACTACAAGCTCTCTTTTGGAGCC |
| | GGAACCACAGTAACTGTAAGAGCAAacatccagaaccccgaccccgccgtgtac |
| | cagctgagggactccaagtccagcgacaagagcgtgtgtctgtttacggacttcgacagccagaccaacg |
| | tgagtcaaagcaaggacagcgacgtctacataacggataagaccgtgctggacatgcggagcatggactt |
| | caagagcaacagcgccgtggcctggtccaacaagagcgacttcgcctgcgccaacgccttcaacaacag |
| | catcatccccgaggacaccttcttccccagcagcgacgtgccctgcgacgtgaaactggtggagaagtcc |
| | ttcgagacagacaccaatctgaactttcagaacctgctggtgatcgtgctgcggattctgctgctgaaagtg |
| | gccggcttcaatctgctgatgaccctgcggctgtggagc (SEQ ID NO: 102) |
| Complete Beta | MGPQLLGYVVLCLLGAGPLEAQVTQNPRYLITVTGKKLTVTCSQN |
| and Alpha | MNHEYMSWYRQDPGLGLRQIYYSMNVEVTDKGDVPEGYKVSRK |
| ORF Protein | EKRNFPLILESPSPNQTSLYFCASSAGTGGHEQYFGPGTRLTVTEdln |
| Sequence (The | kvfppevavfepskaeiahtqkatlvclatgffpdhvelswwvngkevhsgvstdpqplkeqpalnds |
| underlined | ryclssrlrvsatfwqnprnhfrcqvqfyglsendewtqdrakpvtqivsaeawgradcgitsasyhqg |
| italic region in | vlsatilyeillgkatlyavlvsalvlmamvkrkdfgsgrakrsgsgATNFSLLKQAGDVEEN |
| the “Furin- | PGPMETLLKVLSGTLLWQLTWVRSQQPVQSPQAVILREGEDAVIN |
| P2A” site | CSSSKALYSVHWYRQKHGEAPVFLMILLKGGEQKGHDKISASFNE |
| allows | KKQQSSLYLTASQLSYSGTYFCGTEGTGDYKLSFGAGTTVTVRAN |
| expression of | iqnpdpavyqlrdskssdksvclftdfdsqtnvsqskdsdvyitdktvldmrsmdfksnsavawsnks |
| two | dfacanafnnsiipedtffpssdvpcdvklveksfetdtnlnfqnllvivlrilllkvagfnllmtlrlws |
| polypeptide | (SEQ ID NO: 103) |
| chains in a | |
| single cassette) | |

* Table 6 provides, in part, representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well- known IMGT nomenclature described herein. Similarly, CDR1 and CDR2 of TRAV and TRBV are well-known in the art since they are based on well-known and annotated TRAV and TRBV sequences (e.g., as annotated in databases like IMGT available at imt.org and IEDB available at iedb.org).
* For certain depicted vectors, MSCV promoter is in bold. Beta chain is annotated using bold and italic text. Alpha chain is annotated using bold and underlined text. CD34-enrichment tag (Q tag) is annotated using italic and underlined text. CD8-alpha is in italic. CD8-beta is underlined.
* Table 6 includes polypeptide sequences, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequences listed therein, or a portion thereof. Such polypeptides may have a function of the full-length peptide or polypeptide as described further herein.
* Table 6 includes RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed therein, or a portion thereof. Such nucleic acid molecules can have a function of the full-length nucleic acid as described further herein.

TABLE 7
PRAME epitopes presented by HLA
serotype HLA-A*02 (e.g., HLA-A*02:01)
Peptide Epitopes

SLLQHLIGL (SEQ ID NO: 104)

### Example 4: Multiplexed TCR-T Cell Therapy Targeting MAGEA1 and PRAME Enhances the Activity of Adoptive T Cell Therapy in Pre-Clinical Models

The present Example is based in part on the recognition that adoptive cell transfer with genetically engineered T cells holds great promise for treating solid tumors. Certain prior clinical investigations of TCR-engineered T cell therapies (TCR-T) have targeted one antigen at a time and, in various instances, have produced response rates ranging from 30-50%. It has been observed that complete responses to such therapies have been rare, and responses have often been short-lived. Without wishing to be bound by any particular scientific theory, one possible reason why patients rapidly relapse after responding to such therapies is that their tumors exhibit substantial heterogeneity of antigen expression: not every cancer cell within a tumor expresses the target of a mono TCR therapy and, even when they do, the target is expressed at variable levels among the individual tumor cells. This suggests that TCR-T targeting one antigen could allow the cells lacking the treated antigen to escape and drive relapse.

The present Example presents development of multiplexed TCR-T cell therapy in which a patient is treated with multiple TCR-T cell products, chosen from a collection of pre-vetted TCRs matched to the patient's tumor antigens and HLA type, as a solution to address antigen heterogeneity. As proof-of-concept, two different cancer/testis antigens targeted by two different TCRs were selected. One of these antigens, MAGEA1, was identified as the target of expanded tumor infiltrating T-cells from a head & neck cancer patient using TScan's screening technology as described in Luomo et al. (2022) Cell. S0092-8674 (22) 00723-1. The other of the antigens, PRAME, is highly expressed in a variety of cancers. The present Example includes the development of two high affinity TCRs that recognize HLA-A*02:01-restricted epitopes from MAGEA1 and PRAME (see Tables 5 and 7, respectively). Benefits of combining these two TCR-T cell products, having sequences according to Table 4 and Table 6, respectively, are assessed using a variety of pre-clinical models. For example, TSC-203-A0201 and TSC-204-A0201 TCR-T cell products, such as those expressing MG™ TCRs and are codon-optimized, may be used.

Individually, both TCRs (i.e., the TCRs that recognize MAGEA1 and PRAME, respectively) are believed to show strong cytotoxic activity in vitro when co-cultured with HLA-matched cancer cell lines expressing endogenous MAGEA1 and PRAME. Additionally, in xenograft mouse models, each TCR is believed to be able to control the growth of tumors expressing their cognate antigens and HLA.

A mixture of two different cell lines expressing either MAGEA1 or PRAME along with HLA-A*02:01 was tested in vitro or grown as xenograft tumors in mice and treated with either TCR-T tested individually, or with a mixture of the two TCR-Ts. Notably, the MAGE-specific TCR-Ts and the PRAME-specific TCR-Ts were designed to selectively target their respective target cell subset and the multiplexed MAGEA1/PRAME TCR-T were designed to simultaneously target both cancer cell subsets. When treated with multiplexed MAGEA1/PRAME TCR-T, the mice were designed to achieve longer lasting tumor control compared to TCR-T targeting a single antigen.

The findings reported in this Example and further summarized in FIG. 6 are expected to demonstrate that multiplexed TCR-T is a potent means of targeting cancer with heterogeneous target antigen expression and, hence, an advantageous approach to therapy. Without wishing to be bound by any particular scientific theory, the present Example demonstrates that multiplexed TCR-T mimics the natural oligoclonal T-cell response to cancer and has the potential to overcome antigen heterogeneity, which may contribute to the observed lack of durability in monotherapy TCR-T clinical trials. The described co-culture assays are relatively short-term in duration and it is believed that further synergistic activity (such as from cytokine-dependent phenomena described herein) may be observed in other assays, such as, for example, reducing the effector to target ratio of one or more TCRs of a combination of TCRs to show the supportive effects of the other TCR(s) on the reduced TCR(s), in longer duration studies, and the like.

FIG. 6 shows representative examples of variable antigen expression in human non-small cell lung (NSCLC) tumor samples. Immunohistochemistry was performed on human NSCLC tumor microarrays using MAGE-A1-specific antibody (clone SPM282; Abcam Cat #ab25834) or PRAME-specific antibody (clone EPR20330; Abcam Cat #ab219650). Heterogenous antigen expression within the tumor was observed in multiple sections as represented for MAGE-A1 and PRAME with variable degrees of expression. Unstained tumor control is also shown.

FIG. 6 further shows the characterization of a TCR that recognizes a MAGE-A1-derived epitope presented on HLA-A*02:01, and of a TCR that recognizes a PRAME-derived epitope presented on HLA-A*02:01. Co-cultures of TCR-T cells expressing the MAGE-A1-specific TCR with a panel of HLA-A*02:01-positive cancer cell lines presenting a range of MAGE-A1 (i.e., MCI0H1703, HJS936T, and A375) or of the PRAME-specific TCR with cells presenting a range of PRAME expression (i.e., HS695T, A375, and NCI-H15632) was conducted. Cells lines positive for HLA-A*02:01, but negative for MAGE-A1 (A2-HEK293T) or PRAME (647-V), were tested as negative controls.

The TCR-T cells used in these experiments were pan T cells engineered by lentivirus transduction. The vector contains MGTM modifications and the co-receptor CD8α and CD8β were co-delivered along with the recombinant TCR primarily to ensure recognition of the TCRs on the CD4+ fraction of the pan T cells.

The data demonstrate that each TCR-T display high potency and selectivity for cells presenting the cognate peptide/MHC (pMHC), killing the relevant target cells, while sparing the cell line negative for the targeted protein.

In vivo efficacy studies were also conducted to further test the potency of the individual TCR-T cells. Female NOD-Prkdc^{em26Cd52}Il2r^{gem26Cd22}/NjuCrl (NCG) mice were implanted subcutaneously with U266B1 cancer cells (HLA-A*02:01-positive cells expressing MAGE-A1). Animals with confirmed growing tumors (with average tumor volumes of about 100 mm³; 21 days post inoculation) were randomized into different experimental groups and received two intravenous injections of MAGE-A1 TCR-T cells (20E6 each; injected the day following randomization, and again one week later), or of donor-matched, non-engineered control T cells (20E6 each; injected the day following randomization, and again one week later). The tumor volumes were measured twice weekly. Whereas animals from the control group presented growing tumors reaching over 800 mm³ on average on Day 42, mice treated with the MAGE-A1-specific TCR-T cells showed a robust anti-tumor response.

In a similar experiment, female NCG animals were implanted with Hs695T cells (HLA-A*02:01-positive cells expressing PRAME) and received a single dose of PRAME-specific TCR-T cells or of donor-matched, non-engineered control T cells (20E6 T cells, one day after randomization). Animals injected with TCR-T cells presented an anti-tumor response when compared to the control T cells-treated animals.

These studies confirmed that intravenous injection of TCR-T cells can successfully control the growth of pMHC-positive tumors inoculated subcutaneously in mice, confirming the potency of individual TCR-T cells.

In vitro experiments were conducted to demonstrate the value of combining TCR-T cells to treat a heterogenous tumor. Reporter HEK293T cells expressing exclusively HLA-A*02:01 and expressing a granzyme B-activated infrared fluorescent protein (IFP) were further engineered to express either MAGE-A1 or PRAME. The MAGE-A1 expressing cells were GFP-labeled, and the PRAME-positive cells were labeled with both GFP and CellTrace™ Violet to enable tracking in downstream flow cytometry readouts. When a TCR-T cell recognizes a target cell, it secretes cytotoxic granules into the target cell, triggering the target cell to become fluorescent (IFP-positive). The two target cells (i.e., PRAME- or MAGE-A1 positive) were mixed at a balanced ratio and co-cultured with MAGE-A1 TCR-T cells, PRAME TCR-T cells, or a multiplex product combining the two TCR-T cells. PRAME TCR-T cells and MAGE-A1 TCR-T cells were engineered from T cells from the same donor. The TCR-T cells corresponded to pan T cells engineered by lentivirus transduction using a delivery vector employing MGTM modification and co-delivering CD8α and CD8β co-receptor as described above (such as a Table 1). Donor-matched non-engineered T cells (NTC) were also included as a control. The experiment then measured the proportion of each subset of target (GFP-positive, MAGE-A1 target; GFP/CTV-positive, PRAME target) getting recognized and targeted by TCR-T cells as measured by the proportion of GFP or GFP/CTV becoming IFP-positive. NTC did not induce any IFP-positivity in either target subset. When MAGE-A1 TCR-T cells were co-cultured with the mixed target cells, the proportion of GFP-positive positive for IFP increased, but not that of GFP/CTV-positive cells. These results demonstrate that only the MAGE-A1-positive subset was recognized under this coculture condition. Conversely, when the PRAME TCR-T cells were co-cultured with the target cell mixture, the proportion of GFP/CTV-positive target also positive for IFP increased, but not that of GFP-positive target cells. These results confirm that only the PRAME-positive target cell subset was targeted under this coculture condition. Lastly, upon co-culture of a mixture of PRAME TCR-T cells and MAGE-A1 TCR-T cells with the mixed target cells, both the GFP-positive and GFP/CTV-positive cell subsets presented with IFP-positive signal, revealing that both target cell subsets were effectively recognized. The proportion of each subset becoming IFP-positive in the co-culture with the multiplexed product was comparable to what was observed when cocultured with each individual TCR-T cell product.

Altogether, the data demonstrate that although each individual TCR-T cell product was able to target cells positive with the relevant pMHC, the multiplex product was needed to broadly target the heterogeneous mixtures of cancer cells.

The mixture of HEK293T cells expressing either MAGE-A1 or PRAME along with HLA-A*02:01 was also inoculated subcutaneously in female NCG mice. Once the tumor reached 100 mm³ on average, the animals were randomized and received a single intravenous injection of 20E6 MAGE-A1 TCR-T cells, 20E6 PRAME TCR-T cells, or of a multiplex product consisting of 10E6 MAGE-A1 TCR-T cells, and 10E6 PRAME TCR-T cells, or a multiplex product consisting of 20E6 of MAGE-A1 TCR-T cells and 20E6 PRAME TCR-T cells. A group of animals received an intravenous injection with 20E6 donor-matched, non-engineered T cells. The same effector T cells as those described above were used in these experiments. Tumor volumes were then measured biweekly. Animals in the control group displayed rapidly growing tumors; tumor volumes reached over 1000 mm³ at the end of the study (on Day 24 post inoculation). On the other hand, animals dosed with each individual TCR-T cell subsets presented with tumors growing at a slower rate, only reaching ˜600-750 mm³ at the end of the study. Animals receiving the multiplex TCR-T cell products achieved a broader, more durable response when compared to animals receiving individual TCR-T cell product with average tumor volumes of ˜500 mm³ (10E6 of each TCR-T) or ˜300 mm³ (20E6 of each TCR-T). Along with the in vitro data presented in FIG. 6 and described above, these data confirm that each TCR-T cells only target a subset of the heterogenous tumor, achieving a partial response: a subset of cancer cells remains resistant to the single agent TCR-T cells and drives relapse. On the other hand, by broadly targeting the two cell subsets of the heterogenous tumor, the multiplex TCR-T product prevent the selection of resistant cells and achieved a stronger anti-tumor response. Moreover, FIG. 6 illustrates the concept of an ImmunoBank-based approach to therapeutic TCR therapy, wherein several therapeutic TCRs addressing multiple cancer associated proteins in conjunction with HLA restrictions are utilized to enable customized combinations of TCRs therapies based on tumor biology for every patient. For each patient, treatment decisions are made by determining (a) which cancer-associated proteins (rows of the ImmunoBank) are expressed in their tumor(s), using immunohistochemistry (IHC) or reverse transcription polymerase chain reaction (RT-PCR) and (b) which HLA genes (columns of the ImmunoBank) are intact in their tumor(s) (i.e., have not undergone loss of heterozygosity [LOH] at the HLA locus) measured by genomic sequencing. Once the patient's tumor target and HLA profile are determined, multiple TCRs (e.g., 2 TCRs, 3 TCRs, etc.) are selected from the ImmunoBank to prepare a customized multiplex TCR-T cell drug product.

### Example 5: Multiplexed TCR-T Cell Therapy Targeting the Same Target Using Different TCRs Recognizing Epitopes Presented on Distinct HLAs Enhances the Activity of Adoptive T Cell Therapy in Pre-Clinical Models

The present Example is based in part on the recognition that adoptive cell transfer with genetically engineered T cells holds great promise for treating solid tumors. Patients positive for particular HLA alleles of interest, such as HLA-A*02:01 and HLA-C*07:02, are amenable to treatment with TCRs recognizing epitopes of a given target presented by such HLAs, such TSC-204-A0201 and TSC-204-C0702, respectively (e.g., by concomitant or successive infusions of the TCRs). Patients in which the particular HLA alleles occur on separate chromosomes (separate haplotypes) are more likely to resist against HLA loss, as tumor cells that lose both class I HLA haplotypes are targeted by natural killer (NK) cells (O'Connor et al. (2006) Immunol. 117:1-10). Additional TCR-T components, which address different targets and a broader range of HLA types, may be used to further enhance the combination of TCRs and enable a broader range of patients to be treated with multiplexed TCR-T.

The present Example presents development of multiplexed TCR-T cell therapy in which a patient is treated with multiple TCR-T cell products, chosen from a collection of pre-vetted TCRs matched to the patient's tumor antigens and HLA type, as a solution to address antigen heterogeneity. As a representative, non-limiting example, two different TCRs were selected for multiplexed TCR-T treatment, each of which targets a different epitope of the same target but presented by a different HLA allele. TSC-204-A0201 and TSC-204-C0702 were used in a form consisting of pan T cells (comprising both CD4⁺ and CD8⁺ T cells, engineered by transposon/transposase-mediated gene delivery, to express (1) the respective recombinant TCR, (2) recombinant CD8α and CD8β co-receptors to maximize the efficacy of the therapeutic product, (3) a CD34-derived epitope tag fused on the N-terminus of CD8α to facilitate tracking of engineered cells in vitro and in vivo, (4) a dominant negative type II TGFβ receptor (DN-TGFβRII) to address tumor microenvironment-mediated immune suppression, and (5) a mutated form of dihydrofolate reductase (DHFRdm) protein to facilitate enrichment of engineered cells during the manufacturing process. Nevertheless, the results shown in the data provided herein are believed to be attributable to the function of the TCRs themselves.

In vitro characterization of the TSC-204-A0201 and TSC-204-C0702 materials demonstrated that the TCR-T cells engage in a target-dependent response leading to the secretion of inflammatory cytokines, the expansion of the effector T cells, and ultimately, the killing of target cells (FIG. 7). Since the MAGE-A1-derived epitopes targeted by TSC-204-A0201 or TSC-204-C0702 are presented on the class I MHCs HLA-A*02:01 and HLA-C*07:02, respectively, the recombinant TCRs use the CD8αß co-receptors to engage the pMHCs. Helper (CD4+) T cells do not naturally express the CD8αß co-receptor. Exogenous CD8α and CD8β co-receptors are co-delivered to the engineered T cells along with the therapeutic TCR to facilitate the ability of CD4+ helper T cells to recognize the class I-restricted epitope. Engineered CD4+ T cells contained in TSC-204-A0201 and in TSC-204-C0702 undergo proliferation alongside engineered CD8+ cytotoxic T cells, demonstrating functional engagement of helper T cells. Further, because the engineered T cells express the DN-TGFβRII, TSC-204-A0201 and TSC-204-C0702 TCR-T cells are active even in the presence of TGFβ, an immuno-suppressive cytokine that may be observed in the microenvironment of solid tumors.

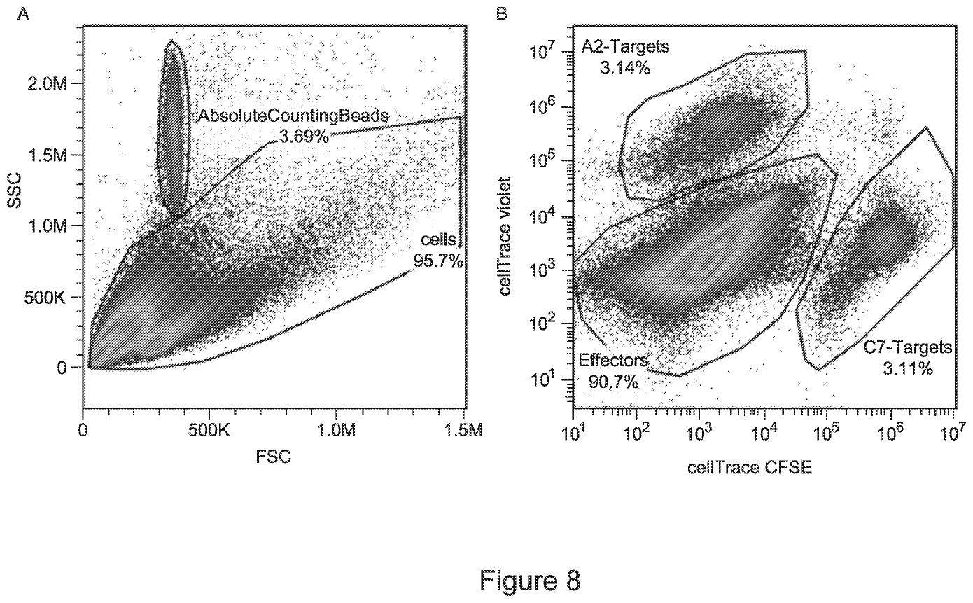

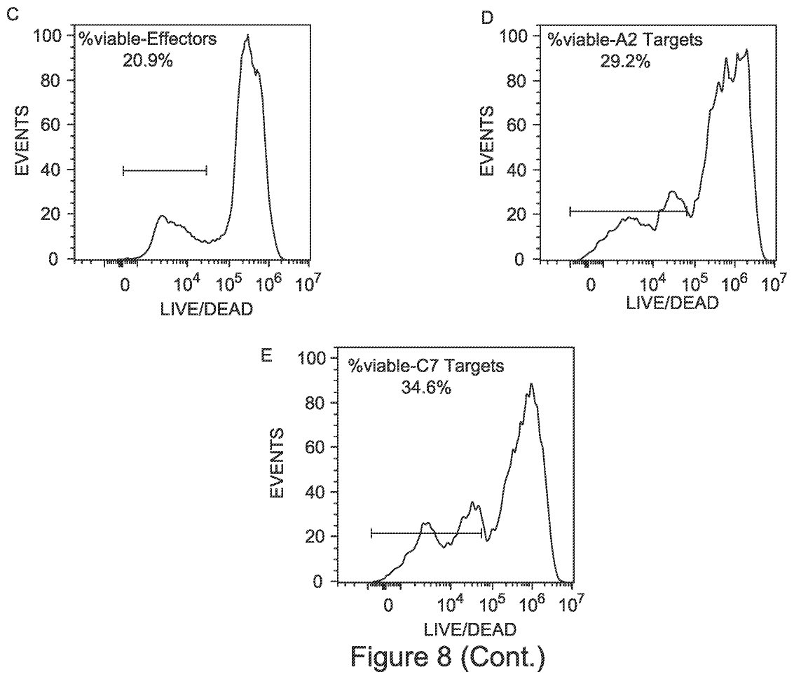

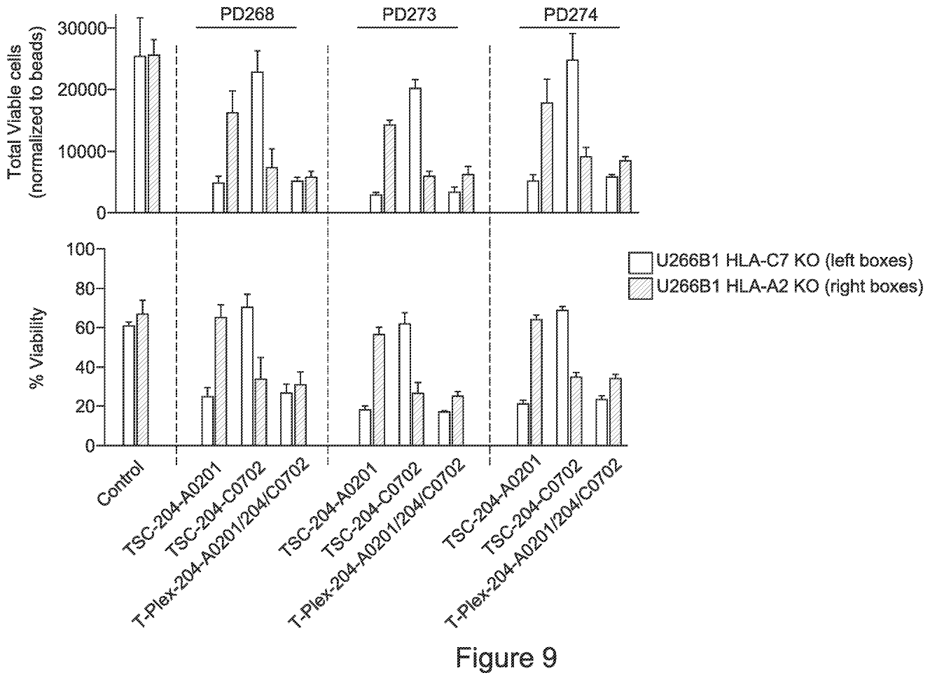

FIGS. 8 and 9 show results of TCR-T cells from three independent batches of TSC-204-A0201 and TSC-204-C0702 applied to a heterogenous target cancer cell population generated to simulate a MAGE-A1-positive tumor where LOH has occurred. Briefly, the MAGE-A1-positive, HLA-A*02:01-positive and HLA-C*07:02-positive cancer cell line U266B1 (i.e., cell line TIB-196 available from the ATCC) cell line was used. The cells were engineered by CRISPR knockout to create two versions of the cell line where only one of the two HLA of interest is intact (knocking out HLA-A*02:01 or HLA-C*07:02). U266B1 HLA-C*07:02 KO, “A2 Target”, and U266B1 HLA-A*02:01 KO, “C7 Target”, target cells were barcoded with CellTrace™ Violet and CFSE, respectively. After co-culture with singleplex (TSC-204-C0702 or TSC-204-A0201) or multiplex (T-Plex-204-A0201/204-C0702) conditions, cell suspensions were labeled with LIVE/DEAD™ viability dye to determine the viability of the target cells. Each target cell subset was labeled with distinct fluorescent dyes to be tracked in downstream flow cytometry readouts prior to being mixed at a 1:1 ratio.

Effector T cells were prepared. On the day prior to assay performance, effector TCR-T cells were thawed in a 37° C. water bath and washed with cytokine-free T cell medium. The cell concentration and viability (CCV) were determined, and viable TCR-T cells were seeded at a concentration of 1E6 cells/mL in a G-REX® 6M well plate in complete T cell medium. TCR-T cells were recovered in a humidified incubator at 37° C. and 5% CO₂ for 16-24 hours prior to culturing. After thawing and overnight recovery, effector TCR-T cells were harvested, washed with cytokine-free T cell medium and resuspended at 2E6 viable cells/mL in cytokine-free T cell medium to prepare for singleplex and multiplex conditions. Each TCR-T cell suspension was aliquoted for plating the positive controls single plex conditions. The remaining TCR-T cell suspensions were combined at a 1:1 ratio for all three batches to create the Test sample “T-Plex” conditions (2E6 viable cells/mL).

Similarly, target cell were prepared. Target cells were thawed, expanded, and maintained in culture for no more than 20 passages, then discarded. On the day prior to the start of co-culture, target cells were harvested and the CCV was measured and recorded. The targets cells were then seeded at 4E5 viable cells/mL to synchronize cell cycle phase. On the day of co-culture, target cells were harvested and the CCV were determined. The harvested cells were washed, and the cell density was adjusted to 1E6 cells/mL in protein-free PBS. Target U266B1 HLA-C*07:02 KO cells were labeled with cell trace violet and target U266B1 HLA-A*02:01 KO cells were labeled with CellTrace™ CFSE, both at 1:2000 according to the manufacturer's instructions and ultimately resuspended at 5E5 viable cells/mL in RPMI-based medium. After CellTrace™ labeling, each target cell suspension (5E5 viable cells/mL) were aliquoted for plating the negative controls. Heterogeneous target cell preparations were made from the remaining target cell suspensions (5E5 viable cells/mL), which were combined at a 1:1 ratio to be co-cultured with the positive controls, singleplex, and T-Plex test samples.

In addition, co-cultures were prepared. The heterogenous target cells were then co-cultured with different TCR-T cell mixtures made exclusively of TSC-204-A0201, of TSC-204-C0702 (corresponding to monotherapies or “singleplex” TCR-T cell products), or of a balanced mixture of TSC-204-A0201 and TSC-204-C0702 (i.e., “multiplex” TCR-T cell product). Briefly, target cells were plated in a sample well (U-bottom 96-well plate) and then singleplex condition or multiplex condition effector cell suspensions were added on top of the target cells. The final volume was 20 0 uL/well consisting of a 50/50 mix of target cells (10 0 μL) and effector cells (100 μL) in target cell RPMI media and cytokine-free T cells and target cell media, respectively. Cells were returned to the incubator and the co-culture incubated for 20-24 hours. Each positive control or test sample wells contained the combined target suspension of 5E4 total viable cells consisting of 50% CTV-labeled, U266B1 HLA-C*07:02 KO (2.5E4 cells total) and 50% CTCFSE-labeled, U266B1 HLA-A*02:01 KO (2.5E4 cells total). Each singleplex condition sample wells contained 2E5 total viable cells of the effector cell suspension, TSC-204-A0201, or TSC-204-C0702, combined with 5E4 total viable cells of the combined target cell suspension. This represents a total effector to target (E: T) ratio of 4:1 and an effector to specific target ratio of 8:1. Each T-Plex condition sample wells contained 2E5 total cells of the combined cell suspension, T-Plex-204-A0201/204-C0702, consisting of 50% TSC-204-A0201 (1E5 cells total) and 50% TSC-204-C0702 (1E5 cells total). Additionally, this well was combined with 5E4 total cells of the combined target cell suspension. This represents an E: T Ratio of 4:1 and an effector to specific target ratio of 4:1.

The cytotoxic activity of the TCR-T cells against the target cells was evaluated by flow cytometry by assessing the relative composition of each target cell population in the residual cells. At the end of co-culture, cells were pelleted by centrifugation then resuspended in LIVE/DEAD™ viability dye for 20 minutes, protected from light, at 4° C. to determine the viability of the cells. After one wash the cells were resuspend in EasySep™ and CountBright™ Absolute counting beads were added according to the manufacturer's instructions. The assay plate was acquired on the cytometer immediately after the counting beads were added. Data acquisition was performed on a CytoFLEX S flow cytometer following the machine's SOP-PC-0001-Instrument SOP-Use and Maintenance of the CytoFLEX. Compensation was performed automatically with the CytExpert software. Flow cytometric analysis was performed with FlowJo v7.6.5, the statistics were exported to Microsoft Excel 2010 and analyzed. Selected analyzed data were graphed in GraphPad Prism (v5.02).

The gating strategy is illustrated in FIGS. 8A-8E. Briefly, cells were gated from the FSC versus SSC dot plot. Subpopulations were distinguished using the CellTrace™ CFSE versus CellTrace™ Violet plot; C0702 Targets (CellTrace™ CFSE⁺/CellTrace™ Violet), A0201 Targets (CellTrace™ CFSE-/CellTrace™ Violet⁺) and effectors (CellTrace™ CFSE-/CellTrace™ Violet). Viable cells for each subpopulation were identified using the LIVE/DEAD™ histogram. Dead cells have a high fluorescence intensity because the LIVE/DEAD™ dye reacts with the free intracellular and extracellular amines of the compromised cell membrane. Viable cells can be distinguished as they display a lower fluorescence intensity since the dye is restricted to only the extracellular amines.

The killing of target cells was defined by the percent killing determined by subtracting the percent viability of the test sample from the negative control viability, then dividing by the negative control. When the percent viability for the test sample increased above the negative control viability value, baseline, the percent killing value was reported as 0% killing.

To quantify the absolute count of viable target cells acquired from a sample well, 20 μL of CountBright™ Absolute counting beads (20,400 beads/20 μL) were added to a 120 μL volume of cell suspension. The volume of cell sample acquired was multiplied by the absolute cell count concentration to determine the total viable cell count acquired.

Baseline viability of target cells was determined using negative controls. Briefly, CellTrace™ CFSE-labeled U266B1 HLA-A*02:01 KO targets (i.e., “C7 Targets”), are intact for the HLA-C*0702 along with MAGE-A1 protein. These constitute target cells for TSC-204-C0702 TCR-T cells. CellTrace™ Violet-labeled U266B1 HLA-C*07:02 KO targets (i.e., “A2 Targets”), express HLA-A*02:01 and MAGE-A1 protein. These constitute target cells for TSC-204-A0201 TCR-T cells. To determine the baseline viability of each individual target after overnight culture, negative controls were created; TSC-204-C0702 were co-cultured with the U266B1 HLA-C*07:02 KO target cells (“A2 target”) only, and TSC-204-A0201 were co-cultured with the U266B1-A*02:01 KO target cells (“C7 target”) only. The average baseline viability for U266B1 HLA-A*02:01 KO (C7 target) was 67.57% (n=3) and 61.53% (n=3) for U266B1 HLA-C*07:02 KO (A2 Target). The total viable cell count and percent viability are shown as data for “controls” in FIG. 9. These baseline values were used to calculate the specific TCR-T cell-mediated killing.

Similarly, TCR-T cell-mediated killing of target cells were observed using positive controls. Briefly, to determine the benefit of T-Plex-204-A0201/204-C0702-mediated killing, a cell suspension consisting of 50% CTV-labeled, U266B1 HLA-C*07:02 KO and 50% CTCFSE-labeled, U266B1 HLA-A*02:01 KO was created. The resulting target was heterogenous as a subset of the cells expressed HLA-A*02:01 but not HLA-C*07:02, and the other subset expressed HLA-C*07:02 but have lost HLA-A*02:01. This target combination “A2+C7” was co-cultured with TCR-T cell products consisting of TSC-204-A0201 or TSC-204-C0702 as monotherapies (“singleplex”) to demonstrate the killing ability of each individual component of T-Plex. These conditions served as the positive controls.

The percent killing results (calculated as the decrease of viability relative to the baseline as described above) as related to the total viable cell count and percent viability are shown in FIG. 9. In particular, TSC-204-A0201 TCR-T cells from three independent batches demonstrated specific cell-mediated killing of U266B1 HLA-C*07:02 KO target cells (58.88%, 69.01% and 64.30%, respectively), but did not kill the U266B1 HLA-A*02:01 KO target cells (0% specific killing across all 3 batches tested). Similarly, TSC-204-C0702 TCR-T cells from three independent batches demonstrated cell mediated killing of U266B1 HLA-A*02:01 KO target cells (48.84%, 59.84% and 47.66%, respectively), but systematically spared the U266B1 HLA-C*07:02 KO (0% specific killing across all 3 batches tested). These data confirmed that, in isolation, each individual TCR-T cell component of T-Plex selectively kills the target cells intact for the relevant HLA.

To determine the tumor killing ability of T-Plex-204-A0201/204-C0702, donor-matched individual TCR-T cell components from three independent batches of process-representative material were combined and co-cultured with the heterogeneous target combination “A2+C7”. As shown in FIG. 9, all three batches displayed a similar trend confirming that the viability of the heterogeneous target cell mixture decreased with all three batches of T-Plex-204-A0201/204-C0702. Barcoding the two target cell populations enabled analysis specifically of the viability of the A2 and C7 target subsets. U266B1 HLA-C*07:02 KO target cells decreased in viability when co-cultured with T-Plex-204-A0201/C0702 or with the isolated TSC-204-A0201 TCR-T cells. Similarly, the viability of U266B1 HLA-A*02:01 KO target cells decreased when co-cultured with T-Plex-204-A0201/C0702 or with the isolated TSC-204-C0702 TCR-T cells.

When compared to the baseline viability mentioned above, the specific killing (calculated as the decrease of viability relative to the baseline described above demonstrated that the T-Plex-204-A0201/204-C0702 products from the three independent batches had a specific tumor killing activity with U266B1 HLA-C*07:02 KO target cells of 55.31%, 71.18%, and 60.89%, respectively, and with U266B1 HLA-A*02:01 KO target cells of 53.38%, 61.77% and 48.45%, respectively. Notably, the individual killing activity of TSC-204-C0702 and TSC-204-A0201 TCR-T cell products were comparable to the combined killing activity of T-Plex-204-A0201/204-C0702, indicating effective function of the combined two TCR-T cell components. As described above, the assays are relatively short-term in duration and it is believed that further synergistic activity (such as from cytokine-dependent phenomena described herein) may be observed in other assays, such as, for example, reducing the effector to target ratio of one or more TCRs of a combination of TCRs to show the supportive effects of the other TCR(s) on the reduced TCR(s), in longer duration studies, and the like.

Thus, the results provided in FIG. 9 demonstrate that, when facing a heterogenous target cell population, a single TCR-T cell component effectively tackles a fraction of the tumor cells, sparing the subset of cells that cannot be recognized by the TCR-T cells (here because the cells lost the relevant HLA). Multiplexed TCR-T therapy (here, combining TSC-204-A0201 and TSC-204-C0702) led to the killing of both cancer cell subsets simultaneously. Therefore, the data establish that multiple TCR-T therapies, such as TSC-204-A0201 and TSC-204-C0702, can be combined to treat tumor where LOH has taken place to maximize the chances of reaching a complete response.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. A composition comprising at least two binding proteins, wherein i) at least one of the binding proteins is a T cell receptor (TCR) that is capable of binding to an immunogenic peptide derived from a target protein as an immunogenic peptide-MHC (pMHC) complex and ii) at least one of the binding proteins is a TCR that is capable of binding to a different immunogenic peptide from the same target protein or a different target protein than in i) as a pMHC complex, optionally wherein the composition comprises 2, 3, 4, 5, 6, 7, 8, 9, 10, 11, 12, 13, 14, 15, 16, 17, 18, 19, 20 or more binding proteins.

2. The composition of claim 1, wherein the binding affinity of the TCR of i) and/or the TCR of ii) has a K_{d} less than or equal to about 5×10⁻⁴ M.

3. The composition of claim 1 or 2, wherein the MHC of i) and the MHC of ii) are the same or different.

4. The composition of any one of claims 1-3, wherein the binding protein of i) and/or ii) comprises:
a) a T cell receptor (TCR) alpha chain CDR sequence with at least about 80% identity to a TCR alpha chain CDR sequence selected from the group consisting of TCR alpha chain CDR sequences listed in Table 1, Table 4, or Table 6; and/or
b) a TCR beta chain CDR sequence with at least about 80% identity to a TCR beta chain CDR sequence selected from the group consisting of TCR beta chain CDR sequences listed in Table 1, Table 4, or Table 6.

5. The composition of any one of claims 1-4, wherein the binding protein of i) and/or ii) comprises:
a) a TCR alpha chain variable (V_{α}) domain sequence with at least about 80% identity to a TCR V_{α} domain sequence selected from the group consisting of TCR V_{α} domain sequences listed in Table 1, Table 4, or Table 6; and/or
b) a TCR beta chain variable (V_{β}) domain sequence with at least about 80% identity to a TCR VB domain sequence selected from the group consisting of TCR VB domain sequences listed in Table 1, Table 4, or Table 6.

6. The composition of any one of claims 1-5, wherein the binding protein of i) and/or ii) comprises:
a) a TCR alpha chain sequence selected from the group consisting of TCR alpha chain sequences listed in Table 1, Table 4, or Table 6; and/or
b) a TCR beta chain sequence selected from the group consisting of TCR beta chain sequences listed in Table 1, Table 4, or Table 6.

7. The composition of any one of claims 1-6, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 1, Table 2, Table 4, and/or Table 6) the TCR beta chain CDR, TCR VB domain, and/or TCR beta chain is encoded by a TRBV, TRBJ, and/or TRBC gene or fragment thereof selected from the group of TRBV, TRBJ, and TRBC genes listed in Table 1, Table 2, Table 4, and/or Table 6) each CDR of the binding protein has up to five amino acid substitutions, insertions, deletions, or a combination thereof as compared to the cognate reference CDR sequence listed in Table 1, Table 4, or Table 6.

8. The composition of any one of claims 1-7, wherein the immunogenic peptide comprises an amino acid sequence shown in Table 3.

9. The composition of any one of claims 1-8, wherein the binding protein is chimeric, humanized, or human.

10. The composition of any one of claims 1-9, wherein the binding protein is a TCR, an antigen-binding fragment of a TCR, a single chain TCR (scTCR), a chimeric antigen receptor (CAR), or a fusion protein comprising a TCR and an effector domain, optionally wherein the binding domain comprises a transmembrane domain and an effector domain that is intracellular.

11. The composition of any one of claims 1-10, wherein the TCR alpha chain and the TCR beta chain are covalently linked, optionally wherein the TCR alpha chain and the TCR beta chain are covalently linked through a linker peptide.

12. The composition of any one of claims 1-11, wherein the TCR alpha chain and/or the TCR beta chain are covalently linked to a moiety, optionally wherein the covalently linked moiety comprises an affinity tag or a label.

13. The composition of claim 12, wherein the affinity tag is selected from the group consisting of CD34 enrichment tag, Glutathione-S-Transferase (GST), calmodulin binding protein (CBP), protein C tag, Myc tag, HaloTag, HA tag, Flag tag, His tag, biotin tag, and V5 tag, and/or wherein the label is a fluorescent protein.

14. The composition of any one of claims 1-13, wherein the covalently linked moiety is selected from the group consisting of an inflammatory agent, cytokine, toxin, cytotoxic molecule, radioactive isotope, or antibody or antigen-binding fragment thereof.

15. The composition of any one of claims 1-14, wherein the binding protein binds to the pMHC complex on a cell surface.

16. The composition of any one of claims 1-15, wherein the MHC is a MHC multimer, optionally wherein the MHC multimer is a tetramer.

17. The composition of any one of claims 1-16, wherein the MHC is a MHC class I molecule.

18. The composition of any one of claims 1-17, wherein the MHC comprises an MHC alpha chain that is an HLA serotype HLA-A*02.

19. The composition of any one of claims 1-18, wherein the HLA allele is selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, HLA-B*0721, HLA-C*0702, HLA-C*0701, HLA-C*0401, HLA-C*0602, HLA-C*0304, HLA-C*0501, HLA-C*1601, HLA-C*0202, HLA-C*0303, HLA-C*1203, HLA-C*0802, HLA-C*0102, HLA-C*1701, HLA-C*1502, HLA-C*1402, HLA-C*1202, HLA-C*0704, HLA-C*0801, HLA-C*0302, HLA-C*1801, HLA-C*1505, HLA-C*1602, HLA-C*0804, HLA-C*0305, and HLA-C*1403 allele.

20. The composition of any one of claims 1-19, wherein binding of the composition to the pMHC complexes elicits an immune response that is greater than either TCR alone, optionally wherein the immune response is a T cell response and/or a synergistic response.

21. The composition of any one of claims 1-20, wherein the T cell response is selected from the group consisting of T cell expansion, cytokine release, and/or cytotoxic killing.

22. The composition of any one of claims 1-21, the binding protein is capable of specifically binding to the immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M.

23. The composition of any one of claims 1-22, wherein the binding protein has a higher binding affinity to the peptide-MHC (pMHC) than does a known T-cell receptor.

24. The composition of any one of claims 1-23, wherein the binding protein has at least 1.05-fold higher binding affinity to the peptide-MHC (pMHC) than does a known T-cell receptor.

25. The composition of any one of claims 1-24, wherein the binding protein induces higher T cell expansion, cytokine release, and/or cytotoxic killing than does a known T-cell receptor when contacted with target cells with a heterozygous expression of the target.

26. The composition of any one of claims 1-25, wherein the binding protein induces at least 1.05-fold increase in T cell expansion, cytokine release, and/or cytotoxic killing than does a known T-cell receptor when contacted with target cells with a heterozygous expression of the target.

27. The composition of claim 25 or 26, wherein the target cell is a SK-MEL-5, DEL, THP-1, or TF-1 cell line.

28. The composition of claim 25 or 26, wherein the target cell is a cancer cell.

29. The composition of claim 28, wherein the cancer is a hematological malignancy.

30. The composition of claim 28, wherein the cancer is a solid tumor.

31. The composition of claim 30, wherein the cancer is a melanoma, head and neck cancer, or lung caner.

32. An isolated nucleic acid molecule that hybridizes, under stringent conditions, with the complement of a nucleic acid encoding a polypeptide selected from the group consisting of polypeptide sequences listed in Table 1, Table 4, or Table 6, or a polypeptide composition of any one of claims 1-31, or a sequence with at least about 80% homology to a nucleic acid encoding a polypeptide selected from the group consisting of the polypeptide sequences listed in Table 1, Table 4, or Table 6, optionally wherein the isolated nucleic acid molecule comprises 1) a TRAV, TRAJ, and/or TRAC gene or fragment thereof selected from the group of TRAV, TRAJ, and TRAC genes listed in Table 1, Table 2, Table 4, and/or Table 6 and/or 2) a TRBV, TRBJ, and/or TRBC gene or fragment thereof selected from the group of TRBV, TRBJ, and TRBC genes listed in Table 1, Table 2, Table 4, and/or Table 6, or a polypeptide composition of any one of claims 1-31.

33. The isolated nucleic acid of claim 32, wherein the nucleic acid is codon optimized for expression in a host cell.

34. A vector comprising the isolated nucleic acid of claim 32 or 33.

35. The vector of claim 34, wherein the vector is a cloning vector, expression vector, or viral vector.

36. The vector of claim 34 or 35, wherein the vector further comprises nucleic acid sequence encoding CD8α, CD8β, a dominant negative TGFβ receptor II (DN-TGFβRII), selectable protein marker, optionally wherein the selectable protein marker is dihydrofolate reductase (DHFR).

37. The vector of any one of claims 34-36, wherein the nucleic acid sequence encoding CD8α, CD8β, the DN-TGFβRII, and/or the selectable protein marker is operably linked to a nucleic acid encoding a tag.

38. The vector of any one of claims 34-37, wherein the nucleic acid encoding a tag is at the 5′ upstream of the nucleic acid sequence encoding CD8α, CD8β, the DN-TGFβRII, and/or the selectable protein marker such that the tag is fused to the N-terminus of CD8α, CD8β, the DN-TGFβRII, and/or the selectable protein marker.

39. The vector of any one of claims 34-38, wherein the tag is a CD34 enrichment tag.

40. The vector of any one of claims 34-39, wherein the isolated nucleic acid of any preceding claim, and the nucleic acid sequence encoding CD8α, CD8β, the DN-TGFβRII, and/or the selectable protein marker are interconnected with an internal ribosome entry site or a nucleic acid sequence encoding a self-cleaving peptide.

41. The vector of any one of claims 34-40, wherein the self-cleaving peptide is P2A, E2A, F2A or T2A.

42. A host cell which comprises the isolated nucleic acid of claim 32 or 33, comprises the vector of any one of claims 34-41, and/or expresses the binding protein composition of any one of claims 1-31, optionally wherein the cell is genetically engineered, further optionally wherein the host cells is a collection of host cells each comprising an isolated nucleic acid of claim 32 or 33, comprises the vector of any one of claims 34-41, and/or expresses the binding protein composition of any one of claims 1-31.

43. The host cell of claim 42, wherein the host cell comprises a chromosomal gene knockout of a TCR gene, an HLA gene, or both.

44. The host cell of claim 42 or 43, wherein the host cell comprises a knockout of an HLA gene selected from an α1 macroglobulin gene, α2 macroglobulin gene, α3 macroglobulin gene, β1 microglobulin gene, β2 microglobulin gene, and combinations thereof.

45. The host cell of any one of claims 42-44, wherein the host cell comprises a knockout of a TCR gene selected from a TCR α variable region gene, TCR β variable region gene, TCR constant region gene, and combinations thereof.

46. The host cell of any one claims 42-45, wherein the host cell expresses CD8α, CD8β, a DN-TGFβRII, and/or a selectable protein marker, optionally wherein the selectable protein marker is DHFR.

47. The host cell of claim 46, wherein the CD8α, CD8β, the DN-TGFβRII, and/or the selectable protein marker is fused to a CD34 enrichment tag.

48. The host cell of claim 47, wherein host cells are enriched using the CD34 enrichment tag.

49. The host cell of any one of claims 42-48, wherein the host cell is a hematopoietic progenitor cell, peripheral blood mononuclear cell (PBMC), cord blood cell, or immune cell.

50. The host cell of any one of claims 42-49, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof.

51. The host cell of any one of claims 42-50, wherein the T cell is a naive T cell, central memory T cell, effector memory T cell, or a combination thereof.

52. The host cell of any one of claims 42-51, wherein the T cell is a primary T cell or a cell of a T cell line.

53. The host cell of any one of claims 42-52, wherein the T cell does not express or has a lower surface expression of an endogenous TCR.

54. The host cell of any one of claims 42-53, wherein the host cell is capable of producing a cytokine or a cytotoxic molecule when contacted with a target cell that comprises a peptide-MHC (pMHC) complex comprising the HA-2 peptide epitope in the context of an MHC molecule.

55. The host cell of claim 54, wherein the host cell is contacted with the target cell in vitro, ex vivo, or in vivo.

56. The host cell of claim 54 or 55, wherein the cytokine is TNF-α, IL-2, and/or IFN-γ.

57. The host cell of any one of claims 54-56, wherein the cytotoxic molecule is perforins and/or granzymes, optionally wherein the cytotoxic molecule is granzyme B.

58. The host cell of any one of claims 54-57, wherein the host cell is capable of producing a higher level of cytokine or a cytotoxic molecule when contacted with a target cell with a heterozygous expression of the target.

59. The host cell of claim 58, wherein the host cell is capable of producing an at least 1.05-fold higher level of cytokine or a cytotoxic molecule.

60. The host cell of any one of claims 54-59, wherein the host cell is capable of killing a target cell that comprises a peptide-MHC (pMHC) complex comprising the target peptide epitope in the context of an MHC molecule.

61. The host cell of claim 60, wherein the killing is determined by a killing assay.

62. The host cell of claim 60 or 61, wherein the ratio of the host cell and the target cell in the killing assay is from 20:1 to 0.625:1.

63. The host cell of any one of claims 60-62, wherein a target cell is a T2 cell pulsed with 1 μg/mL to 50 pg/mL of immunogenic peptide.

64. The host cell of any one of claims 60-62, wherein the host cell is capable of killing a higher number of target cells when contacted with target cells with a heterozygous expression of the target.

65. The host cell of claim 64, wherein the host cell is capable of killing an at least 1.05-fold higher number of target cells.

66. The host cell of any one of claims 60, 61, 64 and 65, wherein the target cell is a SK-MEL-5, DEL, THP-1, or TF-1 cell line.

67. The host cell of any one of claims 54-66, wherein the immunogenic peptide comprises an amino acid sequence shown in Table 3.

68. The host cell of any one of claims 54-67, wherein the MHC molecule is a MHC class I molecule.

69. The host cell of any one of claims 54-68, wherein the MHC molecule comprises an MHC alpha chain that is an HLA serotype HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, or HLA-C*18.

70. The host cell of any one of claims 54-69, wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, HLA-B*0721, HLA-C*0702, HLA-C*0701, HLA-C*0401, HLA-C*0602, HLA-C*0304, HLA-C*0501, HLA-C*1601, HLA-C*0202, HLA-C*0303, HLA-C*1203, HLA-C*0802, HLA-C*0102, HLA-C*1701, HLA-C*1502, HLA-C*1402, HLA-C*1202, HLA-C*0704, HLA-C*0801, HLA-C*0302, HLA-C*1801, HLA-C*1505, HLA-C*1602, HLA-C*0804, HLA-C*0305, and HLA-C*1403 allele.

71. The host cell of any one of claims 54-70, wherein the target cell is a cell line selected from the group consisting of SK-MEL-5, Del, THP-1, and TF-1 cell lines, or is a cancer cell expressing the immunogenic peptide.

72. The host cell of claim 71, wherein the cancer cell is of a hematological malignancy or a solid tumor.

73. The host cell of any one of claims 54-71, wherein the host cell does not express the immunogenic peptide, is not recognized by a binding protein of any one of claims 1-30, is not of serotype list in claim 69, and/or does not express an HLA allele listed in claim 70.

74. A population of host cells of any one of claims 42-73.

75. A method of preventing and/or treating a non-malignant disorder, a hyperproliferative disorder or a relapse of a hyperproliferative disorder characterized by expression of an immunogenic peptide antigen in a subject comprising administering to the subject a therapeutically effective amount. of a combination of a composition of any one of claims 1-74, or a combination of binding proteins, nucleic acids, vectors, and/or host cells according to any one of claims 1-74.

76. The method of claim 75, wherein the composition comprises cells, optionally wherein the cell is an allogeneic cell, syngeneic cell, or autologous cell.

77. The method of claim 75 or 76, wherein the cell is genetically modified.

78. The method of any one of claims 75-77, wherein the cell comprises a chromosomal gene knockout of a TCR gene, an HLA gene, or both a TCR gene and an HLA gene.

79. The method of any one of claims 75-78, wherein the cell comprises a knockout of an HLA gene selected from an α1 macroglobulin gene, α2 macroglobulin gene, α3 macroglobulin gene, β1 microglobulin gene, β2 microglobulin gene, and a combination thereof.

80. The method of any one of claims 75-79, wherein the cell comprises a knockout of a TCR gene selected from a TCR α variable region gene, TCR β variable region gene, TCR constant region gene, and combinations thereof.

81. The method of any one claims 75-80, wherein the cell expresses CD8α, CD8β, a DN-TGFβRII, and/or a selectable protein marker, optionally wherein the selectable protein marker is DHFR and further optionally wherein the CD8α, CD8β, the DN-TGFβRII, and/or the selectable protein marker is fused to a CD34 enrichment tag.

82. The method of claim 81, wherein cells are enriched using the CD34 enrichment tag.

83. The method of any one of claims 75-82, wherein the cell is a hematopoeitic progenitor cell, peripheral blood mononuclear cell (PBMC), cord blood cell, or immune cell.

84. The method of any one of claims 75-83, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof.

85. The method of any one of claims 75-84, wherein the T cell is a naive T cell, central memory T cell, effector memory T cell, or combination thereof.

86. The method of any one of claims 75-85, wherein the T cell is a primary T cell or a cell of a T cell line.

87. The method of any one of claims 75-86, wherein the T cell does not express or has a lower surface expression of an endogenous TCR.

88. The method of any one of claims 75-87, wherein the cell is capable of producing a cytokine or a cytotoxic molecule when contacted with a target cell that comprises a peptide-MHC (pMHC) complex comprising the immunogenic peptide in the context of an MHC molecule.

89. The method of any one of claims 75-89, wherein the cytokine is TNF-α, IL-2, and/or IFN-γ.

90. The method of any one of claims 75-90, wherein the cytotoxic molecule is perforins and/or granzymes, optionally wherein the cytotoxic molecule is granzyme B.

91. The method of any one of claims 75-90, wherein the cell is capable of producing a higher level of cytokine or a cytotoxic molecule when contacted with a target cell with a heterozygous expression of the target.

92. The method of claim 91, wherein the cell is capable of producing an at least 1.05-fold higher level of cytokine or a cytotoxic molecule.

93. The method of any one of claims 75-92, wherein the host cell is capable of killing a target cell that comprises a peptide-MHC (pMHC) complex comprising the target in the context of an MHC molecule.

94. The method of any one of claims 75-93, wherein the host cell is capable of killing a higher number of target cells when contacted with target cells with a heterozygous expression of the target.

95. The method of claim 94, wherein the host cell is capable of killing an at least 1.05-fold higher number of target cells.

96. The method of any one of claims 75-95, wherein the immunogenic peptide comprises an amino acid sequence shown in Table 3.

97. The method of any one of claims 92-96, wherein the MHC molecule is an MHC class I molecule.

98. The method of any one of claims 75-97, wherein the MHC molecule comprises an MHC alpha chain that is an HLA serotype HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, or HLA-C*18.

99. The method of any one of claims 75-98, wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, HLA-B*0721, HLA-C*0702, HLA-C*0701, HLA-C*0401, HLA-C*0602, HLA-C*0304, HLA-C*0501, HLA-C*1601, HLA-C*0202, HLA-C*0303, HLA-C*1203, HLA-C*0802, HLA-C*0102, HLA-C*1701, HLA-C*1502, HLA-C*1402, HLA-C*1202, HLA-C*0704, HLA-C*0801, HLA-C*0302, HLA-C*1801, HLA-C*1505, HLA-C*1602, HLA-C*0804, HLA-C*0305, and HLA-C*1403 allele.

100. The method of any one of claims 75-99, wherein the target cell is a non-malignant cell or a hyperproliferating cell expressing the antigen in the subject.

101. The method of any one of claims 75-100, wherein the composition further comprises a pharmaceutically acceptable carrier.

102. The method of any one of claims 75-101, wherein the composition induces an immune response against the non-malignant cells or the hyperproliferating cells expressing the antigen in the subject that is greater than either TCR alone, optionally wherein the immune response is a synergistic response.

103. The method of any one of claims 75-102, wherein the composition induces an antigen-specific T cell immune response against the non-malignant cells or the hyperproliferating cells expressing the antigen in the subject.

104. The method of any one of claims 75-103, wherein the antigen-specific T cell immune response comprises at least one of a CD4⁺ helper T lymphocyte (Th) response and a CD8⁺ cytotoxic T lymphocyte (CTL) response.

105. The method of any one of claims 75-104, wherein the hyperproliferative disorder comprises a hematological malignancy or a solid tumor.

106. The method of any one of claims 75-104, wherein the non-malignant disorder is an autoimmune disorder, optionally wherein the autoimmune disorder is systemic sclerosis or multiple sclerosis.

107. The method of any one of claims 75-106, wherein the subject is receiving or previously received a hematopoietic cell transplant (HCT), optionally wherein the HCT comprises cells that do not express the immunogenic antigen, are not recognized by a binding protein of any one of claims 1-31, are not of serotype listed in claim 98, and/or do not express an HLA allele listed in claim 99.

108. The method of claim 107, wherein the HCT comprises a donor hematopoeitic cell comprising a chromosomal knockout of a gene that encodes an HLA component, a chromosomal knockout of a gene that encodes a TCR component, or both.

109. The method of any one of claims 75-108, further comprising administering at least one additional treatment for the non-malignant disorder, the hyperproliferative disorder or the relapse of a hyperproliferative disorder to the subject.

110. The method of any one of claims 75-109, wherein the at least one additional treatment for the non-malignant disorder, the hyperproliferative disorder or the relapse of a hyperproliferative disorder is administered concurrently or sequentially with the composition.

111. The method of any one of claims 75-110, wherein the subject is an animal model of disorder characterized by the immunogenic antigen expression and/or a mammal, optionally wherein the mammal is a human, a primate, or a rodent.
